(19) United States
(12) Patent Application Publication
(10) Pub. No.: US 2005/0256000 A1
(43) Pub. Date: Nov. 17, 2005

(54) Quinoxalin-2-one derivatives, compositions which protect useful plants and comprise these derivatives, and processes for their preparation and their use

(75) Inventors: Wolfgang Schaper, Diedorf (DE); Lothar Willms, Hofheim (DE); Christopher Rosinger, Hofheim (DE); Erwin Hacker, Hochheim (DE); Eckhard Rose, Liederbach (DE); Dirk Schmutzler, Hattersheim (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/127,016

(22) Filed: May 11, 2005

(30) Foreign Application Priority Data
May 12, 2004 (DE) 10 2004 023 332.2

(51) Int. Cl. A01N 25/32; C07D 241/36

(52) U.S. Cl. 504/105; 544/334

(57) ABSTRACT

Compounds of the formula (I) and salts thereof 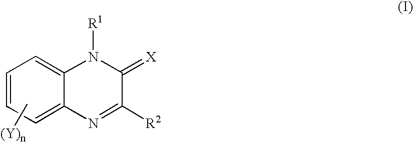 in which
X is O or S; (Y)ₙ=n substituents Y, n is 0, 1, 2, 3 or 4,
R¹ is H, OH, NH₂, (C₁-C₄)-alkylamino, di-[(C₁-C₄)-alkyl]amino or optionally substituted (C₁-C₁₀)-alkyl, (C₃-C₁₀)-alkenyl, (C₃-C₁₀)-alkynyl or (C₁-C₁₀)-alkoxy, (C₃-C₁₀)-cycloalkyl, (C₄-C₁₀)-cycloalkenyl, aryl or heterocyclyl,
R² is H or optionally substituted (C₁-C₁₀)-alkyl, (C₃-C₁₀)-alkenyl, (C₃-C₁₀)-alkynyl, (C₃-C₁₀)-cycloalkyl, (C₄-C₁₀)-cycloalkenyl, aryl or heterocyclyl, where the radicals Y are as defined in claim 1 are suitable for use as safeners for crop plants or useful plants against the phytotoxic actions of agrochemicals such as pesticides in these plants.

The present invention relates to compositions which protect useful plants and comprise quinoxalinone derivatives, specifically 1,2-dihydroquinoxalin-2-one derivatives, as safeners and, if appropriate, pesticides, and also to certain quinoxalinone derivatives and to processes for their preparation.

When controlling unwanted organisms in crops of plants which are useful for agriculture or forestry by using pesticides, the useful plants are frequently also damaged to a greater or lesser extent, in a manner which is unwanted per se, by the pesticides employed. This effect is encountered in particular with the use of a considerable number of herbicides in crops of useful plants such as, for example, corn, rice or cereals—and there primarily in the post-emergence application. In some instances, the useful plants can be protected against the phytotoxic properties of the pesticides by employing safeners or antidotes, without diminishing the pesticidal activity against the harmful organisms. In some cases, even an improved pesticidal action against harmful organisms such as weeds was observed.

The compounds which have hitherto been disclosed as safeners have various chemical structures. Thus, U.S. Pat. No. 4,902,340 discloses derivatives of quinolin-8-oxyalkanecarboxylic acids as safeners for herbicides from the group of the diphenyl ethers and the pyridyloxyphenoxypropionic acids and EP-A 0 520 371 discloses isoxazolines and isothiazolines as safeners for various types of herbicides, where the last-mentioned publication gives aryloxyphenoxycarboxylic acids, sulfonylureas and imidazolinones as preferred herbicides. Substituted benzo-fused five-membered and six-membered heterocycles as safeners are known from WO-A-98/13361. WO-A-99/00020 describes 3-(5-tetrazolylcarbonyl)-2-quinolinones and their use as safeners. DE 19621522.6 (WO-A-97/45016) and DE 19742951.3 (WO-A-99/16744) describe N-acylsulfonamides as safeners, preferably for protecting corn plants.

Active compounds from the chemical class of the quinoxalin-2-ones having pesticidal properties are known from the literature. Various biological actions are described; thus, for example, Pestic. Sci. 14 (1983), 135 mentions the fungicidal action of 1,6-dimethyl-3-phenyl-1,2-dihydroquinoxalin-2-one; U.S. Pat. No. 3,582,315 and U.S. Pat. No. 3,647,793 describe the herbicidal action of 1-alkyl-3-phenyl-1,2-dihydroquinoxalin-2-ones; GB 1574429 mentions the herbicidal action of 3-(2-thienyl)-1,2-dihydroquinoxalin-2-one.

Also known are representatives having pharmacological properties. Helv. Chim. Acta XXXV (1952) 2301, II Farmaco, Ed. Sci 40 (1985) 303, WO 99/50254, AT 226709 and AT 228204 describe the pharmacological actions of 1-dialkylaminoalkyl-3-phenyl- and -3-benzyldihydroquinoxalinones and of 1-hydroxyethyl-3-phenyl-1,2-dihydroquinoxalinone. WO 97/07116 describes the use of 1-aminoalkyl-3-aryl-1,2-dihydroquinoxalinones as inhibitors of prolylendopeptidase. WO 2002/002550 relates to the use of aryl-fused pyrazinones as kinase inhibitors. 1-Carboethoxy-methyl- and 1-carboxymethyl-3-aminophenyl-1,2-dihydroquinoxalinone derivatives are said to have antiamebic and diuretic action (Indian J. of Chem. (1974) 124). A use of such compounds as safeners has hitherto not been disclosed.

When safeners were used to protect useful plants against damage by pesticides, it was found that the known safeners may in many cases have disadvantages. These include:
the safener reduces the efficacy of the pesticides, in particular that of herbicides, against the harmful plants,
the useful-plant-protecting properties are insufficient,
in combination with a certain herbicide, the spectrum of the useful plants in which the safener/herbicide is to be employed is not sufficiently wide,
a certain safener can only be combined with a small number of herbicides,
by using safeners, the application rate to be applied and the amount of formulation is increased, which may cause problems during the application.

For the reasons mentioned, there is a need to provide alternative compositions which protect useful plants and comprise compounds having safener action and, if appropriate, pesticides.

The invention provides the use of compounds of the formula (I) or salts thereof (1,2-dihydroquinoxalin-2-one derivatives) 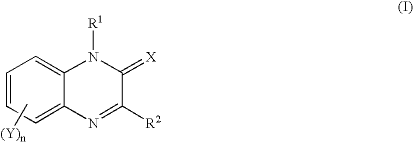
in which
X is oxygen or sulfur;
(Y)ₙ
   are n substituents Y,
   where each Y independently of the others is a halogen, cyano, nitro, (C₁-C₆)-alkyl, (C₂-C₆)-alkenyl, (C₂-C₆)-alkynyl, (C₁-C₆)-alkoxy, (C₁-C₆)-alkylthio, (C₁-C₆)-alkylsulfinyl, (C₁-C₆)-alkylsulfonyl, (C₁-C₆)-alkoxycarbonyl, (C₁-C₄)-alkylamino or di-[(C₁-C₄)-alkyl]amino radical,
      where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
   (C₃-C₆)-cycloalkyl, (C₄-C₆)-cycloalkenyl, aryl or heterocyclyl,
      where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkoxy-(C₁-C₄)-alkoxy and (C₁-C₄)-alkylthio, or
   two adjacent groups Y together with the carbon atoms which are directly attached are a four- to eight-membered fused-on ring which is carbocyclic or heterocyclic, has one or more, preferably one to three, hetero ring atoms from the group consisting of N, O and S and is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio,
n is 0, 1, 2, 3 or 4, preferably 0, 1, 2 or 3, and in particular 0, 1 or 2, and
R¹
   is hydrogen, hydroxyl, amino, (C₁-C₄)-alkylamino, di-[(C₁-C₄)-alkyl]amino, (C₁-C₁₀)-alkyl, (C₃-C₁₀)-alkenyl, (C₃-C₁₀)-alkynyl or (C₁-C₁₀)-alkoxy,
      where each of the 4 last-mentioned radicals is unsubstituted or substituted by one or more identical or different radicals R^{a} and, including substituents, has 1 to 30 carbon atoms, preferably 1 to 24 carbon atoms, or
   (C₃-C₁₀)-cycloalkyl, (C₄-C₁₀)-cycloalkenyl, aryl or heterocyclyl,
      where each of the 4 last-mentioned radicals is unsubstituted or substituted by one or more identical or different radicals R^{b} and, including substituents, has 3 to 30 carbon atoms, preferably 3 to 24 carbon atoms, and
R²
   is hydrogen, (C₁-C₁₀)-alkyl, (C₃-C₁₀)-alkenyl or (C₃-C₁₀)-alkynyl,
      where each of the 3 last-mentioned radicals is unsubstituted or substituted by one or more identical or different radicals R^{c} and, including substituents, has 1 to 30 carbon atoms, preferably 1 to 24 carbon atoms, or
   (C₃-C₁₀)-cycloalkyl, (C₄-C₁₀)-cycloalkenyl, aryl or heterocyclyl,
      where each of the 4 last-mentioned radicals is unsubstituted or substituted by one or more identical or different radicals R^{d} and, including substituents, has 3 to 30 carbon atoms, preferably 3 to 24 carbon atoms,
   where in the radicals R¹ and R²
   R^{a} in each case independently of other radicals R^{a} is an inorganic or organic radical, preferably a radical from the group consisting of halogen, cyano, nitro and radicals of the formulae -Z^{a}-R^{a}* and R^{cyc-a},
   R^{b} in each case independently of other radicals R^{b} is an inorganic or organic radical, preferably a radical from the group consisting of halogen, cyano, nitro and radicals of the formulae -Z^{b}-R^{b}* and R^{b}**,
   R^{c} in each case independently of other radicals R^{c} is an inorganic or organic radical, preferably a radical from the group consisting of halogen, cyano, nitro and radicals of the formulae -Z^{c}-R^{c}* and R^{cyc-c},
   R^{d} in each case independently of other radicals R^{d} is an inorganic or organic radical, preferably a radical from the group consisting of halogen, cyano, nitro and radicals of the formulae -Z^{d}-R^{d}* and R^{d}**
   where in the radicals R^{a}, R^{b}, R^{c} and R^{d}
   Z^{a}, Z^{b}, Z^{c} and Z^{d} are each independently of one another divalent functional monoatomic or polyatomic groups having at least one heteroatom and
   R^{cyc-a} and R^{cyc-c} are each an optionally substituted cyclic hydrocarbon radical having a total of 1 to 24 carbon atoms or an optionally substituted heterocyclic radical having a total of 1 to 24 carbon atoms and
   R^{a}*, R^{b}*, R^{c}*, R^{d}*, R^{b}** and R^{c}** are each independently of one another an optionally substituted hydrocarbon radical having a total of 1 to 24 carbon atoms or an optionally substituted heterocyclic radical having a total of 1 to 24 carbon atoms or
   R^{a}*, R^{b}*, R^{c}*, R^{d}* are each independently of one another hydrogen,
      as safeners, i.e. as agents for preventing or reducing phytotoxic actions of agrochemicals, preferably pesticides, in particular herbicides, in useful plants or crop plants.

If, by a hydrogen shift, the compounds are capable of forming tautomers whose structure is not formally covered by formula (I), these tautomers are nevertheless embraced by the definition of the compounds of the formula (I) according to the invention.

Depending on the nature and the attachment of the substituents, the compounds of the formula (I) may be present as stereoisomers. All possible stereoisomers defined by their specific spatial form, such as enantiomers, diastereomers, Z- and E-isomers, are embraced by the formula (I).

If, for example, one or more alkenyl groups are present, it is possible for diastereomers (Z- and E-isomers) to occur. If, for example, one or more asymmetric carbon atoms are present, it is possible for enantiomers and diastereomers to occur. Stereoisomers can be obtained by customary separation methods, for example by chromatographic separation procedures, from the mixtures obtained in the preparation. It is also possible to selectively prepare stereoisomers by employing stereoselective reactions using optically active starting materials and/or auxiliaries. Thus, the invention also relates to all stereoisomers embraced by the formula (I) but not shown in their specific stereoform, and mixtures thereof.

The possibilities of combining the various substituents of the formula (I) are to be understood in such a way that the general principles of the synthesis of chemical compounds are to be observed, i.e. the formula (I) does not embrace compounds of which the skilled worker knows that they are chemically impossible.

The compounds of the formula (I) are capable of forming salts. Salt formation may occur by action of the base on those compounds of the formula (I) which carry an acidic hydrogen atom, for example in the case where R¹ contains a COOH group or a sulfonamide group —NHSO₂—. Suitable bases are, for example, organic amines and also ammonium, alkali metal or alkaline earth metal hydroxides, carbonates and bicarbonates, in particular sodium hydroxide and potassium hydroxide, sodium carbonate and potassium carbonate and sodium bicarbonate and potassium bicarbonate. These salts are compounds in which the acidic hydrogen is replaced by an agriculturally suitable cation, for example metal salts, in particular alkali metal salts or alkaline earth metal salts, especially sodium salts and potassium salts, or else ammonium salts, salts with organic amines or quaternary ammonium salts.

By forming an adduct with a suitable inorganic or organic acid, such as, for example, mineral acids, such as, for example, HCl, HBr, H₂SO₄ or HNO₃, or organic acids, such as formic acid, acetic acid, propionic acid, oxalic acid or sulfonic acids, at a basic group, such as, for example, amino, alkylamino, dialkylamino, piperidino, morpholino or pyridino, the compounds of the formula (I) are capable of forming salts. In this case, the salts contain the conjugated base of the acid as anion.

Suitable substituents which are present in deprotonated form, such as, for example, sulfonic acids or carboxylic acids, are capable of forming inner salts with groups which for their part can be protonated, such as amino groups.

Hereinbelow, the compounds of the formula (I) and their salts are, in short, also referred to as “compounds (I)” according to the invention or used according to the invention.

The terms used above and further below are familiar to the person skilled in the art and have in particular the meanings illustrated below:

An inorganic radical is a radical without carbon atoms, preferably halogen, OH and its inorganic salts, where the H is replaced by a cation, for example alkali metal and alkaline earth metal salts, NH₂ and its ammonium salts with (inorganic) acids, for example mineral acids, N₃ (azide), N₂⁺A⁻ (diazonium radical, where A⁻ is an anion), NO, NHOH, NHNH₂, NO₂, S(O)OH (sulfinic acid radical), S(O)₂OH (or, in short, also SO₃H, sulfonic acid radical), —O—SO₂H (sulfite), —O—SO₃H (sulfate), —P(O)(OH)₂ (phosphonic acid radical), —O—P(OH)₃, (phosphate radical) and the hydrated or dehydrated forms of the 6 last-mentioned acid radicals and their (inorganic) salts; the term “inorganic radical” also embraces the hydrogen radical (the hydrogen atom), this radical in the definitions frequently already being a part of the unsubstituted skeleton of an organic radical (for example “unsubstituted phenyl”); here, the term “inorganic radical” does preferably not embrace pseudohalogen groups, such as CN, SCN, organic metal complexes, carbonate or COOH, which, owing to their content of carbon atoms, are better assigned to the organic radicals.

An organic radical is a radical having carbon atoms, it also being possible for this radical to be attached by a heteroatom. It is preferably an optionally substituted hydrocarbon radical or an optionally substituted heterocyclic radical. However, the term also, preferably, embraces acyl radicals, i.e. radicals of organic acids formed by removing an OH group. Acyl radicals also include sulfonic acid ester, phosphonic acid ester and phosphinic acid ester groups, having in each case organic alcohol components (and in this case derived from polybasic acids), or alkylsulfonyl or alkylsulfinyl derived from sulfonic acids and sulfinic acids, respectively.

A hydrocarbon radical is an aliphatic, cycloaliphatic or aromatic monocyclic or, in the case of an optionally substituted hydrocarbon radical, also bicyclic or polycyclic organic radical based on the elements carbon and hydrogen, which includes, for example, the radicals alkyl, alkenyl, alkynyl, cycloalkyl, cycloalkenyl, aryl, phenyl, naphthyl, indanyl, indenyl, etc.; this applies correspondingly to the hydrocarbonoxy radicals or other hydrocarbon radicals attached via heteroatom groups. Unless defined more specifically, the hydrocarbon or hydrocarbonoxy radicals in the above definitions preferably have 1 to 20 carbon atoms, more preferably 1 to 16 carbon atoms and in particular 1 to 12 carbon atoms.

In the carbon skeleton, the hydrocarbon radicals and the specific radicals alkyl, alkoxy, haloalkyl, haloalkoxy, alkylamino and alkylthio and the corresponding unsaturated and/or substituted radicals can in each case be straight-chain or branched.

The term “(C₁-C₄)-alkyl” is a short notation for open-chain alkyl having 1 to 4 carbon atoms corresponding to the stated range of carbon atoms, i.e. it includes the radicals methyl, ethyl, 1-propyl, 2-propyl, 1-butyl, 2-butyl, 2-methylpropyl and tert-butyl. Correspondingly, general alkyl radicals having a wider stated range of carbon atoms, for example “(C₁-C₆)-alkyl”, also include straight-chain or branched alkyl radicals having a larger number of carbon atoms, i.e. in the example also the alkyl radicals having 5 and 6 carbon atoms.

Unless specifically indicated, the lower carbon skeletons, for example those having 1 to 6 carbon atoms or, in the case of unsaturated groups, having 2 to 6 carbon atoms, are preferred for the hydrocarbon radicals such as alkyl, alkenyl and alkynyl radicals, including in composite radicals. Alkyl radicals, including in the composite meanings, such as alkoxy, haloalkyl, etc., are, for example, methyl, ethyl, n- or isopropyl, n-, iso, t- or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the meanings of the possible unsaturated radicals which correspond to the alkyl radicals; alkenyl is, for example, vinyl, allyl, 1-methyl-2-propenyl, 2-methyl-2-propenyl, 2-butenyl, pentenyl, 2-methylpentenyl or hexenyl, preferably allyl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl. (C₂-C₆)-alkynyl is, for example, ethynyl, propargyl, 1-methyl-2-propynyl, 2-methyl-2-propynyl, 2-butynyl, 2-pentynyl or 2-hexynyl, preferably propargyl, but-2-yn-1-yl, but-3-yn-1-yl or 1-methylbut-3-yn-1-yl.

Alkylidene, including, for example, in the form (C₁-C₁₀)-alkylidene, is the radical of a straight-chain or branched alkane which is attached via a double bond, where the position of the point of attachment has not yet been fixed. The only possible positions in the case of a branched alkane are, of course, positions in which two hydrogen atoms may be replaced by the double bond; radicals are, for example, ═CH₂, ═CH—CH₃, ═C(CH₃)—CH₃, ═C(CH₃)—C₂H₅ or ═C(C₂H₅)—C₂H₅.

Cycloalkyl is a carbocyclic saturated ring system having preferably 3-8 carbon atoms, for example cyclopropyl, cyclobutyl, cyclopentyl or cyclohexyl. Substituted cycloalkyl embraces cyclic systems having substituents, substituents having a double bond at the cycloalkyl radical, for example an alkylidene group, such as methylidene, also being included. Substituted cycloalkyl also embraces polycyclic aliphatic systems, such as, for example, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl.

Cycloalkenyl is a carbocyclic, non-aromatic, partially unsaturated ring system having preferably 4-8 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. The illustrations given for substituted cycloalkyl apply correspondingly to substituted cycloalkenyl.

The term “halogen” denotes, for example, fluorine, chlorine, bromine or iodine. Haloalkyl, haloalkenyl and haloalkynyl are alkyl, alkenyl and alkynyl, respectively, which are partially or fully substituted by identical or different halogen atoms, preferably from the group consisting of fluorine, chlorine and bromine, in particular from the group consisting of fluorine and chlorine, for example monohaloalkyl, such as CH₂CH₂Cl, CH₂CH₂F, CH₂ClCH₃, CH₂FCH₃, CH₂Cl, CH₂F; perhaloalkyl such as CCl₃ or CF₃ or CF₃CF₂; polyhaloalkyl, such as CHF₂, CH₂F, CH₂FCHCl, CHCl₂, CF₂CF₂H, CH₂CF₃, CH₂ClCH₃, CH₂FCH₃; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

Aryl is a mono-, bi- or polycyclic aromatic system having preferably 6 to 14, in particular 6 to 12, carbon atoms, for example phenyl, naphthyl, tetrahydronaphthyl, indenyl, indanyl, pentalenyl, fluorenyl, biphenylyl and the like, preferably phenyl.

A heterocyclic radical or ring (heterocyclyl) contains at least one heterocyclic ring which is saturated, unsaturated or heteroaromatic and which, in the general substituted case, may be fused with other carbocyclic or heterocyclic rings; unless defined otherwise, the heterocyclic ring preferably contains 3 to 9 ring atoms, in particular 3 to 6 ring atoms, and one or more, preferably 1 to 4, in particular 1, 2 or 3, heteroatoms in the heterocyclic ring, preferably from the group consisting of N, O and S, where, however, two oxygen atoms must not be directly adjacent and at least one carbon atom has to be present in the ring, for example a radical of thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, morpholine, piperazine, oxetane, oxirane, pyrrolidine, oxazoline, tetrahydrofuran, tetrahydropyran, 1,3-dioxolane, 1,3- and 1,4-dioxane, isoxazolidine or thiazolidine.

From among the groups mentioned above under “heterocyclyl”, “heteroaryl” refers in each case to the completely unsaturated aromatic heterocyclic compounds, for example pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine or pyridazine.

More preferably, heterocyclyl is a partially or fully hydrogenated heterocyclic radical having a heteroatom from the group consisting of N, O and S, for example oxiranyl, oxetanyl, oxolanyl (=tetrahydrofuryl), oxanyl, pyrrolinyl, pyrrolidinyl or piperidinyl. More preferably, it is a partially or fully hydrogenated heterocyclic radical having 2 heteroatoms from the group consisting of N, O and S, for example oxazolinyl, thiazolinyl, piperazinyl, 1,3-dioxolanyl, 1,3- and 1,4-dioxanyl, oxazolinyl, isoxazolinyl, oxazolidinyl, isoxazolidinyl and morpholinyl.

If it is a partially or fully saturated nitrogen heterocycle, this may be attached to the remainder of the molecule either via carbon or via the nitrogen.

Heterocyclyl is preferably an aliphatic saturated or unsaturated, in particular saturated, heterocyclyl radical having 3 to 7, in particular 3 to 6, ring atoms, or a heteroaromatic radical having 5 or 6 ring atoms. Heterocyclyl preferably contains hetero ring atoms from the group consisting of N, O and S.

Preferred examples of heterocyclyl are heterocyclic radicals having 3 to 6 ring atoms, from the group consisting of pyridyl, thienyl, furyl, pyrrolyl, oxiranyl, 2-oxetanyl, 3-oxetanyl, oxolanyl (=tetrahydrofuryl), pyrrolidinyl, piperidinyl, in particular oxiranyl, 2-oxetanyl, 3-oxetanyl or oxolanyl, and heterocyclic radicals having two or three heteroatoms, for example pyrimidinyl, pyridazinyl, pyrazinyl, triazinyl, thienyl, thiazolyl, thiadiazolyl, oxazolyl, isoxazolyl, pyrazolyl, triazolyl, piperazinyl, dioxolanyl, dioxanyl, oxazolinyl, isoxazolinyl, oxazolidinyl, isoxazolidinyl or morpholinyl.

If a skeleton is substituted “by one or more radicals” from a list of radicals (=group) or a generically defined group of radicals, this includes in each case the simultaneous substitution by a plurality of identical and/or structurally different radicals.

Suitable substituents for a substituted heterocyclic radical are the substituents mentioned further below, and additionally also oxo. In this case, the oxo group as a substituent on a ring carbon atom is, for example, a carbonyl group in the heterocyclic ring. This preferably also embraces lactones and lactams. The oxo group may also be present at the hetero ring atoms, which may exist in various oxidation states, for example in the case of nitrogen and sulfur, and then form, for example, the divalent groups —N(O)—, —S(O)— (also in short SO) and —S(O)₂— (also in short SO₂) in the heterocyclic ring.

In a heterocyclic ring, substituents different from the oxo group may also be attached to a heteroatom, for example a nitrogen atom, if a hydrogen atom at the nitrogen atom of the skeleton is replaced. Also possible in the case of the nitrogen atom and also other heteroatoms, such as, for example, the sulfur atom, is a further substitution with formation of quaternary ammonium compounds or sulfonium compounds.

Substituted radicals, such as a substituted alkyl, alkenyl, alkynyl, cycloalkyl, cycloalkenyl, aryl, phenyl, benzyl, heterocyclyl and heteroaryl radical, are, for example, substituted radicals derived from an unsubstituted skeleton, the substituents being, for example, one or more, preferably 1, 2 or 3, radicals from the group consisting of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, trialkylsilyl and optionally substituted cycloalkyl, optionally substituted aryl, optionally substituted heterocyclyl, where each of the last-mentioned cyclic groups may also be attached via heteroatoms or divalent functional groups as in the alkyl radicals mentioned, and alkylsulfinyl, alkylsulfonyl and, in the case of cyclic radicals (=“cyclic skeleton”), also alkyl, haloalkyl, alkylthioalkyl, alkoxyalkyl, optionally substituted mono- and dialkylaminoalkyl and hydroxyalkyl; the term “substituted radicals”, such as substituted alkyl, etc., includes as substituents, in addition to the saturated hydrocarbon-containing radicals mentioned, the corresponding unsaturated aliphatic and aromatic radicals, such as optionally substituted alkenyl, alkynyl, alkenyloxy, alkynyloxy, phenyl, phenoxy etc. In the case of substituted cyclic radicals having aliphatic moieties in the ring, this also embraces cyclic systems having substituents which are attached to the ring via a double bond, for example substituted by an alkylidene group, such as methylidene or ethylidene, or an oxo group, imino group or substituted imino group.

The substituents mentioned by way of example (“first substituent level”) can, if they contain hydrocarbon-containing moieties, be, if appropriate, substituted further in the moieties (“second substituent level”), for example by one of the substituents as defined for the first substituent level. Corresponding further substituent levels are possible. The term “substituted radical” preferably embraces only one or two substituent levels.

Preferred substituents for the substituent levels are, for example, amino, hydroxyl, halogen, nitro, cyano, mercapto, carboxyl, carboxamide, SF₅, aminosulfonyl, alkyl, cycloalkyl, alkenyl, cycloalkenyl, alkynyl, monoalkylamino, dialkylamino, N-alkanoylamino, alkoxy, alkenyloxy, alkynyloxy, cycloalkoxy, cycloalkenyloxy, alkoxycarbonyl, alkenyloxycarbonyl, alkynyloxycarbonyl, aryloxycarbonyl, alkanoyl, alkenylcarbonyl, alkynylcarbonyl, arylcarbonyl, alkylthio, cycloalkylthio, alkenylthio, cycloalkenylthio, alkynylthio, alkylsulfinyl, alkylsulfonyl, monoalkylaminosulfonyl, dialkylaminosulfonyl, N-alkylaminocarbonyl, N,N-dialkylaminocarbonyl, N-alkanoylaminocarbonyl, N-alkanoyl-N-alkylaminocarbonyl, aryl, aryloxy, benzyl, benzyloxy, benzylthio, arylthio, arylamino, benzylamino, heterocyclyl and trialkylsilyl.

In the case of radicals having carbon atoms, preference is given to those having 1 to 6 carbon atoms, preferably 1 to 4 carbon atoms, in particular 1 or 2 carbon atoms. Preference is generally given to substituents selected from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano. Here, particular preference is given to the substituents methyl, methoxy, fluorine and chlorine.

Substituted amino, such as mono- or disubstituted amino, denotes a radical from the group of the substituted amino radicals which are N-substituted, for example, by one or two identical or different radicals selected from the group consisting of alkyl, alkoxy, acyl and aryl; preferably mono- and dialkylamino, mono- and diarylamino, acylamino, N-alkyl-N-arylamino, N-alkyl-N-acylamino and saturated N-heterocycles; here, preference is given to alkyl radicals having 1 to 4 carbon atoms; aryl is preferably phenyl or substituted phenyl; for acyl, the definition given further down applies, preference is given to (C₁-C₄)-alkanoyl. This applies correspondingly to substituted hydroxylamino or hydrazino.

Substituted amino also includes quaternary ammonium compounds (salts) having four organic substituents at the nitrogen atom.

Optionally substituted phenyl is preferably phenyl which is unsubstituted or mono- or polysubstituted, preferably up to trisubstituted, by identical or different radicals from the group consisting of halogen, (C₁-C₄)-alkyl, (C₁-C₄)-alkoxy, (C₁-C₄)-haloalkyl, (C₁-C₄)-haloalkoxy and nitro, for example o-, m- and p-tolyl, dimethylphenyls, 2-, 3- and 4-chlorophenyl, 2-, 3- and 4-fluorophenyl, 2-, 3- and 4-trifluoromethyl- and -trichloromethylphenyl, 2,4-, 3,5-, 2,5- and 2,3-dichlorophenyl, o-, m- and p-methoxyphenyl.

Optionally substituted cycloalkyl is preferably cycloalkyl which is unsubstituted or mono- or polysubstituted, preferably up to trisubstituted, by identical or different radicals from the group consisting of halogen, (C₁-C₄)-alkyl, (C₁-C₄)-alkoxy, (C₁-C₄)-haloalkyl and (C₁-C₄)-haloalkoxy, in particular by one or two (C₁-C₄)-alkyl radicals.

Optionally substituted heterocyclyl is preferably heterocyclyl which is unsubstituted or mono- or polysubstituted, preferably up to trisubstituted, by identical or different radicals from the group consisting of halogen, (C₁-C₄)-alkyl, (C₁-C₄)-alkoxy, (C₁-C₄)-haloalkyl, (C₁-C₄)-haloalkoxy, nitro and oxo, in particular mono- or polysubstituted by radicals from the group consisting of halogen, (C₁-C₄)-alkyl, (C₁-C₄)-alkoxy, (C₁-C₄)-haloalkyl and oxo, very particularly preferably substituted by one or two (C₁-C₄)-alkyl radicals.

Acyl denotes a radical of an organic acid which, formally, is formed by removing a hydroxyl group from the acid function, it also being possible for the organic radical in the acid to be attached to the acid function via a heteroatom. Examples of acyl are the radical —CO—R of a carboxylic acid HO—CO—R and radicals of acids derived therefrom, such as thiocarboxylic acid, unsubstituted or N-substituted iminocarboxylic acids, the radical of carbonic acid monoesters, N-substituted carbamic acid, sulfonic acids, sulfinic acids, N-substituted sulfonamido acids, phosphonic acids, phosphinic acids.

Acyl denotes, for example, formyl, alkylcarbonyl such as [(C₁-C₄)-alkyl]carbonyl, phenylcarbonyl, alkyloxycarbonyl, phenyloxycarbonyl, benzyloxycarbonyl, alkylsulfonyl, alkylsulfinyl, N-alkyl-1-iminoalkyl, N-alkyl- and N,N-dialkylcarbamoyl and other radicals of organic acids. Here, the radicals may in each case be substituted further in the alkyl or phenyl moiety, for example in the alkyl moiety by one or more radicals selected from the group consisting of halogen, alkoxy, phenyl and phenoxy; examples of substituents in the phenyl moiety are the substituents which have already been mentioned further above in a general manner for substituted phenyl.

Acyl denotes preferably an acyl radical in the narrower sense, i.e. a radical of an organic acid where the acid group is attached directly to the carbon atom of an organic radical, for example alkanoyl, such as formyl and acetyl, aroyl, such as phenylcarbonyl, and other radicals of saturated or unsaturated organic acids.

“Aroyl” denotes an aryl radical as defined above which is attached via a carbonyl group, for example the benzoyl group.

If a general radical is defined as “hydrogen”, this means a hydrogen atom.

The “yl-position” of a radical denotes its point of attachment.

In accordance with the general definitions:

“(C₁-C₆)-alkyl” is a methyl, ethyl, propyl, isopropyl, 1-butyl, 2-butyl, 2-methylpropyl or tert-butyl radical;

“(C₁-C₁₀)-alkyl” thus includes the alkyl radicals mentioned above, and also isomeric pentyl radicals, such as n-pentyl, 1,1-dimethylpropyl or 2-methylbutyl, isomeric hexyl, heptyl, octyl, nonyl or decyl radicals.

Accordingly, “(C₂-C₄)-alkenyl” denotes, for example, the vinyl, allyl, 2-methyl-2-propen-1-yl-, 2- or 3-buten-1-yl group,

accordingly, “(C₃-C₁₀)-alkenyl” denotes, for example, the allyl, 2-methyl-2-propen-1-yl, 2- or 3-buten-1-yl, pentenyl, 2-methylpentenyl, hexenyl, heptenyl, octenyl, nonenyl or decenyl group.

“(C₂-C₄)-Alkynyl” denotes, for example, the ethynyl, propargyl or 2-butyn-1-yl group,

“(C₃-C₁₀)-alkynyl” denotes, for example, the propargyl, 2-butyn-1-yl, 2-pentyn-1-yl, 2-methylpentyn-3-yl, hexynyl, heptynyl, octynyl, nonynyl or the decynyl group.

If the carbon chain of an alkyl radical is interrupted by more than one oxygen atom, this means that two oxygen atoms must not be directly adjacent.

“(C₃-C₆)-Cycloalkyl” denotes the cyclopropyl, cyclobutyl, cyclopentyl or cyclohexyl radical,

“(C₃-C₁₀)-cycloalkyl” denotes monocyclic alkyl radicals, such as the cyclopropyl, cyclobutyl, cyclopentyl, cyclohexyl, cycloheptyl, cyclooctyl or cyclodecyl radical, denotes bicyclic alkyl radicals, such as the norbornyl or bicyclo[2.2.2]octyl radical, or denotes fused systems, such as the decahydronaphthyl radical.

“(C₄-C₁₀)-Cycloalkenyl” denotes monocyclic cycloalkylene radicals, such as the cyclobutenyl, cyclopentenyl, cyclohexenyl, cycloheptenyl, cyclooctenyl or cyclodecenyl radical, denotes bicyclic alkyl radicals, such as the norbornenyl or bicyclo[2.2.2]octenyl radical, or denotes fused systems, such as the tetra-, hexa- or octahydronaphthyl radical.

“(C₁-C₄)-Alkoxy” and “(C₁-C₁₀)-alkoxy” are alkoxy groups whose hydrocarbon radicals have the meanings given under the terms “(C₁-C₄)-alkyl” and “(C₁-C₁₀)-alkyl”.

“(C₁-C₄)-Alkoxy-(C₁-C₄)-alkoxy” is an alkoxy group as defined above which is substituted by a further alkoxy group, such as the ethoxymethoxy, methoxymethoxy, 1-methoxyethoxy, 1-ethoxyethoxy or the 1-methoxypropoxy group.

“(C₃-C₁₀)-Alkenyloxy”, “(C₃-C₁₀)-alkynyloxy”, “(C₃-C₁₀)-cycloalkoxy” and “(C₄-C₁₀)-cycloalkenyloxy” are ether groups whose hydrocarbon radicals have the meanings given under the terms “(C₃-C₁₀)-alkenyl”, “(C₃-C₁₀)-alkynyl”, “(C₃-C₁₀)-cycloalkyl” and “(C₄-C₁₀)-cycloalkenyl”.

“(C₃-C₁₀)-Cycloalkyl-(C₁-C₄)-alkoxy” denotes, for example, the cyclopropylmethoxy, cyclopropylethoxy, cyclobutylmethoxy, cyclopentylmethoxy, cyclohexylmethoxy or the cyclohexylethoxy group.

“(C₄-C₁₀)-Cycloalkenyl-(C₁-C₄)-alkoxy” denotes, for example, the cyclobutenylmethoxy, cyclopentenylmethoxy, cyclohexenylmethoxy or the cyclohexenylethoxy group.

“(C₃-C₁₀)-Cycloalkyl-(C₃-C₄)-alkenyloxy” denotes, for example, the cyclopropylallyloxy, cyclobutylallyloxy or the cyclopentylallyloxy group.

“(C₄-C₁₀)-Cycloalkenyl-(C₃-C₄)-alkenyloxy” denotes, for example, the cyclobutenylallyloxy or the cyclopentenylallyloxy group.

“(C₁-C₄)-Alkyl-(C₃-C₁₀)-cycloalkoxy” denotes, for example, the methylcyclopentyloxy, ethylcyclopentyloxy, methylcyclohexyloxy or the ethylcyclohexyloxy group.

“(C₂-C₄)-Alkenyl-(C₃-C₁₀)-cycloalkoxy” denotes, for example, the vinylcyclopentyloxy, allylcyclopentyloxy, vinylcyclohexyloxy or the allylcyclohexyloxy group.

“(C₂-C₄)-Alkynyl-(C₃-C₁₀)-cycloalkoxy” denotes, for example, the ethynylcyclopentyloxy, propynylcyclopentyloxy, ethynylcyclohexyloxy or the propynylcyclohexyloxy group.

“(C₁-C₄)-Alkyl-(C₄-C₁₀)-cycloalkenyloxy” denotes, for example, the methylcyclopentenyloxy, ethylcyclopentenyloxy, methylcyclohexenyloxy or the ethylcyclohexenyloxy group.

“(C₂-C₄)-Alkenyl-(C₃-C₁₀)-cycloalkenyloxy” denotes, for example, the vinylcyclopentenyloxy, allylcyclopentenyloxy, vinylcyclohexenyloxy or the allylcyclohexenyloxy group.

“(C₁-C₄)-Alkoxy-(C₃-C₄)-alkenyloxy” denotes, for example, the methoxyallyloxy or the ethoxyallyloxy group.

“(C₁-C₁₀)-Alkanoyl” denotes, for example, the formyl, acetyl, propionyl, butyryl, 2-methylbutyryl, pivaloyl, octanoyl or decanoyl group.

“(C₄-C₁₀)-Cycloalkanoyl” denotes, for example, the cyclobutylcarbonyl, cyclopentylcarbonyl, cyclohexylcarbonyl or the cyclononylcarbonyl group.

“(C₃-C₁₀)-Alkenoyl” denotes, for example, the acryl, methacryl, crotonoyl, dimethylacryl or octenoyl group.

“(C₃-C₁₀)-Alkynoyl” denotes, for example, the propynoyl, butynoyl, hexynoyl or octynoyl group.

“Mono- and di-(C₁-C₄)-alkylcarbamoyl where in the latter group the alkyl groups may also be linked in a cyclic manner to form a three- to eight-membered ring in which optionally one carbon unit may be replaced by oxygen, sulfur or a group S(O), S(O)₂ or NR³ and R³ is (C₁-C₄)-alkyl, (C₁-C₄)-alkanoyl, (C₁-C₄)-alkoxycarbonyl, di-(C₁-C₄)-alkylcarbamoyl or optionally substituted aryl” denotes, for example, the methyl-, ethyl-, propyl-, isopropyl-, butyl- or tert-butylcarbamoyl group or the dimethyl-, diethyl-, methylethyl- or diisopropylcarbamoyl group, but also cyclic derivatives, such as the pyrrolidino-, morpholino-, thiomorpholino-, piperidino-, N-methyl- or acetyl-piperazinocarbamoyl group.

“Mono- or di-(C₃-C₁₀)-cycloalkylcarbamoyl” denotes, for example, the cyclopropyl-, cyclobutyl-, cyclopentyl- or cyclohexylcarbamoyl group or the dicyclopropyl-, dicyclobutyl-, dicyclopentyl- or dicyclohexylcarbamoyl group.

“(C₁-C₁₀)-Alkoxycarbonyl” denotes, for example, the methoxycarbonyl, ethoxycarbonyl, propoxycarbonyl, isopropoxycarbonyl, butoxycarbonyl, isobutoxycarbonyl, sec-butoxycarbonyl or the tert-butoxycarbonyl group.

“(C₃-C₁₀)-Cycloalkoxycarbonyl” denotes, for example, the cyclopropoxycarbonyl, cyclobutoxycarbonyl, cyclopentyloxycarbonyl or the cyclohexyloxycarbonyl group.

“(C₁-C₁₀)-Alkanoyloxy” denotes, for example, the acetoxy, propionyloxy, butanoyloxy or the pivaloyloxy group.

“(C₄-C₁₀)-Cycloalkanoyloxy” denotes, for example, the cyclopropylcarbonyloxy, cyclobutylcarbonyloxy, cyclopentylcarbonyloxy or the cyclohexylcarbonyloxy group.

“(C₁-C₁₀)-Alkoxycarbonyloxy” denotes a carbonate group, such as, for example, the methoxy-, ethoxy-, propoxy-, isopropoxy-, butoxy- or tert-butoxycarbonyloxy group.

“(C₁-C₁₀)-Alkylaminocarbonyloxy” denotes a carbamate group, such as, for example, the methyl-, ethyl-, propyl-, isopropyl-, butyl- or tert-butylaminocarbonyloxy group.

“Di-(C₁-C₁₀)-alkylaminocarbonyloxy, where in the latter group the alkyl groups may also be linked in a cyclic manner to form a three- to eight-membered ring in which optionally one carbon unit may be replaced by oxygen, sulfur or a group S(O), S(O)₂ or NR³ and R³ is (C₁-C₄)-alkyl, (C₁-C₄)-alkanoyl, (C₁-C₄)-alkoxycarbonyl, di-(C₁-C₄)-alkylcarbamoyl or optionally substituted aryl” denotes a carbamate group, such as, for example, the dimethyl-, diethyl-, methylethyl-, dibutyl-, pyrrolidino-, piperidino-, morpholino-, acetylpiperazino- or N-methylpiperazinocarbonyloxy group.

“(C₁-C₁₀)-Alkylsulfonylamino” denotes, for example, a methyl-, ethyl-, propyl-, isopropyl-, butyl-, isobutyl-, tert-butyl-, octyl- or decylsulfonylamino group.

“(C₁-C₁₀)-Alkanoylamino” denotes, for example, the formylamino, acetylamino, propionylamino, isopropionylamino, butanoylamino or the pivaloylamino group.

“(C₃-C₁₀)-Alkenoylamino” denotes, for example, the acrylamino, methacrylamino, dimethylacrylamino or the crotonylamino group.

“(C₄-C₁₀)-Cycloalkanoylamino” denotes, for example, the cyclopropanoylamino, cyclobutanoylamino, cyclopentanoylamino or the cyclohexanoylamino group.

“(C₃-C₁₀)-Cycloalkyl-(C₁-C₄)-alkanoylamino” denotes, for example, the cyclopropylacetylamino or the cyclopentylacetylamino group.

“Mono- and di-(C₁-C₁₀)-alkylaminocarbonylamino, where in the latter group the alkyl groups may also be linked in a cyclic manner to form a three- to eight-membered ring in which optionally one carbon unit may be replaced by oxygen, sulfur or a group S(O), S(O)₂ or NR³ and R³ is (C₁-C₄)-alkyl, (C₁-C₄)-alkanoyl, (C₁-C₄)-alkoxycarbonyl, di-(C₁-C₄)-alkylcarbamoyl or optionally substituted aryl” denotes a urea group, such as, for example, the methylamino-, dimethylamino-, ethylamino-, methylethylamino-, piperidino-, morpholino- or acetylpiperazinocarbonylamino group.

“(C₁-C₁₀)-Alkoxycarbonylamino” denotes a urethane group, such as, for example, the methoxy-, ethoxy-, propoxy-, isopropoxy-, butoxy- or tert-butoxycarbonylamino group.

“(C₁-C₁₀)-Alkylthio” denotes an alkylthio group whose hydrocarbon radical has the meaning given under the term “(C₁-C₁₀)-alkyl”.

“(C₃-C₁₀)-Alkenylthio” denotes an alkenylthio group whose hydrocarbon radical has the meaning given under the term “(C₃-C₁₀)-alkenyl”.

“(C₃-C₁₀)-Alkynylthio” denotes an alkynylthio group whose hydrocarbon radical has the meaning given under the term “(C₃-C₁₀)-alkynyl”.

“(C₃-C₁₀)-Cycloalkylthio” denotes a cycloalkylthio group whose hydrocarbon radical has the meaning given under the term “(C₃-C₁₀)-cycloalkyl”.

“(C₄-C₁₀)-Cycloalkenylthio” denotes a cycloalkenylthio group whose hydrocarbon radical has the meaning given under the term “(C₄-C₁₀)-cycloalkenyl”.

“(C₃-C₁₀)-Cycloalkyl-(C₁-C₄)-alkylthio” denotes, for example, the cyclopropylmethylthio, cyclopropylethylthio, cyclopentylmethylthio or the cyclohexylmethylthio group.

“(C₄-C₁₀)-Cycloalkenyl-(C₁-C₄)-alkylthio” denotes, for example, the cyclopentenylmethylthio or the cyclohexenylmethylthio group.

“(C₃-C₁₀)-Cycloalkyl-(C₃-C₄)-alkenylthio” denotes, for example, the cyclopropylallylthio, cyclopentylallylthio or the cyclohexylallylthio group.

“(C₄-C₁₀)-Cycloalkenyl-(C₃-C₄)-alkenylthio” denotes, for example, the cyclopentenylallylthio or the cyclohexenylallylthio group.

“(C₁-C₄)-Alkyl-(C₃-C₁₀)-cycloalkylthio” denotes, for example, the methylcyclopentylthio or the methylcyclohexylthio group.

“(C₁-C₄)-Alkyl-(C₄-C₁₀)-cycloalkenylthio” denotes, for example, the methylcyclopentenylthio or the methylcyclohexenylthio group.

“(C₂-C₄)-Alkenyl-(C₃-C₁₀)-cycloalkylthio” denotes, for example, the vinylcyclopentylthio, allylcyclopentylthio, vinylcyclohexylthio or the allylcyclohexylthio group.

“(C₂-C₄)-Alkynyl-(C₃-C₁₀)-cycloalkylthio” denotes, for example, the ethynylcyclopentylthio, propargylcyclopentylthio, ethynylcyclohexylthio or the propargylcyclohexylthio group.

“(C₁-C₄)-Alkyl-(C₄-C₁₀)-cycloalkenylthio” denotes, for example, the methylcyclopentenylthio or the methylcyclohexenylthio group.

“(C₂-C₄)-Alkenyl-(C₄-C₈)-cycloalkenylthio” denotes, for example, the allylcyclopentenylthio or the allylcyclohexenylthio group.

“(C₁-C₁₀)-Alkylsulfinyl” denotes, for example, the methyl-, ethyl-, propyl-, isopropyl-, butyl-, isobutyl-, sec-butyl-, tert-butyl- or octylsulfinyl group.

“(C₃-C₁₀)-Alkenylsulfinyl” denotes, for example, the allyl-, methylallyl-, butenyl- or octenylsulfenyl group.

“(C₃-C₁₀)-Alkynylsulfinyl” denotes, for example, the propargyl-, butynyl- or octynylsulfinyl group.

“(C₃-C₁₀)-Cycloalkylsulfinyl” denotes a cycloalkylsulfinyl group whose hydrocarbon radical has the meaning given under the term “(C₃-C₁₀)-cycloalkyl”.

“(C₄-C₁₀)-Cycloalkenylsulfinyl” denotes a cycloalkenylsulfinyl group whose hydrocarbon radical has the meaning given under the term “(C₄-C₁₀)-cycloalkenyl”.

“(C₃-C₁₀)-Cycloalkyl-(C₁-C₄)-alkylsulfinyl” denotes, for example, the cyclopropylmethylsulfinyl, cyclopropylethylsulfinyl, cyclopentylmethylsulfinyl, or the cyclohexylmethylsulfinyl group.

“(C₄-C₁₀)-Cycloalkenyl-(C₁-C₄)-alkylsulfinyl” denotes, for example, the cyclopentenylmethylsulfinyl or the cyclohexenylmethylsulfinyl group.

“(C₃-C₁₀)-Cycloalkyl-(C₃-C₄)-alkenylsulfinyl” denotes, for example, the cyclopropylallylsulfinyl, cyclopentylallylsulfinyl or the cyclohexylallylsulfinyl group.

“(C₄-C₁₀)-Cycloalkenyl-(C₃-C₄)-alkenylsulfinyl” denotes, for example, the cyclopentenylallylsulfinyl or the cyclohexenylallylsulfinyl group.

“(C₁-C₄)-Alkyl-(C₃-C₁₀)-cycloalkylsulfinyl” denotes, for example, the methylcyclopentylsulfinyl or the methylcyclohexylsulfinyl group.

“(C₁-C₈)-Alkyl-(C₄-C₁₀)-cycloalkenylsulfinyl” denotes, for example, the methylcyclopentenylsulfinyl or the methylcyclohexenylsulfinyl group.

“(C₂-C₄)-Alkenyl-(C₃-C₁₀)-cycloalkylsulfinyl” denotes, for example, the vinylcyclopentylsulfinyl, allylcyclopentylsulfinyl, vinylcyclohexylsulfinyl or the allylcyclohexylsulfinyl group.

“(C₂-C₄)-Alkynyl-(C₃-C₁₀)-cycloalkylsulfinyl” denotes, for example, the ethynylcyclopentylsulfinyl, propargylcyclopentylsulfinyl, ethynylcyclohexylsulfinyl or the propargylcyclohexylsulfinyl group.

“(C₂-C₄)-Alkenyl-(C₄-C₁₀)-cycloalkenylsulfinyl” denotes, for example, the vinylcyclopentenylsulfinyl, allylcyclopentenylsulfinyl, vinylcyclohexenylsulfinyl or the allylcyclohexenylsulfinyl group.

“(C₂-C₄)-Alkynyl-(C₄-C₁₀)-cycloalkenylsulfinyl” denotes, for example, the ethynylcyclopentenylsulfinyl, propargylcyclopentenylsulfinyl, ethynylcyclohexenylsulfinyl or the propargylcyclohexenylsulfinyl group.

“(C₁-C₁₀)-Alkylsulfonyl” denotes, for example, the methyl-, ethyl-, propyl-, isopropyl-, butyl-, isobutyl-, sec-butyl-, tert-butyl- or octylsulfonyl group.

“(C₃-C₁₀)-Alkenylsulfonyl” denotes, for example, the allyl-, methylallyl-, butenyl- or octenylsulfonyl group.

“(C₃-C₁₀)-Alkynylsulfonyl” denotes, for example, the propargyl-, butynyl- or octynyl-sulfonyl group.

“(C₃-C₁₀)-Cycloalkylsulfonyl” denotes a cycloalkylsulfonyl group whose hydrocarbon radical has the meaning given under the term “(C₃-C₁₀)-cycloalkyl”.

“(C₄-C₁₀)-Cycloalkenylsulfonyl” denotes a cycloalkenylsulfonyl group whose hydrocarbon radical has the meaning given under the term “(C₄-C₁₀)-cycloalkenyl”.

“(C₃-C₁₀)-Cycloalkyl-(C₁-C₄)-alkylsulfonyl” denotes, for example, the cyclopropylmethylsulfonyl, cyclopropylethylsulfonyl, cyclopentylmethylsulfonyl or the cyclohexylmethylsulfonyl group.

“(C₄-C₁₀)-Cycloalkenyl-(C₁-C₄)-alkylsulfonyl” denotes, for example, the cyclopentenylmethylsulfonyl or the cyclohexenylmethylsulfonyl group.

“(C₃-C₁₀)-Cycloalkyl-(C₃-C₄)-alkenylsulfonyl” denotes, for example, the cyclopropylallylsulfonyl, cyclopentylallylsulfonyl, or the cyclohexylallylsulfonyl group.

“(C₄-C₁₀)-Cycloalkenyl-(C₃-C₄)-alkenylsulfonyl” denotes, for example, the cyclopentenylallylsulfonyl or the cyclohexenylallylsulfonyl group.

“(C₁-C₄)-Alkyl-(C₃-C₁₀)-cycloalkylsulfonyl” denotes, for example, the methylcyclopentylsulfonyl or the methylcyclohexylsulfonyl group.

“(C₁-C₄)-Alkyl-(C₄-C₁₀)-cycloalkenylsulfonyl” denotes, for example, the methylcyclopentenylsulfonyl or the methylcyclohexenylsulfonyl group.

“(C₂-C₄)-Alkenyl-(C₃-C₁₀)-cycloalkylsulfonyl” denotes, for example, the vinylcyclopentylsulfonyl, allylcyclopentylsulfonyl, vinylcyclohexylsulfonyl, or the allylcyclohexylsulfonyl group.

“(C₂-C₄)-Alkynyl-(C₃-C₁₀)-cycloalkylsulfonyl” denotes, for example, the ethynylcyclopentylsulfonyl, propargylcyclopentylsulfonyl, ethynylcyclohexylsulfonyl or the propargylcyclohexylsulfonyl group.

“(C₂-C₄)-Alkenyl-(C₄-C₁₀)-cycloalkenylsulfonyl” denotes, for example, the vinylcyclopentenylsulfonyl, allylcyclopentenylsulfonyl, vinylcyclohexenylsulfonyl or the allylcyclohexenylsulfonyl group.

“Mono- and di-(C₁-C₁₀)-alkylaminosulfonyl, where in the latter group the alkyl groups may also be linked in a cyclic manner to form a three- to eight-membered ring in which optionally one carbon unit may be replaced by oxygen, sulfur or a group S(O), S(O)₂ or NR³ and R³ is (C₁-C₄)-alkyl, (C₁-C₄)-alkanoyl, (C₁-C₄)-alkoxycarbonyl, di-(C₁-C₄)-alkylcarbamoyl or optionally substituted aryl” denotes, for example, the methyl-, ethyl-, propyl-, isopropyl-, butyl-, tert-butyl- or the octylaminosulfonyl group or the dimethyl-, methylethyl-, diethyl- or the dibutylaminosulfonyl group or the pyrrolidino-, piperidino-, morpholino-, N-methylpiperazino- or the N-acetylpiperazinoaminosulfonyl group;

“(C₁-C₁₀)-alkylamino” denotes an amino group whose hydrocarbon radical has the meaning given under the term “(C₁-C₁₀)-alkyl”.

“(C₃-C₁₀)-Alkenylamino” denotes an amino group whose hydrocarbon radical has the meaning given under the term “(C₁-C₁₀)-alkenyl”.

“(C₃-C₁₀)-Alkynylamino” denotes an amino group whose hydrocarbon radical has the meaning given under the term “(C₃-C₁₀)-alkynyl”.

“(C₃-C₁₀)-Cycloalkylamino” denotes an amino group whose hydrocarbon radical has the meaning given under the term “(C₃-C₁₀)-cycloalkyl”.

“(C₃-C₁₀)-Cycloalkenylamino” denotes an amino group whose hydrocarbon radical has the meaning given under the term “(C₃-C₁₀)-cycloalkenyl”.

“(C₃-C₁₀)-Cycloalkyl-(C₁-C₄)-alkylamino” denotes, for example, the cyclopropylmethylamino, cyclopropylethylamino, cyclopentylmethylamino or the cyclohexylmethylamino group.

“(C₄-C₁₀)-Cycloalkenyl-(C₁-C₄)-alkylamino” denotes, for example, the cyclopentenylmethylamino or the cyclohexenylmethylamino group.

“(C₄-C₁₀)-Cycloalkyl-(C₃-C₄)-alkenylamino” denotes, for example, the cyclopropylallylamino, cyclopentylallylamino or the cyclohexylallylamino group.

“(C₄-C₁₀)-Cycloalkenyl-(C₃-C₄)-alkenylamino” denotes, for example, the cyclopentenylallylamino or the cyclohexenylallylamino group.

“(C₁-C₄)-Alkyl-(C₃-C₁₀)-cycloalkylamino” denotes, for example, the methylcyclopentylamino or the methylcyclohexylamino group.

“(C₁-C₄)-Alkyl-(C₄-C₁₀)-cycloalkenylamino” denotes, for example, the methylcyclopentenylamino or the methylcyclohexenylamino group.

“(C₂-C₄)-Alkenyl-(C₃-C₁₀)-cycloalkylamino” denotes, for example, the vinylcyclopentylamino, allylcyclopentylamino, vinylcyclohexylamino or the allylcyclohexylamino group.

“(C₂-C₄)-Alkynyl-(C₃-C₁₀)-cycloalkylamino” denotes, for example, the ethynylcyclopentylamino, propargylcyclopentylamino, ethynylcyclohexylamino or the propargylcyclohexylamino group.

“(C₂-C₄)-Alkenyl-(C₄-C₁₀)-cycloalkenylamino” denotes, for example, the vinylcyclopentenylamino, allylcyclopentenylamino, vinylcyclohexenylamino or the allylcyclohexenylamino group.

“(C₁-C₁₀)-Trialkylsilyl” denotes a silicon atom which carries three identical or different alkyl radicals according to the above definition.

“Aryloxy” denotes an aryl radical as defined above which is attached via an oxygen atom, for example phenoxy or the naphthyloxy group.

“Arylthio” denotes an aryl radical which is attached via a sulfur atom, for example the phenylthio or the 1- or 2-naphthylthio radical.

“Arylamino” denotes an aryl radical which is attached via a nitrogen atom, for example the anilino or the 1- or 2-naphthylamino radical.

“N-(C₁-C₄)-Alkylarylamino” denotes, for example, the N-methyl- or N-ethylanilino radical.

“Aryl-(C₁-C₄)-alkoxy” denotes an aryl radical which is attached via a (C₁-C₄)-alkoxy group, for example the benzyloxy, phenylethoxy, phenylbutoxy or the naphthylmethoxy radical.

“Aryl-(C₃-C₄)-alkenyloxy” denotes an aryl radical which is attached via a (C₃-C₄)-alkenyloxy group, for example the 1-, 2- or 3-phenylallyloxy radical.

“Aryl-(C₁-C₄)-alkylthio” denotes an aryl radical which is attached via an alkylthio radical, for example benzylthio, naphthylmethylthio or the 1- or 2-phenylethylthio radical.

“Aryl-(C₃-C₄)-alkenylthio” denotes an aryl radical which is attached via a (C₃-C₄)-alkenylthio group, for example the 1-, 2- or 3-phenylallylthio radical.

“Aryl-(C₁-C₄)-alkylamino” denotes an aryl radical which is attached via a (C₁-C₄)-alkylamino group, for example the benzylamino, naphthylamino, the 1- or 2-phenylethylamino or the 3-phenylpropylamino radical.

“N-(C₁-C₄)-Alkyl-N-aryl-(C₁-C₄)-alkylamino” denotes, for example, the N-methyl-N-benzylamino, N-methyl-N-naphthylamino, the N-methyl-N-1- or -2-phenylethylamino or the N-methyl-N-3-phenylpropylamino radical.

“Aryl-(C₃-C₄)-alkenylamino” denotes an aryl radical which is attached via a (C₃-C₄)-alkenylamino group, for example the 1-, 2- or 3-phenylallylamino radical.

“N-(C₁-C₄)-Alkyl-N-aryl-(C₃-C₄)-alkenylamino” denotes, for example, the N-methyl-N-1-, -2- or -3-phenylallylamino radical.

“Arylcarbamoyl” denotes, for example, phenyl- or 1- or 2-naphthylcarbamoyl.

“N-Aryl-N-(C₁-C₄)-alkylcarbamoyl” denotes, for example, N-methyl-N-phenylcarbamoyl or N-methyl-N-1- or -2-naphthylcarbamoyl.

“Aryl-(C₁-C₈)-dialkylsilyl” denotes, for example, a phenyl- or naphthyldimethylsilyl group.

“Diaryl-(C₃-C₄)-alkylsilyl” denotes, for example, a diphenyl-, phenylnaphthyl-, or dinaphthylmethylsilyl group.

“Triarylsilyl” denotes, for example, a triphenyl-, diphenylnaphthyl- or trinaphthylsilyl group.

In particular for reasons of better crop-plant- or useful-plant-protecting action (safener action), better selectivity and/or better preparability, the use according to the invention of compounds of the formula (I) mentioned or salts thereof is of particular interest in which individual radicals have one of the preferred meanings already mentioned or mentioned below, and in particular those which contain a combination of one or more of the preferred meanings already mentioned or mentioned below.

X is preferably oxygen.

Preferably,
(Y)ₙ
   are n substituents Y,
   where each Y independently of the others is a halogen, cyano, nitro, (C₁-C₄)-alkyl, (C₂-C₄)-alkenyl, (C₂-C₄)-alkynyl, (C₁-C₄)-alkoxy, (C₁-C₄)-alkylthio, (C₁-C₄)-alkylsulfinyl, (C₁-C₄)-alkylsulfonyl, (C₁-C₄)-alkoxycarbonyl, (C₁-C₄)-alkylamino or di-[(C₁-C₄)-alkyl]amino radical,
      where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
   (C₃-C₆)-cycloalkyl, (C₄-C₆)-cycloalkenyl, aryl (preferably phenyl) or heterocyclyl (preferably a heterocyclic ring having three to six ring atoms and one to three hetero ring atoms from the group consisting of N, O and S),
      where each of the 4 last-mentioned radicals (or the preferred radicals mentioned in brackets) is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio and in the case of non-aromatic radicals, also oxo, or
   two adjacent groups Y together with the carbon atoms which are directly attached are a four- to six-membered fused-on ring which is carbocyclic or heterocyclic having one or more hetero ring atoms, preferably one to three hetero ring atoms from the group consisting of N, O and S, and which is unsubstituted or substituted by one or more radicals from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio and oxo, and
n is 0, 1, 2, 3 or 4, preferably 0, 1, 2 or 3, in particular 0, 1 or 2, very particularly 0 or 1.

(Y)ₙ are in particular n substituents Y,
where each Y independently of the others is a halogen, cyano, nitro, (C₁-C₄)-alkyl, (C₁-C₄)-haloalkyl, [(C₁-C₄)-alkoxy]-(C₁-C₄)-alkyl, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₅-C₆)-cycloalkenyl radical, optionally substituted aryl (preferably phenyl which is unsubstituted or substituted by one or more radicals from the group consisting of halogen, (C₁-C₄)-alkyl, (C₁-C₄)-haloalkyl and (C₁-C₄)-alkoxy), optionally substituted heterocyclyl (preferably a heterocyclic ring having 3 to 6 ring atoms and one to three hetero ring atoms from the group consisting of N, O and S, which heterocyclyl is unsubstituted or substituted by one or more radicals from the group consisting of halogen, (C₁-C₄)-alkyl, (C₁-C₄)-haloalkyl, (C₁-C₄)-alkoxy and oxo) or (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkoxycarbonyl, (C₁-C₄)-haloalkoxycarbonyl, (C₁-C₄)-alkylamino or di-[(C₁-C₄)-alkyl]amino,
or two adjacent groups Y together with the carbon atoms which are directly attached are a four- to six-membered fused-on ring which is carbocyclic or heterocyclic having one to three hetero ring atoms from the group consisting of N, O and S, where in the latter case one or two heteroatoms are attached to the aromatic ring, and which is unsubstituted or substituted by one or more radicals from the group consisting of halogen, (C₁-C₄)-alkyl and oxo.

More preferably, (Y)ₙ are n substituents Y,
where each Y independently of the others is a halogen, cyano, nitro, (C₁-C₄)-alkyl, (C₁-C₄)-haloalkyl, preferably (C₁-C₄)-fluoroalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl (preferably (C₁-C₄)-fluoroalkylsulfinyl), (C₁-C₄)-alkylsulfonyl, (C₁-C₄)-haloalkylsulfonyl (preferably (C₁-C₄)-fluoroalkylsulfonyl), (C₁-C₄)-alkylamino or di-[(C₁-C₄)-alkyl]amino radical, or
two adjacent groups Y together with the carbon atoms which are directly attached are a four- to six-membered fused-on non-aromatic ring which is carbocyclic or heterocyclic having one or two hetero ring atoms from the group consisting of N and O, where in the latter case one or two heteroatoms are attached to the aromatic ring, and which is unsubstituted or substituted by one or more radicals from the group consisting of halogen and (C₁-C₄)-alkyl.

More preferably, (Y)ₙ are n substituents Y,
where each Y independently of the others is a halogen, (C₁-C₄)-alkyl, (C₁-C₄)-haloalkyl (preferably (C₁-C₄)-fluoroalkyl), (C₁-C₄)-alkoxy or (C₁-C₄)-haloalkoxy (preferably (C₁-C₄)-fluoroalkoxy), (C₁-C₄)-haloalkylthio (preferably (C₁-C₄)-fluoroalkylthio) radical, or
two adjacent groups Y together are the divalent group 2,2-difluoromethylenedioxy (—O—CF₂—O—; 2,2-difluoro-1,3-dioxapropane-1,3-diyl).

Very particularly preferably, the radicals Y are, independently of one another, halogen, cyano, (C₁-C₄)-alkyl, trifluoromethyl, methoxy, difluoromethoxy, trifluoromethoxy, methylthio, trifluoromethylthio or 2,2-difluoromethylenedioxy, in particular fluorine, chlorine, methyl, trifluoromethyl or methoxy.

Preference is also given to the use according to the invention of compounds (I), in which
R¹
   is hydrogen, hydroxyl, amino, (C₁-C₄)-alkylamino, di-[(C₁-C₄)-alkyl]amino, (C₁-C₆)-alkyl, (C₃-C₆)-alkenyl, (C₃-C₆)-alkynyl or (C₁-C₆)-alkoxy,
      where each of the 4 last-mentioned radicals is unsubstituted or substituted by one or more identical or different radicals R^{a} and, including substituents, has 1 to 30 carbon atoms, preferably 1 to 24 carbon atoms, or
   (C₃-C₆)-cycloalkyl, (C₄-C₆)-cycloalkenyl, aryl or heterocyclyl,
      where each of the 4 last-mentioned radicals is unsubstituted or substituted by one or more identical or different radicals R^{b} and, including substituents, has 3 to 30 carbon atoms, preferably 3 to 24 carbon atoms, and
R²
   is hydrogen, (C₁-C₁₀)-alkyl, (C₃-C₁₀)-alkenyl or (C₃-C₁₀)-alkynyl,
      where each of the 3 last-mentioned radicals is unsubstituted or substituted by one or more identical or different radicals R^{c} and, including substituents, has 1 to 30 carbon atoms, preferably 1 to 24 carbon atoms, or
   (C₃-C₆)-cycloalkyl, (C₄-C₆)-cycloalkenyl, aryl or heterocyclyl,
      where each of the 4 last-mentioned radicals is unsubstituted or substituted by one or more identical or different radicals R^{d} and, including substituents, has 3 to 30 carbon atoms, preferably 3 to 24 carbon atoms,
   where in the radicals R¹ and R²
   R^{a} in each case independently of one another are inorganic or organic radicals, preferably radicals from the group consisting of halogen, cyano, nitro and radicals of the formulae -Z^{a}-R^{a}* and R^{cyc-a},
   R^{b} in each case independently of one another are inorganic or organic radicals, preferably radicals from the group consisting of halogen, cyano, nitro and radicals of the formulae -Z^{b}-R^{b}* and R^{b}**,
   R^{c} in each case independently of one another are inorganic or organic radicals, preferably radicals from the group consisting of halogen, cyano, nitro and radicals of the formulae -Z^{c}-R^{c}* and R^{cyc-c},
   R^{d} in each case independently of one another are inorganic or organic radicals, preferably radicals from the group consisting of halogen, cyano, nitro and radicals of the formulae -Z^{d}-R^{d}* and R^{d}**,
   where in the radicals R^{a}, R^{b}, R^{c} and R^{d}
   Z^{a}, Z^{b}, Z^{c} and Z^{d} in each case independently of one another are a divalent group of the formula —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —S—CO—, —CO—S—, —S—CS—, —CS—S—, —O—CO—O—, —NR^{O}—, —O—NR^{O}—, —NR^{O}—O—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″— or else —O—N═CR^{O}— or —CR^{O}═N—O—, where p is in each case the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms (preferably acyl from the group consisting of [(C₁-C₆)-alkyl]carbonyl, [(C₁-C₆)-alkoxy]carbonyl and [(C₁-C₆)-alkyl]sulfonyl) and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
   R^{cyc-a} and R^{cyc-c} are an optionally substituted cyclic hydrocarbon radical having a total of 3 to 24 carbon atoms, preferably 1 to 18 carbon atoms, or an optionally substituted heterocyclic radical having a total of 1 to 24 carbon atoms, preferably 1 to 18 carbon atoms, and
   R^{a}*, R^{b}*, R^{c}*, R^{d}*, R^{b}** and R^{c}** are each independently of one another an optionally substituted hydrocarbon radical having a total of 1 to 24 carbon atoms, preferably 1 to 18 carbon atoms, or an optionally substituted heterocyclic radical having a total of 1 to 24 carbon atoms, preferably 1 to 18 carbon atoms, or
R^{a}*, R^{b}*, R^{c}*, R^{d}* are each independently of one another hydrogen (preferably if chemically stable radicals are included).

More preference is also given to the use according to the invention of compounds (I),
where in the radicals R^{a}, R^{b}, R^{c} and R^{d}
Z^{a}, Z^{b}, Z^{c} and Z^{d} are each independently of one another a divalent group of the formula
   —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —S—CO—, —CO—S—, —S—CS—, —CS—S—, —O—CO—O—, —NR^{O}—, —O—NR^{O}—, —NR^{O}—O—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″— or else —O—N═CR^{O}—, where p is in each case the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms (preferably acyl from the group consisting of (C₁-C₆)-alkanoyl, [(C₁-C₆)-alkoxy]carbonyl and (C₁-C₆)-alkylsulfonyl)
   and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl,
   preferably a divalent group of the formula
   —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″—, where p is in each case the integer 0, 1 or 2 and the radicals R^{O} independently of one another are each hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl, in particular each hydrogen or (C₁-C₄)-alkyl, and R′ and R″ independently of one another are (C₁-C₄)-alkyl, phenyl, phenyl-(C₁-C₄)-alkyl or (C₃-C₆)-cycloalkyl,
R^{cyc-a} and R^{cyc-c} are each an optionally substituted cyclic hydrocarbon radical having a total of 3 to 24 carbon atoms, preferably 1 to 18 carbon atoms, or an optionally substituted heterocyclic radical having a total of 1 to 24 carbon atoms, preferably 1 to 18 carbon atoms, and
R^{a}*, R^{b}*, R^{c}*, R^{d}*, R^{b}** and R^{c}** are each independently of one another an optionally substituted hydrocarbon radical having a total of 1 to 24 carbon atoms, preferably 1 to 18 carbon atoms, or an optionally substituted heterocyclic radical having a total of 1 to 24 carbon atoms, preferably 1 to 18 carbon atoms, or
R^{a}*, R^{b}*, R^{c}*, R^{d}* are each independently of one another hydrogen,
preferably
R^{cyc-a} and R^{cyc-c} are each independently of one another (C₃-C₆)-cycloalkyl, (C₄-C₆)-cycloalkenyl, aryl or heterocyclyl, where each of the 4 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of heterocyclyl, also oxo, and
R^{a}*, R^{b}*, R^{c}*, R^{d}*, R^{b}** and R^{c}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoylamino and, in the case of cyclic radicals, also (C₁-C₄)-alkyl, (C₁-C₄)-haloalkyl, (C₁-C₄)-alkoxy-(C₁-C₄)-alkyl and, in the case of heterocyclyl, also oxo, or
R^{a}*, R^{b}*, R^{c}*, R^{d}* are each independently of one another hydrogen.

More preferred is also the use according to the invention of compounds (I) in which
R¹
   is hydrogen, (C₁-C₆)-alkyl, (C₃-C₆)-alkenyl or (C₃-C₆)-alkynyl,
      where each of the 3 last-mentioned radicals is unsubstituted or substituted by one or more identical or different radicals R^{a} and, including substituents, has 1 to 24 carbon atoms, preferably 1 to 18 carbon atoms, or
   (C₃-C₆)-cycloalkyl or saturated heterocyclyl,
      where each of the 2 last-mentioned radicals is unsubstituted or substituted by one or more identical or different radicals R^{b} and, including substituents, has 3 to 24 carbon atoms, preferably 3 to 18 carbon atoms,
   where
   R^{a} is an inorganic or organic radical, preferably a radical from the group consisting of halogen, cyano, nitro and radicals of the formulae -Z^{a}-R^{a}* and R^{cyc-a},
   R^{b} is an inorganic or organic radical, preferably a radical from the group consisting of halogen, cyano, nitro and radicals of the formulae -Z^{b}-R^{b}* and R^{b}**,
   where in the radicals R^{a} and R^{b}
      Z^{a}, Z^{b}, R^{cyc-a}, R^{a}*, R^{b}* and R^{b}** are as defined above or further below, preferably
   Z^{a}, Z^{b} are, independently of one another, —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″—, where p is in each case the integer 0, 1 or 2 and the radicals R^{O} independently of one another are each hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl, in particular each hydrogen or (C₁-C₄)-alkyl, and R′ and R″ independently of one another are (C₁-C₄)-alkyl, phenyl, phenyl-(C₁-C₄)-alkyl or (C₃-C₆)— cycloalkyl, in particular (C₁-C₄)-alkyl,
   R^{cyc-a} is (C₃-C₆)-cycloalkyl, (C₄-C₆)-cycloalkenyl, phenyl, saturated heterocyclyl, unsaturated non-aromatic heterocyclyl or heteroaryl, where each of the 6 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of saturated or unsaturated non-aromatic heterocyclyl, also oxo, and
      in particular
   R^{cyc-a} is (C₃-C₆)-cycloalkyl, phenyl, saturated heterocyclyl having 3 to 6 ring atoms or heteroaryl having 5 or 6 ring atoms, where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio and, in the case of saturated heterocyclyl, also oxo, and
   R^{a}*, R^{b}* and R^{b}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoylamino and, in the case of cyclic radicals, also (C₁-C₄)-alkyl, (C₁-C₄)-haloalkyl, (C₁-C₄)-alkoxy-(C₁-C₄)-alkyl and, in the case of heterocyclyl, also oxo, or
   R^{a}* and R^{b}* are each independently of one another hydrogen,
      in particular
   R^{a}*, R^{b}* and R^{b}** are each independently of one another
      (C₁-C₆)-alkyl, (C₃-C₆)-cycloalkyl, phenyl, saturated heterocyclyl or heteroaryl, where each of the 5 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio and, in the case of saturated heterocyclyl, also oxo, or
   R^{a}* and R^{b}* are each independently of one another hydrogen.

Preference is furthermore given to the use according to the invention of compounds (I) in which
R²
   is (C₁-C₁₀)-alkyl, (C₃-C₁₀)-alkenyl or (C₃-C₁₀)-alkynyl, in particular (C₁-C₆)-alkyl, (C₃-C₆)-alkenyl or (C₃-C₆)-alkynyl,
      where each of the 6 last-mentioned radicals is unsubstituted or substituted by one or more identical or different radicals R^{c} and, including substituents, has 1 to 24 carbon atoms, preferably 1 to 18 carbon atoms, or
   (C₃-C₆)-cycloalkyl, aryl or heterocyclyl, in particular phenyl or heteroaryl,
      where each of the 5 last-mentioned radicals is unsubstituted or substituted by one or more identical or different radicals R^{d} and, including substituents, has 3 to 24 carbon atoms, preferably 3 to 18 carbon atoms,
   where
   R^{c} is an inorganic or organic radical, preferably a radical from the group consisting of halogen, cyano, nitro and radicals of the formulae -Z^{c}-R^{c}* and R^{cyc-c},
   R^{d} is an inorganic or organic radical, preferably a radical from the group consisting of halogen, cyano, nitro and radicals of the formulae -Z^{d}-R^{d}* and R^{d}**,
   where in the radicals R^{c} and R^{d} the radicals or groups
      Z^{c}, Z^{d}, R^{cyc-c}, R^{c}*, R^{d}* and R^{d}** are as defined above or as defined further below,
   preferably
   Z^{c} and Z^{d} are each independently of one another —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″—, where p is in each case the integer 0, 1 or 2 and the radicals R^{O} independently of one another are each hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl, in particular each hydrogen or (C₁-C₄)-alkyl, and R′ and R″ independently of one another are (C₁-C₄)-alkyl, phenyl, phenyl-(C₁-C₄)-alkyl or (C₃-C₆)-cycloalkyl, in particular (C₁-C₄)-alkyl,
   R^{cyc-c} is (C₃-C₆)-cycloalkyl, (C₄-C₆)-cycloalkenyl, phenyl, saturated heterocyclyl, unsaturated non-aromatic heterocyclyl or heteroaryl, where each of the 6 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of saturated or unsaturated non-aromatic heterocyclyl, also oxo, and
   in particular
   R^{cyc-c} is (C₃-C₆)-cycloalkyl, phenyl, saturated heterocyclyl having 3 to 6 ring atoms or heteroaryl having 5 or 6 ring atoms, where each of the 4 last-mentioned radicals is unsubstituted or substituted by one or more radicals from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio and, in the case of saturated heterocyclyl, also oxo, and
   R^{c}*, R^{d}* and R^{d}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoylamino and in the case of cyclic radicals, also (C₁-C₄)-alkyl, (C₁-C₄)-haloalkyl, (C₁-C₄)-alkoxy-(C₁-C₄)alkyl and, in the case of heterocyclyl, also oxo, or
   R^{c}* and R^{d}* are each independently of one another hydrogen,
   in particular
   R^{c}*, R^{d}* and R^{d}** are each independently of one another (C₁-C₆)-alkyl, (C₃-C₆)-cycloalkyl, phenyl, saturated heterocyclyl or heteroaryl, where each of the 5 last-mentioned radicals is unsubstituted or substituted by one or more radicals from the group consisting of halogen, (C₁-C₄)-alkyl, (C₁-C₄)-haloalkyl, (C₁-C₄)-alkoxy, (C₁-C₄)— haloalkoxy, (C₁-C₄)-alkylthio and, in the case of saturated heterocyclyl, also oxo, or
   R^{c}* and R^{d}* are each independently of one another hydrogen.

Preference is given here to the use according to the invention of compounds (I) in which R¹ is one of the optionally substituted (C₁-C₄)-alkyl, (C₂-C₄)-alkenyl or (C₂-C₄)-alkynyl radicals defined above and R² is one of the optionally substituted phenyl and heteroaryl radicals defined above.

Examples of substituents R^{a} and R^{c} with whom the alkyl, alkenyl, alkynyl and alkoxy groups listed under the radicals R¹ and/or R² may optionally be mono- or polysubstituted (where in the case of polysubstitution the substituents may be identical or different) are the following:
halogen, cyano, nitro, hydroxyl, thio, amino or
(C₁-C₁₀)-alkanoyl, (C₃-C₁₀)-alkenoyl, (C₃-C₁₀)-alkynoyl, (C₄-C₁₀)-cycloalkanoyl or
(C₁-C₁₀)-alkoxy, (C₁-C₁₀)-haloalkoxy, (C₁-C₄)-alkoxy-(C₁-C₄)-alkoxy, (C₃-C₁₀)-alkenyloxy, (C₃-C₁₀)-alkynyloxy, (C₃-C₁₀)-cycloalkoxy, (C₄-C₁₀)-cycloalkenyloxy, (C₃-C₁₀)-cycloalkyl-(C₁-C₄)-alkoxy, (C₄-C₁₀)-cycloalkenyl-(C₁-C₄)-alkoxy, (C₃-C₁₀)-cycloalkyl-(C₃-C₄)-alkenyloxy, (C₄-C₁₀)-cycloalkenyl-(C₃-C₄)-alkenyloxy, (C₁-C₄)-alkyl-(C₃-C₁₀)-cycloalkoxy, (C₂-C₄)-alkenyl-(C₃-C₁₀)-cycloalkoxy, (C₂-C₄)-alkynyl-(C₃-C₁₀)-cycloalkoxy, (C₁-C₄)-alkyl-(C₄-C₁₀)-cycloalkenyloxy, (C₂-C₄)-alkenyl-(C₄-C₁₀)-cycloalkenyloxy, (C₁-C₄)-alkoxy-(C₃-C₄)-alkenyloxy or
carbamoyl, mono- or di-[(C₁-C₄)-alkyl]carbamoyl, mono- or di-[(C₃-C₁₀)-cycloalkyl]-carbamoyl, N-(C₁-C₄)-alkoxy-N-(C₁-C₄)-alkylcarbamoyl or
carboxyl, (C₁-C₁₀)-alkoxycarbonyl, (C₃-C₁₀)-cycloalkoxycarbonyl, (C₁-C₁₀)-alkanoyloxy, (C₄-C₁₀)-cycloalkanoyloxy, (C₁-C₁₀)-alkoxycarbonyloxy, [(C₁-C₁₀)-alkyl]aminocarbonyloxy, di-[(C₁-C₁₀)-alkyl]aminocarbonyloxy, or
(C₁-C₁₀)-alkylsulfonylamino, (C₁-C₁₀)-alkanoylamino, (C₃-C₁₀)-alkenoylamino, (C₄-C₁₀)-cycloalkanoylamino, (C₃-C₁₀)-cycloalkyl-(C₁-C₄)-alkanoylamino, mono- or di-[(C₁-C₁₀)-alkyl]aminocarbonylamino, or
[(C₁-C₁₀)-alkoxy]carbonylamino or
the N-(C₁-C₄)-alkyl analogs of the 8 above-mentioned radicals or
(C₁-C₁₀)-alkylthio, (C₁-C₁₀)-haloalkylthio, (C₃-C₁₀)-alkenylthio, (C₃-C₁₀)-alkynylthio, (C₃-C₁₀)-cycloalkylthio, (C₄-C₁₀)-cycloalkenylthio, (C₃-C₁₀)-cycloalkyl-(C₁-C₄)-alkylthio, (C₄-C₁₀)-cycloalkenyl-(C₁-C₄)-alkylthio, (C₃-C₁₀)-cycloalkyl-(C₃-C₄)-alkenylthio, (C₄-C₁₀)-cycloalkenyl-(C₃-C₄)-alkenylthio, (C₁-C₄)-alkyl-(C₃-C₁₀)-cycloalkylthio, (C₂-C₄)-alkenyl-(C₃-C₁₀)-cycloalkylthio, (C₂-C₄)-alkynyl-(C₃-C₁₀)-cycloalkylthio, (C₁-C₄)-alkyl-(C₄-C₁₀)-cycloalkenylthio, (C₂-C₄)-alkenyl-(C₄-C₁₀)-cycloalkenylthio or (C₁-C₁₀)-alkylsulfinyl, (C₁-C₁₀)-haloalkylsulfinyl, (C₃-C₁₀)-alkenylsulfinyl, (C₃-C₁₀)-alkynylsulfinyl, (C₃-C₁₀)-cycloalkylsulfinyl, (C₄-C₁₀)-cycloalkenylsulfinyl, (C₃-C₁₀)-cycloalkyl-(C₁-C₄)-alkylsulfinyl, (C₄-C₁₀)-cycloalkenyl-(C₁-C₄)-alkylsulfinyl, (C₃-C₁₀)-cycloalkyl-(C₃-C₄)-alkenylsulfinyl, (C₄-C₁₀)-cycloalkenyl-(C₃-C₄)-alkenylsulfinyl, (C₁-C₄)-alkyl-(C₃-C₁₀)-cycloalkylsulfinyl, (C₂-C₄)-alkenyl-(C₃-C₁₀)-cycloalkylsulfinyl, (C₂-C₄)-alkynyl-(C₃-C₁₀)-cycloalkylsulfinyl, (C₃-C₄)-alkyl-(C₄-C₁₀)-cycloalkenylsulfinyl, (C₂-C₄)-alkenyl-(C₄-C₁₀)-cycloalkenylsulfinyl, (C₂-C₄)-alkynyl-(C₄-C₁₀)-cycloalkenylsulfinyl or
(C₁-C₁₀)-alkylsulfonyl, (C₁-C₁₀)-haloalkylsulfonyl, (C₃-C₁₀)-alkenylsulfonyl, (C₃-C₁₀)-alkynylsulfonyl, (C₃-C₁₀)-cycloalkylsulfonyl, (C₄-C₁₀)-cycloalkenylsulfonyl, (C₃-C₁₀)-cycloalkyl-(C₁-C₄)-alkylsulfonyl, (C₄-C₁₀)-cycloalkenyl-(C₁-C₄)-alkylsulfonyl, (C₃-C₁₀)-cycloalkyl-(C₃-C₄)-alkenylsulfonyl, (C₄-C₁₀)-cycloalkenyl-(C₃-C₄)-alkenylsulfonyl, (C₁-C₄)-alkyl-(C₃-C₁₀)-cycloalkylsulfonyl, (C₂-C₄)-alkenyl-(C₃-C₁₀)-cycloalkylsulfonyl, (C₃-C₄)-alkynyl-(C₃-C₁₀)-cycloalkylsulfonyl, (C₁-C₄)-alkyl-(C₄-C₁₀)-cycloalkenylsulfonyl, (C₃-C₄)-alkenyl-(C₄-C₁₀)cycloalkenylsulfonyl, mono- or di-(C₁-C₁₀)-alkylaminosulfonyl or
di-(C₁-C₁₀)-alkylamino, (C₁-C₁₀)-alkylamino, (C₃-C₁₀)-alkenylamino, (C₃-C₁₀)-alkynylamino, (C₃-C₁₀)-cycloalkylamino, (C₄-C₁₀)-cycloalkenylamino, (C₃-C₁₀)-cycloalkyl-(C₁-C₄)-alkylamino, (C₄-C₁₀)-cycloalkenyl-(C₁-C₄)-alkylamino, (C₃-C₁₀)-cycloalkyl-(C₃-C₄)-alkenylamino, (C₄-C₁₀)-cycloalkenyl-(C₃-C₄)-alkenylamino, (C₁-C₄)-alkyl-(C₃-C₁₀)-cycloalkylamino, (C₂-C₄)-alkenyl-(C₃-C₁₀)-cycloalkylamino, (C₂-C₄)-alkynyl-(C₃-C₁₀)-cycloalkylamino, (C₁-C₄)-alkyl-(C₄-C₁₀)-cycloalkenylamino, (C₂-C₄)-alkenyl-(C₄-C₁₀)-cycloalkenylamino or
the N-(C₁-C₄)-alkylamino analogs of the fourteen last-mentioned radicals or
bis-[(C₃-C₁₀)-alkenyl]amino, bis-[(C₃-C₁₀)alkynyl]amino or
tri-[(C₁-C₁₀)-alkyl]silyl or
(C₃-C₁₀)-cycloalkyl, (C₄-C₁₀)-cycloalkenyl, aryl, heterocyclyl, (C₃-C₁₀)-cycloalkylcarbonyl, aroyl, heterocyclylcarbonyl, aryl-(C₁-C₄)-alkylcarbonyl, (C₃-C₁₀)-cycloalkoxycarbonyl, aryloxycarbonyl, heterocyclyloxycarbonyl, aryl-(C₁-C₄)-alkoxycarbonyl, aryloxy, arylthio, arylamino, N-(C₁-C₄)-alkyl-N-arylamino, aryl-(C₁-C₄)-alkoxy, heterocyclyl-(C₁-C₄)-alkoxy, aryl-(C₃-C₄)-alkenyloxy, aryl-(C₁-C₄)-alkylthio, heterocyclyl-(C₁-C₄)-alkylthio, aryl-(C₃-C₄)-alkenylthio, aryl-(C₁-C₄)-alkylamino, N-(C₁-C₄)-alkyl-N-aryl-(C₁-C₄)-alkylamino, aryl-(C₃-C₄)-alkenylamino, N-(C₁-C₄)-alkyl-N-aryl-(C₃-C₄)-alkenylamino, optionally N-substituted arylcarbamoyl or heterocyclylcarbamoyl or heterocyclyl-(C₁-C₄)-alkylcarbamoyl, arylsulfonyl, optionally N-substituted arylsulfonylamino, arylsulfonyl-N-(C₁-C₄)-alkylsulfonyl, optionally N-substituted arylaminosulfonyl or arylaminosulfonylamino, N-aryl-N-(C₁-C₁₀)-alkylaminosulfonyl, heterocyclylsulfonyl, optionally N-substituted heterocyclylsulfonylamino, aryl-di-[(C₁-C₈)-alkyl]silyl, diaryl-(C₁-C₈)-alkylsilyl or triarylsilyl,
   where the cyclic moiety of the 40 last-mentioned radicals is unsubstituted or substituted by one or more radicals from the group consisting of halogen, cyano, nitro, amino, hydroxyl, thio, (C₁-C₆)-alkyl, (C₂-C₆)-alkenyl, (C₂-C₆)-alkynyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl and (C₁-C₄)-alkanoyl.

Further examples for R^{a} and R^{c} are the radicals (C₁-C₁₀)-alkylideneaminooxy, (C₃-C₉)-cycloalkylideneaminooxy (for the formulae —O—N═CR^{O}—R^{a}* and —O—N═CR^{O}—R^{c}* respectively) or 1-[(C₁-C₁₀)-alkoxyimino]-(C₁-C₄)-alkyl, 1-[(C₃-C₉)-cycloalkoxyimino]-(C₁-C₄)-alkyl, 1-hydroxyimino-(C₁-C₄)-alkyl (for the formulae —CR^{O}═N—O—R^{a}* and —CR^{o}═N—O—R^{c}* respectively).

The optionally N-substituted radicals (such as optionally N-substituted arylcarbamoyl, heterocyclylcarbamoyl, arylaminosulfonyl, arylsulfonylamino) are preferably unsubstituted at the amino group or substituted by a radical from the group consisting of (C₁-C₄)-alkyl, (C₁-C₄)-alkanoyl, [(C₁-C₄)-alkoxy]carbonyl and phenyl, in particular unsubstituted or substituted by a radical from the group consisting of (C₁-C₄)-alkyl and phenyl, very particularly unsubstituted or substituted by (C₁-C₄)-alkyl (the latter, for example, N-aryl-N-(C₁-C₄)-alkylcarbamoyl).

The last-mentioned radicals which contain heterocyclyl are preferably those of the formulae
^{N}Het-
^{N}Het-CO
^{N}Het-CO—O—
^{N}Het-CO—NH—
^{N}Het-CO—NR—
^{N}Het-S(O)₂— and
^{N}Het-S(O)₂—NR—,
   where ^{N}Het is the radical of a saturated heterocycle having at least one nitrogen ring atom (N-heterocyclyl) with the free bond (yl position) at the nitrogen ring atom, where ^{N}Het may, additionally to the nitrogen ring atom, contain a further hetero ring atom from the group consisting of N, O and S and this further hetero ring atom is present as a divalent group of the group of the formula —O—, —S—, —SO—, —SO₂—, —NH— or —NR′—, where R and R′ independently of one another are each (C₁-C₄)-alkyl, (C₁-C₄)-alkanoyl, [(C₁-C₄)-alkoxy]carbonyl, di-[(C₁-C₄)-alkyl]carbamoyl or optionally substituted phenyl.

Preferably, R is (C₁-C₄)-alkyl.

Preferably, R′ is (C₁-C₄)-alkyl, (C₁-C₄)-alkanoyl or [(C₁-C₄)-alkoxy]carbonyl.

Preferred substituents R^{b} and R^{c} are those with whom the cycloalkyl, cycloalkenyl, aryl or heterocyclyl groups listed under the radicals R¹ and R² are optionally mono- or polysubstituted (where, in the case of polysubstitution, the substituents can be identical or different), those as defined for R^{a} and R^{b} or else those as listed below:
(C₁-C₁₀)-alkyl, (C₁-C₄)-alkoxy-(C₁-C₄)-alkyl, (C₃-C₁₀)-alkenyl, (C₃-C₁₀)-alkynyl, (C₃-C₁₀)-cycloalkyl-(C₁-C₄)-alkyl, (C₄-C₁₀)-cycloalkenyl-(C₁-C₄)-alkyl, (C₃-C₁₀)-cycloalkyl-(C₃-C₄)-alkenyl, (C₄-C₁₀)-cycloalkenyl-(C₃-C₄)-alkenyl, (C₁-C₄)-alkoxy-(C₃-C₄)-alkenyl,
aryl-(C₁-C₄)-alkyl, heterocyclyl-(C₁-C₄)-alkyl or aryl-(C₃-C₄)-alkenyl,
   where the cyclic moiety of the 3 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl and (C₁-C₄)-alkanoyl,
   preferably, the cyclic moiety of the 3 radicals mentioned is unsubstituted or substituted by one or more radicals from the group consisting of halogen, cyano, nitro, (C₁-C₄)-alkyl, (C₁-C₄)-haloalkyl, (C₁-C₄)-alkoxy and (C₁-C₄)-haloalkoxy.

Adjacent substituents on aryl, heterocyclyl or cycloalkyl groups may optionally, if this is chemically feasible, be attached to form a four- to eight-membered ring.

Particular preference is given to the use according to the invention of compounds (I) in which
R¹ is (C₁-C₆)-alkyl, in particular (C₁-C₄)-alkyl,
   which is unsubstituted or substituted by one or more radicals from the group consisting of hydroxyl, amino, cyano, halogen (in particular fluorine and chlorine), (C₁-C₄)-alkoxy, (C₁-C₄)-alkoxy-(C₁-C₄)-alkoxy, (C₁-C₄)-haloalkoxy (preferably (C₁-C₄)-fluoroalkoxy), (C₃-C₄)-alkenyloxy, (C₃-C₄)-alkynyloxy, (C₁-C₄)-alkylthio, (C₁-C₄)-haloalkylthio (preferably (C₁-C₄)-fluoroalkylthio), (C₁-C₄)-alkenylthio, (C₁-C₄)-alkynylthio, (C₁-C₄)-alkylsulfinyl, (C₁-C₄)-haloalkylsulfinyl (preferably (C₁-C₄)-fluoroalkylsulfinyl), (C₁-C₄)-alkylsulfonyl, (C₁-C₄)-haloalkylsulfonyl (preferably (C₁-C₄)-fluoroalkylsulfonyl), (C₁-C₄)-alkylamino, di-[(C₁-C₄)-alkyl]amino,
   carboxyl, (C₁-C₄)-alkoxycarbonyl, (C₃-C₈)-cycloalkoxycarbonyl, (C₁-C₄)-alkanoyl, (C₁-C₄)-haloalkanoyl, (C₃-C₈)-cycloalkanoyl, carbamoyl, mono- and di-[(C₁-C₄)-alkyl]carbamoyl,
   (C₁-C₄)-alkylsulfonylamino, (C₁-C₄)-alkanoylamino, mono- and di-[(C₁-C₄)-alkyl]aminocarbonylamino, (C₁-C₄)-alkoxycarbonylamino
   and the N-(C₁-C₄)-alkyl analogs of the 5 radicals mentioned above, (C₁-C₄)-alkanoyloxy, (C₁-C₄)-haloalkanoyloxy, (C₃-C₈)-cycloalkanoyloxy, (C₁-C₄)-alkoxycarbonyloxy, (C₁-C₄)-alkylaminocarbonyloxy, di-[(C₁-C₄)-alkyl]-aminocarbonyloxy, (C₃-C₆)-cycloalkyl, heterocyclylcarbonyl, aryl and heteroaryl,
      where each of the 4 last-mentioned radicals is optionally substituted, preferably unsubstituted or substituted by one or more radicals from the group consisting of halogen, (C₁-C₄)-alkyl, (C₁-C₄)-haloalkyl and (C₁-C₄)-alkoxy,
   and a five- to eight-membered, preferably five- or six-membered, saturated heterocycle which has preferably 1 to 3 hetero ring atoms from the group consisting of N, O and S, in particular O and S,
      which is optionally substituted, preferably unsubstituted or substituted by one or more radicals from the group consisting of (C₁-C₄)-alkyl and (C₁-C₄)-alkoxy, preferably (C₁-C₄)-alkyl.

Heterocyclylcarbonyl is preferably a radical of the formula ^{N}Het-CO, where ^{N}Het- is as defined above or as preferably defined.

Particular preference is also given to the use according to the invention of compounds (I) in which
R¹ is (C₃-C₆)-alkenyl,
   which is unsubstituted or substituted by one or more radicals from the group consisting of halogen, preferably fluorine or chlorine, and aryl which is unsubstituted or substituted by one or more radicals from the group consisting of halogen, (C₁-C₄)-alkyl, (C₁-C₄)-haloalkyl and (C₁-C₄)-alkoxy.

Particular preference is also given to the use according to the invention of compounds (I), in which R¹ is (C₃-C₆)-alkynyl or (C₃-C₆)-haloalkynyl.

Particular preference is also given to the use according to the invention of compounds (I) in which R¹ is (C₃-C₆)-cycloalkyl or a five- to eight-membered saturated heterocycle
which is unsubstituted or substituted by one or more radicals from the group consisting of (C₁-C₄)-alkyl and (C₁-C₄)-alkoxy, preferably (C₁-C₄)-alkyl.

Particular preference is also given to the use according to the invention of compounds (I) in which
R² is (C₁-C₈)-alkyl, (C₃-C₈)-cycloalkyl, aryl or heteroaryl, where each of the two last-mentioned radicals is unsubstituted or substituted by one or more radicals from the group consisting of halogen, (C₁-C₄)-alkyl, (C₁-C₄)-haloalkyl and (C₁-C₄)-alkoxy where heteroaryl has preferably 5 or 6 ring atoms, 1 to 3, in particular 1 or 2, of which are hetero ring atoms from the group consisting of N, O and S, very particularly thienyl, furyl, thiazolyl or pyridyl, for example 2-thienyl, 3-thienyl, 2-furyl, 3-furyl, 1,3-thiazol-2-yl, 2-pyridyl, 3-pyridyl or 4-pyridyl.

Some of the compounds of the formula (I) are known or can be prepared analogously to known processes. Their use as safeners in plants has hitherto not been disclosed.

Some compounds of the formula (I) according to the invention or salts thereof are novel and also form part of the subject-matter of the invention.

Accordingly, the invention also provides novel compounds of the formula (I) and salts thereof in which
X is oxygen or sulfur;
(Y)ₙ
   are n substituents Y, where each Y independently of the others is a halogen, cyano, nitro, (C₁-C₆)-alkyl, (C₂-C₆)-alkenyl, (C₂-C₆)-alkynyl, (C₁-C₆)-alkoxy, (C₁-C₆)-alkylthio, (C₁-C₆)-alkylsulfinyl, (C₁-C₆)-alkylsulfonyl, (C₁-C₆)-alkoxycarbonyl, (C₁-C₄)-alkylamino or di-[(C₁-C₄)-alkyl]amino radical, where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
   (C₃-C₆)-cycloalkyl, (C₄-C₆)-cycloalkenyl, aryl or heterocyclyl, where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkoxy-(C₁-C₄)-alkoxy and (C₁-C₄)-alkylthio, or
   two adjacent groups Y together with the carbon atoms which are directly attached are a four- to eight-membered fused-on ring which is carbocyclic or heterocyclic, has one to three hetero ring atoms from the group consisting of N, O and S and is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio,
n is 0, 1, 2, 3 or 4, preferably 0, 1, 2 or 3, in particular 0, 1 or 2, and
R¹
   is (C₁-C₄)-alkyl, (C₃-C₁₀)-alkenyl or (C₃-C₁₀)-alkynyl, where each of the two (2) last-mentioned radicals is unsubstituted or each of the three (3) last-mentioned radicals is substituted by one or more identical or different radicals R^{a} and, including substituents, has 1 to 30 carbon atoms, preferably 1 to 24 carbon atoms, or
   (C₃-C₁₀)-cycloalkyl, (C₄-C₁₀)-cycloalkenyl or saturated heterocyclyl, where each of the 3 last-mentioned radicals is unsubstituted or substituted by one or more identical or different radicals R^{b} and, including substituents, has 3 to 30 carbon atoms, preferably 3 to 24 carbon atoms, and
R²
   is aryl or heterocyclyl,
      where each of the 2 last-mentioned radicals is unsubstituted or substituted by one or more identical or different radicals R^{d} and, including substituents, has 3 to 30 carbon atoms, preferably 3 to 24 carbon atoms,
   where in the radicals R¹ and R² the substituent
   R^{a} is in each case independently of the others a radical from the group consisting of halogen, cyano, nitro and radicals of the formulae -Z^{a}-R^{a}* and R^{cyc-a},
   R^{b} is in each case independently of the others a radical from the group consisting of halogen, cyano, nitro and radicals of the formulae -Z^{b}-R^{b}* and R^{b}**,
   R^{d} is in each case independently of the others a radical from the group consisting of halogen, cyano, nitro and radicals of the formulae -Z^{d}-R^{d}* and R^{d}**,
   where in the radicals R^{a} and R^{b}
   Z^{a} and Z^{b} are each independently of one another a divalent group of the formula
      —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —S—CO—, —CO—S—, —S—CS—, —CS—S—, —O—CO—O—, —NR^{O}—, —O—NR^{O}—, —NR^{O}—O—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— and —SiR′R″—, where in each case p is the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms (and in this case preferably acyl from the group consisting of [(C₁-C₆)-alkyl]carbonyl, [(C₁-C₆)-alkoxy]carbonyl or [(C₁-C₆)-alkylsulfonyl) and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
   R^{cyc-a} is an optionally substituted cyclic hydrocarbon radical having a total of 1 to 24 carbon atoms, preferably 1 to 18 carbon atoms, or an optionally substituted heterocyclic radical having a total of 1 to 24 carbon atoms, preferably 1 to 18 carbon atoms, and
   R^{a}*, R^{b}* and R^{b}** are each independently of one another an optionally substituted hydrocarbon radical having a total of 1 to 24 carbon atoms, preferably 1 to 18 carbon atoms, or an optionally substituted heterocyclic radical having a total of 1 to 24 carbon atoms, preferably 1 to 18 carbon atoms or
   R^{a}* and R^{b}* are each independently of one another hydrogen (preferably if chemically stable radicals are included), and
   where in the radical R^{d}
   Z^{d} is a divalent group of the formula —O—, —S(O)ₚ—, —S(O)ₚ—O—, —O—S(O)ₚ—, —S(O)ₚNR^{O}—, —CO—, —O—CO—, —CO—O—, —S—CO—, —CO—S—, —S—CS—, —CS—S—, —O—CO—O—, —CO—NR^{O}—, —O—CO—NR^{O}— or —SiR′R″— in which p is in each case the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms (preferably acyl from the group consisting of [(C₁-C₆)-alkyl]carbonyl, [(C₁-C₆)-alkoxy]carbonyl and [(C₁-C₆)-alkyl]sulfonyl) and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
   R^{d}* and R^{d}** are each independently of one another an optionally substituted hydrocarbon radical having a total of 1 to 24 carbon atoms or an optionally substituted heterocyclic radical having a total of 1 to 24 carbon atoms or
   R^{d}* is hydrogen,
      except for compounds of the formula (I) and salts thereof in which
(a) R¹ is (C₁-C₄)-alkyl which is substituted by a cyclohexylcarbamoyl radical, and R² is a bicyclic heteroaryl radical,
(b) R¹ is (C₁-C₄)-alkyl which is substituted by a N-substituted carbamoyl radical and at the same time by optionally substituted cycloalkyl, heteroaryl or phenyl, and R² is phenyl,
(c) R¹ is (C₁-C₄)-alkyl which is substituted by 2-(trimethylsilyl)ethoxy, and R² is optionally substituted phenyl,
(d) R² is optionally substituted phenyl or heteroaryl, where one substituent contains more than one cyclic group or where two or more substituents are cyclic,
(e) R¹ is (C₁-C₄)-alkyl, which is substituted, and R² is phenyl which is substituted by iminocarbamoyl (amidine group),
(f) R¹ is (C₁-C₄)-alkyl which is substituted by an optionally substituted aryl radical, and R² is an optionally substituted aryl radical,
(g) R² is an optionally substituted indolyl radical or a N-(4-bromophenyl)- or N-phenyl-5-(hydroxymethyl)pyrazol-3-yl radical and
   also except for the following compounds:
(h) 1-(2-hydroxyethyl)-3-phenylquinoxalin-2 (1H)-one,
(i) 1-[2-(diethylamino)ethyl]-3-phenylquinoxalin-2(1H)-one,
(j) 1-[3-(diethylamino)propyl]-3-phenylquinoxalin-2(1H)-one,
(k) 7-chloro-1-[3-(dimethylamino)propyl]-3-phenylquinoxalin-2 (1H)-one,
(l) 1-{3-[2-(pyrrolidinyl-1-carbonyl)pyrrolidinyl-1-carbonyl]propyl}-3-phenylquinoxalin-2(1H)-one,
(m) 1-{2-[2-(pyrrolidinyl-1-carbonyl)pyrrolidinyl-1-carbonyl]ethyl}-3-phenylquinoxalin-2(1H)-one,
(n) 1-{2-[4-(pyrrolidinyl-1-carbonyl)thiazolidinyl-3-carbonyl]ethyl}-3-phenylquinoxalin-2(1H)-one,
(o) 1-{2-[4-(thiazolidinyl-1-carbonyl)thiazolidinyl-3-carbonyl]ethyl}-3-phenylquinoxalin-2(1H)-one,
(p) 1-{2-[4-(pyrrolidinyl-1-carbonyl)-1,1-dioxothiazolidinyl-3-carbonyl]ethyl}-3-phenylquinoxalin-2(1H)-one,
(q) 1-[3-(amino)propyl]-3-phenylquinoxalin-2 (1H)-one,
(r) 1-(octahydro-2H-quinolizin-1-ylmethyl)-3-phenylquinoxalin-2 (1H)-one,
(s) 6-methoxy- or 6-methyl- or 6-trifluoromethyl- or 6-chloro-1-(octahydro-2H-quinolizin-1-ylmethyl)-3-phenylquinoxalin-2 (1H)-one (4 compounds),
(t) 1-(methylthiomethyl)-3-phenylquinoxalin-2(1H)-one,
(u) 1-(methylaminocarbonylmethyl)-3-(2-ethoxyphenyl)quinoxalin-2 (1H)-one,
(v) 1-(dimethylaminomethyl)-3-(4-ethoxycarbonylphenyl)-6-bromoquinoxalin-2(1H)-one,
(w) 1-(morpholin-4-ylmethyl)-3-(4-ethoxycarbonylphenyl)-6-bromoquinoxalin-2(1H)-one,
(x) 1-(4-benzylpiperid-1-ylmethyl)-3-(4-ethylphenyl)quinoxalin-2 (1H)-one,
(y) 1-(4-benzylpiperazin-1-ylmethyl)-3-(3-chlorophenyl)quinoxalin-2 (1H)-one,
(z) 1-[3-[4-(4,5-dihydropyridazin-3 (2H)-on-6-yl)phenoxy]propyl]-3-phenylquinoxalin-2(1H)-one.

Some of the excluded compounds of the definitions (a) to (z) are known and described in:
Tetrahedron Letters 43 (2002), 1637-1639 (for definitions (a) and (b)),
WO-A-2002/002550 (for definitions (c) and (h)),
Molecular Crystals and Liquid Crystals 329 (1999), 1137-1143 (inter alia definition (d)),
Carbohydrate Research 228 (2003), 2301-2309 (inter alia definition (g))
WO-A-99/50254 (for definitions (e), (j), (k)),
Helv. Chim. Acta XXXV (1952) 2301 (for definitions (h), (i)),
WO-A-97/07116 (for definitions (l), (m), (n), (O), (p)),
Yakugaku Zasshi 90 (1970), 1391-5 (for definition (q)),
II Farmaco 44 (1989), 945-50, 11 Farmaco 41 (1986), 722-8 (for definition (r)),
II Farmaco 40 (1985), 303-314 (for definition (s)),
CAS Registry No. 385798-86-7 (for definition (t))
CAS Registry No. 383408-90-0 (for definition (u))
CAS Registry No. 376619-52-2 (for definition (v))
CAS Registry No. 376616-71-6 (for definition (w))
CAS Registry No. 376605-64-0 (for definition (x))
CAS Registry No. 376604-67-0 (for definition (y))
CAS Registry No. 117826-30-9 from JP-A-63145272 (for definition (z))

Of particular interest are the novel compounds (I) in which the general radicals in formula (I) have the meanings as mentioned above for preferred definitions, where the provisos for the novel compounds as illustrated above have to be taken into account.

Preference is also given to compounds (I) according to the invention in which
R¹
   is (C₁-C₄)-alkyl, (C₃-C₆)-alkenyl or (C₃-C₆)-alkynyl,
      where each of the 2 last-mentioned radicals is unsubstituted or each of the 3 last-mentioned radicals is substituted by one or more identical or different radicals R^{a} and, including substituents, has 1 to 24 carbon atoms, preferably 1 to 18 carbon atoms, or
   (C₃-C₆)-cycloalkyl or saturated heterocyclyl,
      where each of the 2 last-mentioned radicals is unsubstituted or substituted by one or more identical or different radicals R^{b} and, including substituents, has 3 to 24 carbon atoms, preferably 3 to 18 carbon atoms,
   where
   R^{a} in each case independently is a radical from the group consisting of halogen, cyano, nitro and radicals of the formulae -Z^{a}-R^{a}* and R^{cyc-a},
   R^{b} in each case independently is a radical from the group consisting of halogen, cyano, nitro and radicals of the formulae -Z^{b}-R^{b}* and R^{b**},
   where in the radicals R^{a} and R^{b} the radicals or groups
      Z^{a}, Z^{b}, R^{cyc-a}, R^{a}*, R^{b}* and R^{b}** are as defined above or as defined further below,
   preferably
   Z^{a} and Z^{b} independently of one another are —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— or —SiR′R″—, where p is in each case the integer 0, 1 or 2 and the radicals R^{O} are each independently of one another hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl, in particular each hydrogen or (C₁-C₄)-alkyl, and R′ and R″ independently of one another are (C₁-C₄)-alkyl, phenyl, phenyl-(C₁-C₄)-alkyl or (C₃-C₆)-cycloalkyl, in particular (C₁-C₄)-alkyl,
   R^{cyc-a} is (C₃-C₆)-cycloalkyl, (C₄-C₆)-cycloalkenyl, phenyl, saturated heterocyclyl, unsaturated non-aromatic heterocyclyl or heteroaryl, where each of the 6 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of saturated or unsaturated non-aromatic heterocyclyl, also oxo, and
   in particular
   R^{cyc-a} is (C₃-C₆)-cycloalkyl, phenyl, saturated heterocyclyl having 3 to 6 ring atoms or heteroaryl having 5 or 6 ring atoms, where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio and, in the case of saturated heterocyclyl, also oxo, and
   R^{a}*, R^{b}* and R^{b}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, (C₁-C₄)-alkylcarbamoylamino, di-[(C₁-C₄)-alkyl]-carbamoylamino, trimethylsilyl, (C₁-C₄)-alkanoyl, [(C₁-C₄)-alkoxy]-carbonyl, carbamoyl, (C₁-C₄)-alkylcarbamoyl, di-[(C₁-C₄)-alkyl]carbamoyl and, in the case of cyclic radicals, also (C₁-C₄)-alkyl, (C₁-C₄)-haloalkyl, (C₁-C₄)-alkoxy-(C₁-C₄)-alkyl and, in the case of heterocyclyl, also oxo, or
   R^{a}* and R^{b}* are each independently of one another hydrogen,
   in particular
   R^{a}*, R^{b}* and R^{b}** are each independently of one another (C₁-C₆)-alkyl, (C₃-C₆)-cycloalkyl, phenyl, saturated heterocyclyl or heteroaryl, where each of the 5 last-mentioned radicals is unsubstituted or substituted by one or more radicals from the group consisting of halogen, cyano, nitro, (C₁-C₄)-alkoxy, (C₁-C₄)-haloalkoxy, (C₁-C₄)-alkylthio and, in the case of cyclic radicals, also (C₁-C₄)-alkyl, (C₁-C₄)-haloalkyl, (C₁-C₄)-alkoxy-(C₁-C₄)-alkyl and, in the case of saturated heterocyclyl, also oxo, or
   R^{a}* and R^{b}* are each independently of one another hydrogen,
      except for the above-mentioned compounds of the provisos (a) to (z).

Particular preference is given to compounds (I) in which
R^{cyc-a} is (C₃-C₆)-cycloalkyl which is unsubstituted or mono- or polysubstituted by (C₁-C₄)-alkyl, or phenyl or saturated heterocyclyl having 3 to 6 ring atoms or heteroaryl having 5 or 6 ring atoms, where each of the 3 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio and, in the case of saturated heterocyclyl, also oxo.

Particular preference is given to compounds (I) in which
R^{a}*, R^{b}* and R^{b}** are each independently of one another
   (C₁-C₄)-alkyl, (C₃-C₆)-cycloalkyl, phenyl, saturated heterocyclyl having 3 to 6 ring atoms or heteroaryl having 5 or 6 ring atoms, where each of the 5 last-mentioned radicals is unsubstituted or substituted by one or more radicals from the group consisting of halogen, (C₁-C₄)-alkoxy, (C₁-C₄)-haloalkoxy, (C₁-C₄)-alkylthio and, in the case of cyclic radicals, also (C₁-C₄)-alkyl, (C₁-C₄)-haloalkyl, (C₁-C₄)-alkoxy-(C₁-C₄)-alkyl and, in the case of saturated heterocyclyl, also oxo, or
   R^{a}* and R^{b}* are each independently of one another hydrogen.

More preference is also given to compounds (I) according to the invention in which
R²
   is phenyl or heteroaryl,
      where each of the 2 last-mentioned radicals is unsubstituted or substituted by one or more identical or different radicals R and, including substituents, has 3 to 24 carbon atoms, preferably 3 to 18 carbon atoms,
   where R^{d} are each independently an inorganic or organic radical, preferably a radical from the group consisting of halogen, cyano, nitro and radicals of the formulae -Z^{d}-R^{d}* and R^{d}**,
   where in the radicals R^{d} the radicals or groups
      Z^{d}, R^{d}* and R^{d}** are as defined above or further below,
   preferably
   Z^{d} are each independently of one another a divalent group of the formula —O—, —S(O)ₚ—, —S(O)ₚ—O—, —O—S(O)ₚ—, —S(O)ₚNR^{O}—, —CO—, —O—CO—, —CO—O—, —CO—NR^{O}—, —O—CO—NR^{O}— or —SiR′R″—, where each p is the integer 0, 1 or 2 and the radicals R^{O} are each independently of one another hydrogen, (C₁-C₄)-alkyl, phenyl, phenyl-(C₁-C₄)-alkyl, (C₃-C₆)-cycloalkyl, (C₁-C₄)— alkanoyl, [(C₁-C₄)-alkoxy]carbonyl or (C₁-C₄)-alkylsulfonyl, in particular each hydrogen or (C₁-C₄)-alkyl, and R′ and R″ are independently of one another (C₁-C₄)-alkyl, phenyl, phenyl-(C₁-C₄)-alkyl or (C₃-C₆)-cycloalkyl, in particular (C₁-C₄)-alkyl,
   R^{d}* and R^{d}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, (C₁-C₄)-alkylcarbamoylamino, di-[(C₁-C₄)-alkyl]carbamoylamino, trimethylsilyl, (C₁-C₄)-alkanoyl, [(C₁-C₄)-alkoxy]carbonyl, carbamoyl, (C₁-C₄)-alkylcarbamoyl, di-[(C₁-C₄)-alkyl]carbamoyl and, in the case of cyclic radicals, also (C₁-C₄)-alkyl, (C₁-C₄)-haloalkyl, (C₁-C₄)-alkoxy-(C₁-C₄)alkyl and, in the case of heterocyclyl, also oxo, or
   R^{d} are each independently of one another hydrogen,
   in particular
      R^{d}* and R^{d}** are each independently of one another (C₁-C₆)-alkyl, (C₃-C₆)-cycloalkyl, phenyl, saturated heterocyclyl or heteroaryl, where each of the 5 last-mentioned radicals is unsubstituted or substituted by one or more radicals from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio and, in the case of saturated heterocyclyl, also oxo, or
   R^{d} are each independently of one another hydrogen,
      except for the above-mentioned compounds of the provisos (a) to (z).

Particular preference is given to compounds (I) in which
Z^{d} are each independently of one another a divalent group of the formula —O—, —S(O)ₚ—, —CO—, —O—CO—, —CO—O—, —CO—NR^{O}— or —O—CO—NR^{O}—,
   where p is the integer 0, 1 or 2 and the radicals R^{O} are each independently of one another hydrogen or (C₁-C₄)-alkyl.

Particular preference is given to compounds (I) in which
R^{d}* and R^{d}** are each independently of one another
   (C₁-C₄)-alkyl, (C₃-C₆)-cycloalkyl, phenyl, saturated heterocyclyl having 3 to 6 ring atoms or heteroaryl having 5 or 6 ring atoms, where each of the 5 last-mentioned radicals is unsubstituted or substituted by one or more radicals from the group consisting of halogen, (C₁-C₄)-alkoxy, (C₁-C₄)-haloalkoxy, (C₁-C₄)-alkylthio and, in the case of cyclic radicals, also (C₁-C₄)-alkyl, (C₁-C₄)-haloalkyl, (C₁-C₄)-alkoxy-(C₁-C₄)-alkyl and, in the case of saturated heterocyclyl, also oxo, or
   R^{a}* and R^{b}* are each independently of one another hydrogen.

Examples of substituents R^{a} and R^{c} or R^{d} with whom the alkyl, alkenyl and alkynyl groups or the cyclic radicals listed under radicals R¹ and R² are optionally mono- or polysubstituted (where in the case of polysubstitution the substituents can be identical or different) are the suitable exemplary compounds which have already been mentioned above for the use according to the invention.

Preferably
R¹ is mono- or polysubstituted (C₁-C₄)-alkyl, possible substituents at the alkyl group being:
halogen, cyano, amino or
(C₁-C₄)-alkoxy, (C₁-C₄)-haloalkoxy, preferably (C₁-C₄)-fluoroalkoxy, (C₁-C₄)-alkoxy-(C₁-C₄)-alkoxy, (C₃-C₄)-alkenyloxy, (C₃-C₄)-alkynyloxy or
carbamoyl, mono- or di-[(C₁-C₄)-alkyl]carbamoyl, mono- or di-[(C₃-C₁₀)-cycloalkyl]-carbamoyl, N-(C₁-C₄)-alkoxy-N-(C₁-C₄)-alkylcarbamoyl or
carboxyl, (C₁-C₁₀)-alkoxycarbonyl, (C₃-C₁₀)-cycloalkoxycarbonyl, (C₁-C₁₀)-alkanoyloxy, (C₄-C₁₀)-cycloalkanoyloxy, (C₁-C₁₀)-alkoxycarbonyloxy, [(C₁-C₁₀)-alkyl]aminocarbonyloxy, di-[(C₁-C₁₀)-alkyl]aminocarbonyloxy, or
(C₁-C₁₀)-alkylsulfonylamino, (C₁-C₁₀)-alkanoylamino, (C₃-C₁₀)-alkenoylamino, (C₄-C₁₀)-cycloalkanoylamino, (C₃-C₁₀)-cycloalkyl-(C₁-C₄)-alkanoylamino, mono- or di-[(C₁-C₁₀)-alkyl]aminocarbonylamino, or
[(C₁-C₁₀)-alkoxy]carbonylamino or
the N-(C₁-C₄)-alkyl analogs of the 8 radicals mentioned above or
(C₁-C₄)-alkylthio, (C₁-C₄)-haloalkylthio, preferably (C₁-C₄)-fluoroalkylthio, (C₃-C₄)— alkenylthio, (C₃-C₄)-alkynylthio, or
(C₁-C₁₀)-alkylsulfinyl, (C₁-C₄)-haloalkylsulfinyl, (C₃-C₁₀)-alkenylsulfinyl, (C₃-C₁₀)-alkynylsulfinyl, (C₃-C₁₀)-cycloalkylsulfinyl, (C₄-C₁₀)-cycloalkenylsulfinyl, (C₃-C₁₀)-cycloalkyl-(C₁-C₄)-alkylsulfinyl, (C₄-C₁₀)-cycloalkenyl-(C₁-C₄)-alkylsulfinyl, (C₃-C₁₀)-cycloalkyl-(C₃-C₄)-alkenylsulfinyl, (C₄-C₁₀)-cycloalkenyl-(C₃-C₄)-alkenylsulfinyl, (C₁-C₄)-alkyl-(C₃-C₁₀)-cycloalkylsulfinyl, (C₂-C₄)-alkenyl-(C₃-C₁₀)-cycloalkylsulfinyl, (C₂-C₄)-alkynyl-(C₃-C₁₀)-cycloalkylsulfinyl, (C₁-C₄)-alkyl-(C₄-C₁₀)-cycloalkenylsulfinyl, (C₂-C₄)-alkenyl-(C₄-C₁₀)-cycloalkenylsulfinyl, (C₂-C₄)-alkynyl-(C₄-C₁₀)-cycloalkenylsulfinyl or
(C₁-C₁₀)-alkylsulfonyl, (C₁-C₄)-haloalkylsulfinyl, (C₃-C₁₀)-alkenylsulfonyl, (C₃-C₁₀)-alkynylsulfonyl, (C₃-C₁₀)-cycloalkylsulfonyl, (C₄-C₁₀)-cycloalkenylsulfonyl, (C₃-C₁₀)-cycloalkyl-(C₁-C₄)-alkylsulfonyl, (C₄-C₁₀)-cycloalkenyl-(C₁-C₄)-alkylsulfonyl, (C₃-C₁₀)-cycloalkyl-(C₃-C₄)-alkenylsulfonyl, (C₄-C₁₀)-cycloalkenyl-(C₃-C₄)-alkenylsulfonyl, (C₁-C₄)-alkyl-(C₃-C₁₀)-cycloalkylsulfonyl, (C₂-C₄)-alkenyl-(C₃-C₁₀)-cycloalkylsulfonyl, (C₃-C₄)-alkynyl-(C₃-C₁₀)-cycloalkylsulfonyl, (C₁-C₄)-alkyl-(C₄-C₁₀)-cycloalkenylsulfonyl, (C₃-C₄)-alkenyl-(C₄-C₁₀)-cycloalkenylsulfonyl, mono- or di-(C₁-C₁₀)-alkylaminosulfonyl or
di-(C₁-C₁₀)-alkylamino, (C₁-C₁₀)-alkylamino, (C₃-C₁₀)-alkenylamino, (C₃-C₁₀)-alkynylamino, (C₃-C₁₀)-cycloalkylamino, (C₄-C₁₀)-cycloalkenylamino, (C₃-C₁₀)-cycloalkyl-(C₁-C₄)-alkylamino, (C₄-C₁₀)-cycloalkenyl-(C₁-C₄)-alkylamino, (C₃-C₁₀)-cycloalkyl-(C₃-C₄)-alkenylamino, (C₄-C₁₀)-cycloalkenyl-(C₃-C₄)-alkenylamino, (C₁-C₄)-alkyl-(C₃-C₁₀)-cycloalkylamino, (C₂-C₄)-alkenyl-(C₃-C₁₀)-cycloalkylamino, (C₂-C₄)-alkynyl-(C₃-C₁₀)-cycloalkylamino, (C₁-C₄)-alkyl-(C₄-C₁₀)-cycloalkenylamino, (C₂-C₄)-alkenyl-(C₄-C₁₀)-cycloalkenylamino or
the N-(C₁-C₄)-alkylamino analogs of the fourteen last-mentioned radicals or
bis-[(C₃-C₁₀)-alkenyl]amino, bis-[(C₃-C₁₀)-alkynyl]amino or
tri-[(C₁-C₁₀)-alkyl]silyl or
(C₃-C₁₀)-cycloalkyl, heterocyclyl, (C₃-C₁₀)-cycloalkylcarbonyl, benzoyl, heterocyclylcarbonyl, phenyl-(C₁-C₄)-alkylcarbonyl, (C₃-C₁₀)-cycloalkoxycarbonyl, phenoxycarbonyl, heterocyclyloxycarbonyl, phenyl-(C₁-C₄)-alkoxycarbonyl, phenoxy, phenylthio, phenylamino, N-(C₁-C₄)-alkyl-N-phenylamino, phenyl-(C₁-C₄)-alkoxy, heterocyclyl-(C₁-C₄)-alkoxy, phenyl-(C₃-C₄)-alkenyloxy, phenyl-(C₁-C₄)-alkylthio, heterocyclyl-(C₁-C₄)-alkylthio, phenyl-(C₃-C₄)-alkenylthio, phenyl-(C₁-C₄)-alkylamino, N-(C₁-C₄)-alkyl-N-phenyl-(C₁-C₄)-alkylamino, phenyl-(C₃-C₄)-alkenylamino, N-(C₁-C₄)-alkyl-N-phenyl-(C₃-C₄)-alkenylamino, optionally N-substituted phenylcarbamoyl or heterocyclylcarbamoyl or heterocyclyl-(C₁-C₄)-alkylcarbamoyl, phenylsulfonyl, optionally N-substituted phenylsulfonylamino, phenylsulfonyl-N-(C₁-C₄)-alkylsulfonyl, optionally N-substituted phenylaminosulfonyl or phenylaminosulfonylamino, N-phenyl-N-(C₁-C₁₀)-alkylaminosulfonyl, heterocyclylsulfonyl, optionally N-substituted heterocyclylsulfonylamino, phenyl-di-[(C₁-C₈)-alkyl]silyl, diphenyl-(C₁-C₈)-alkylsilyl or triphenylsilyl,
   where the cyclic moiety of the 39 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-haloalkylthio, (C₁-C₄)-alkylamino and di-[(C₁-C₄)-alkyl]amino.

The optionally N-substituted radicals (such as optionally N-substituted phenylcarbamoyl, heterocyclylcarbamoyl, phenylaminosulfonyl, phenylsulfonylamino) are preferably unsubstituted at the amino group or substituted by a radical from the group consisting of (C₁-C₄)-alkyl, (C₁-C₄)-alkanoyl, [(C₁-C₄)-alkoxy]carbonyl and phenyl, in particular unsubstituted or substituted by a radical from the group consisting of (C₁-C₄)-alkyl and phenyl, very particularly unsubstituted or substituted by (C₁-C₄)-alkyl (the latter for example N-phenyl-N-(C₁-C₄)-alkylcarbamoyl).

The last-mentioned radicals which contain heterocyclyl are preferably those of the formulae
^{N}Het-
^{N}Het-CO
^{N}Het-CO—O—
^{N}Het-CO—NH—
^{N}Het-CO—NR—
^{N}Het-S(O)₂— and
^{N}Het-S(O)₂—NR—,
   where ^{N}Het is the radical of a saturated heterocycle having at least one nitrogen ring atom (N-heterocyclyl) with the free bond (yl position) at the nitrogen ring atom, where ^{N}Het may, additionally to the nitrogen ring atom, contain a further hetero ring atom from the group consisting of N, O and S and this further hetero ring atom is present as a divalent group of the group of the formula —O—, —S—, —SO—, —SO₂—, —NH— or —NR′—, where R and R′ independently of one another are each (C₁-C₄)-alkyl, (C₁-C₄)— alkanoyl, [(C₁-C₄)-alkoxy]carbonyl, di-[(C₁-C₄)-alkyl]carbamoyl or optionally substituted phenyl.

Preferably, R is (C₁-C₄)-alkyl.

Preferably, R′ is (C₁-C₄)-alkyl, (C₁-C₄)-alkanoyl or [(C₁-C₄)-alkoxy]carbonyl.

Preference is given to compounds (I) in which
R² is heteroaryl or aryl which has optionally up to three identical or different substituents, these substituents being (C₁-C₄)-alkyl, (C₁-C₄)-haloalkyl, halogen or alkoxy and heteroaryl preferably being thienyl, furyl, thiazolyl or pyridyl, in particular thienyl or pyridyl.

Examples of compounds (I) to be used according to the invention are listed in the tables further below.

The compounds of the formula (I) can be prepared, for example, by
(a) reacting a compound of the formula (II) 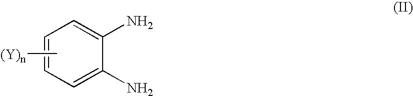
   in which (Y)ₙ is as defined in formula (I) with an α-keto acid derivative of the formula (III) 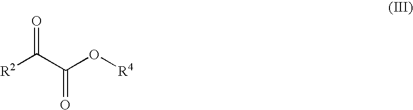
   in which R² is as defined in formula (I) and R⁴ is hydrogen, optionally substituted alkyl or optionally substituted aryl to give a compound of the formula (Ia) 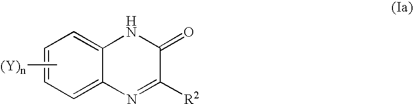
   in which (Y)ₙ and R² are as defined in formula (I),
   and converting this compound of the formula (Ia) by reaction with an alkylating agent of the formula (IV)
      R¹-L (IV)
   in which R¹ is as defined in formula (I) and L is a leaving group, such as, for example, chlorine, bromine, iodine, optionally substituted alkylsulfonyl (preferably (C₁-C₄)-alkylsulfonyl, such as, for example, methylsulfonyl or ethylsulfonyl) or optionally substituted arylsulfonyl (preferably optionally substituted arylsulfonyl, such as, for example, phenylsulfonyl or p-toluenesulfonyl), or,
   in the specific case where R¹ is a methyl group, using the alkylating agent dimethylformamide dimethyl acetal,
   into the compound of the formula (I) or a salt thereof,
(b) reacting a compound of the formula (V) 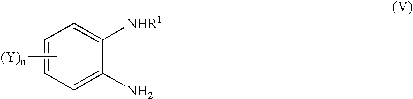
   in which R¹ and (Y)ₙ are as defined in formula (I)
   with an α-keto acid derivative of the formula (II) mentioned under (a) or
(c) derivatizing a compound of the formula (I′) 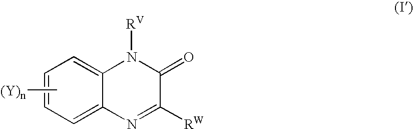
   in which (Y)ₙ is as defined in formula (I),
   the radical R^{v} is different from R¹ but a precursor of R¹ and the radical R^{w} is identical to R² or
   the radical R^{w} is different from R² but a precursor of R² and the radical R^{v} is identical to R¹,
   at the radical referred to as “precursor” by known or customary methods using one or more process steps, to give the compound of the formula (I).

The cyclizations to give the quinoxalinones according to variants (a) and (b) can be carried out, for example, in water or in an inert organic solvent, in a temperature range between 20° C. and 150° C., preferably between 50° C. and 100° C. Suitable organic solvents are, for example, polar protic or aprotic solvents, such as ethers, for example diethyl ether, tetrahydrofuran and dioxane, or nitriles, such as acetonitrile, or amides, such as dimethylformamide, or alcohols, such as methanol or ethanol.

The reaction of the compounds (Ia) with the alkylating agent of the formula (IV) to give the product of the formula (I) is preferably carried out in an inert organic solvent in the presence of an acid-binding agent and in a temperature range between 20° C. and 150° C., preferably between 50° C. and 100° C. Suitable organic solvents are, for example, polar protic or aprotic solvents, such as ethers, for example tetrahydrofuran, dioxane and dioxolane, or nitriles, such as acetonitrile, or amides, such as dimethylformamide, or sulfoxides, such as dimethyl sulfoxide, or ketones, such as acetone, or alcohols, such as methanol or ethanol. Acid-binding agents are, for example, alkali metal or alkaline earth metal carbonates, such as, for example, sodium carbonate, potassium carbonate or calcium carbonate, alkali metal or alkaline earth metal hydroxides, such as sodium hydroxide, potassium hydroxide or calcium hydroxide, or alkali metal hydrides or amides, such as sodium hydride or sodium amide or potassium hydride or potassium amide, or else organic bases, such as triethylamine, pyridine, dimethylaminopyridine, DBU (1,8-diazabicyclo[5.4.0]undec-7-ene), DBN (1,5-diazabicyclo[4.3.0]non-5-ene) and 1,4-diazabicyclo[2.2.2]octane.

In the case of dimethylformamide dimethyl acetal, the product of the formula (I) can be prepared by reacting the reaction partners neat or in an inert organic solvent at elevated temperature, expediently in a range between 80° C. and 150° C.

Suitable derivatization reactions for the process (c) are, starting with compounds of the formula (I′) which can be prepared analogously to processes (a) and (b) and which are already compounds of the formula (I) or similar compounds having different functional groups, a large number of reactions which are customary or known to the person skilled in the art. Here, the precursors are derivatized to give the radicals R¹ and/or R² in question.

Moreover, the carbonyl group in the compound (I′) can be derivatized to give the thione group (to X=S in formula (I)), for example by sulfurization using P₂S₅ or Lawesson's reagent (cf. March's Advanced Organic Chemistry, Wiley 2001, p. 1184).

The compounds of the formulae (II), (III), (IV) and (V) are either commercially available or can be prepared by or analogously to methods known to the person skilled in the art (for example J. Heterocyclic Chem 31 (1994) 775; Helv. Chim. Acta 35 (1952) 2301; DE 1078131; Tetrahedron 53 (1997) 16767).

The invention also provides a method for protecting crop plants or useful plants against phytotoxic actions of agrochemicals, such as pesticides or, in particular, herbicides which cause damage to plants, which method comprises using compounds of the formula (I) or salts thereof as safeners, preferably by applying an effective amount of the compounds of the formula (I) or their salts to the plants, to parts of plants or their seeds (or seed).

The safeners, together with active compounds (pesticides), are suitable for the selective control of harmful organisms in a number of plant crops, for example in crops of economic importance, such as cereals (wheat, barley, triticale, rye, rice, corn, millet), sugar beet, sugar cane, oilseed rape, cotton and soybeans. Of particular interest is the use in monocotyledonous crops, such as cereals (wheat, barley, rye, triticale, sorghum), including corn and rice, and monocotyledonous vegetable crops, but also in dicotyledonous crops, such as, for example, soybean, oilseed rape, cotton, grape vines, vegetable plants, fruit plants and ornamental plants. Also of interest are mutant crops which are completely or partially tolerant to some pesticides or transgenic crops which are completely or partially tolerant, for example corn crops resistant to glufosinate or glyphosate, or soybean crops resistant to herbicidal imidazolinones. However, the particular advantage of the novel use of the safeners is their effective action in crops which are normally not sufficiently tolerant to the pesticides mentioned.

For the joint use with pesticides, the compounds of the formula (I) according to the invention can be applied simultaneously with the active compounds or in any order, and they are then capable of reducing or completely eliminating harmful side effects of these active compounds in crop plants, without negatively affecting or substantially reducing the activity of these active compounds against unwanted harmful organisms. Here, even damage caused by using a plurality of pesticides, for example a plurality of herbicides or herbicides in combination with insecticides or fungicides, can be reduced substantially or eliminated completely. In this manner, it is possible to extend the field of use of conventional pesticides considerably.

If the compositions according to the invention comprise pesticides, these compositions are, after appropriate dilution, applied either directly to the area under cultivation, to the already germinated harmful and/or useful plants or to the already emerged harmful and/or useful plants. If the compositions according to the invention do not comprise any pesticide, these compositions can be employed by the tank mix method—i.e. the user mixes and dilutes the separately available products (=the pesticide and the agent protecting the useful plants) immediately prior to application to the area to be treated—or prior to the application of a pesticide, or after the application of a pesticide, or for the pretreatment of seed, i.e., for example, for dressing the seed of the useful plants.

The advantageous actions of the compounds (I) according to the invention are observed when they are used together with the pesticides by the pre-emergence method or the post-emergence method, for example in the case of simultaneous application as a tank mix or a coformulation or in the case of a separate application, in parallel or in succession (split application). It is also possible to repeat the application a number of times. In some cases, it may be expedient to combine a pre-emergence application with a post-emergence application. In most cases, one option is a post-emergence application to the useful plant or crop plant together with a simultaneous or later application of the pesticide. Also possible is the use of the compounds (I) according to the invention for seed dressing, for (dip) treatment of seedlings (for example rice) or for the treatment of other propagation material (for example potato tubers).

When using the compounds (I) according to the invention in combination with herbicides, in addition to the safener action, enhanced action, e.g. herbicidal action, against harmful plants is frequently also observed. Furthermore, in many cases, there is an improved growth of the useful plants and crop plants, and it is possible to increase the harvest yields.

Some of the last-mentioned advantageous actions are also observed when the compounds (I) are used without additional pesticides, in particular when other environmental factors negatively affect plant growth.

The compositions according to the invention may comprise one or more pesticides. Suitable pesticides are, for example, herbicides, insecticides, fungicides, acaricides and nematicides, which, when used on their own, would cause phytotoxic damage to the crop plants or would probably cause damage. Of particular interest are corresponding pesticidally active compounds from the groups of the herbicides, insecticides, acaricides, nematicides and fungicides, in particular herbicides.

The weight ratio of safener to pesticide can be varied within wide limits and is generally in the range from 1:100 to 100:1, preferably from 1:20 to 20:1, in particular from 1:10 to 10:1. The optimum weight ratio of safener to pesticide depends both on the respective safener used and the respective pesticide, and on the type of useful plant or crop plant to be protected. The required application rate of safener can, depending on the pesticide used and the type of useful plant to be protected, be varied within wide limits and is generally in the range from 0.001 to 10 kg, preferably from 0.005 to 5 kg, in particular from 0.1 to 1 kg, of safener per hectare. The weight ratios and amounts required for a successful treatment can be determined by simple preliminary experiments.

For seed dressing, for example, from 0.005 to 20 g of safener per kilogram of seed, preferably from 0.01 to 10 g of safener per kilogram of seed, in particular from 0.05 to 5 g of safener per kilogram of seed, are used.

If solutions of safener are used for seed treatment and the seeds or seedlings are wetted with the solutions, the suitable concentration is generally in the range from 1 to 10 000 ppm, preferably from 100 to 1000 ppm, based on the weight. The weight ratios and amounts required for a successful treatment can be determined by simple preliminary experiments.

The safeners can be formulated in the customary manner, separately or together with the pesticides. Accordingly, the present invention also provides the useful-plant-protecting or crop-plant-protecting compositions.

Preferred is the joint application of safener and pesticide, in particular that of safener and herbicide as a readymix or the use by the tankmix method.

Insecticides which may cause damage to plants when used on their own or together with herbicides are, for example, the following:
Organophosphates, for example terbufos (Counter®), fonofos (Dyfonate®), phorate (Thimet®), chlorpyriphos (Reldan®), carbamates, such as carbofuran (Furadan®), pyrethroid insecticides, such as tefluthrin (Force®), deltamethrin (Decis®) and tralomethrin (Scout®), and other insecticidal agents having a different mechanism of action.

Herbicides whose phytotoxic side effects on crop plants can be reduced using compounds of the formula (I) can be from entirely different structural classes and have entirely different mechanisms of action. Preference is given to commercially available herbicides as described, for example, in the handbook “The Pesticide Manual”, 13th Edition 2003, The British Crop Protection Council, and the e-Pesticide Manual Version 3 (2003), or else in the “Compendium of Pesticide Common Names” (searchable via the Internet) and in literature quoted therein. The herbicides and plant growth regulators mentioned hereinbelow by way of example are in each case referred to by their standardized common active compound name according to the “International Organization for Standardization” (ISO), or by the chemical name or the code number. Examples of active compounds whose phytotoxic action in crop plants and useful plants can be reduced by the compounds (I) according to the invention are: acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; alachlor; alloxydim(-sodium); ametryn; amicarbazone, amidochlor, amidosulfuron; aminopyralid, amitrol; AMS, i.e. ammonium sulfamate; anilofos; asulam; atrazine; azafenidin; azimsulfuron (DPX-A8947); aziprotryn; barban; BAS 516H, i.e. 5-fluoro-2-phenyl-4H-3,1-benzoxazin-4-one; beflubutamid; benazolin(-ethyl); benfluralin; benfuresate; bensulfuron(-methyl); bensulide; bentazone(-sodium); benzfendizone, benzobicyclone; benzofenap; benzofluor; benzoylprop(-ethyl); benzthiazuron; bialaphos (bilanafos); bifenox; bispyribac-(-sodium); bromacil; bromobutide; bromofenoxim; bromoxynil; bromuron; buminafos; busoxinone; butachlor; butafenacil; butamifos; butenachlor; buthidazole; butralin; butroxydim; butylate; cafenstrole (CH-900); carbetamide; carfentrazone(-ethyl); caloxydim, CDAA, i.e. 2-chloro-N,N-di-2-propenylacetamide; CDEC, i.e. 2-chloroallyl diethyldithiocarbamate; chlomethoxyfen; chloramben; chlorazifop-butyl; chlorbromuron; chlorbufam; chlorfenac; chlorfenprop, chlorflurenol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron, cinidon-(-methyl or -ethyl), cinmethylin; cinosulfuron; clethodim; clefoxydim, clodinafop and its ester derivatives (for example clodinafop-propargyl); clomazone; clomeprop; cloprop, cloproxydim; clopyralid; clopyrasulfuron(-methyl); cloransulam(-methyl); cumyluron (JC 940); cyanazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-D; 2,4-DB; dalapon; dazomet, desmedipham; desmetryn; di-allate; dicamba; dichlobenil; dichlorprop(-P); diclofop and its esters such as diclofop-methyl; diclosulam, diethatyl(-ethyl); difenoxuron; difenzoquat; diflufenican; diflufenzopyr(-sodium); dimefuron; dimepiperate; dimethachlor; dimethametryn; dimethenamid (SAN-582H); dimethenamid(-P); dimethazone, dimethipin; dimexyflam, dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan, EPTC; esprocarb; ethalfluralin; ethametsulfuron-methyl; ethidimuron; ethiozin; ethofumesate; ethoxyfen and its esters (for example ethyl ester, HC-252), ethoxysulfuron, etobenzanid (HW 52); F5231, i.e. N-[2-chloro-4-fluoro-5-[4-(3-fluoropropyl)-4,5-dihydro-5-oxo-1H-tetrazol-1-yl]-phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide; fenuron; flamprop(-methyl or -isopropyl or -isopropyl-L); flazasulfuron; florasulam; fluazifop and fluazifop-P and their esters, for example fluazifop-butyl and fluazifop-P-butyl; fluazolate, flucarbazone(-sodium); flucetosulfuron, fluchloralin; fldfenacet (FOE 5043), flufenpyr(-ethyl), flumetsulam; flumeturon; flumiclorac-(-pentyl); flumioxazin (S-482); flumipropyn; fluometuron; fluorochloridone, fluorodifen; fluoroglycofen(-ethyl); flupoxam (KNW-739); flupropacil (UBIC-4243); fluproanate, flupyrsulfuron(-methyl, or -sodium); flurenol(-butyl); fluridone; flurochloridone; fluroxypyr(-meptyl); flurprimidol, flurtamone; fluthiacet(-methyl); fluthiamide (also known as flufenacet); fomesafen; foramsulfuron; fosamine; furilazole (MON 13900), furyloxyfen; glufosinate (-ammonium); glyphosate(-isopropylammonium); halosafen; halosulfuron(-methyl) and its esters (for example the methyl ester, NC-319); haloxyfop and its esters; haloxyfop-P (=R-haloxyfop) and its esters; HC-252 (diphenyl ether), hexazinone; imazamethabenz(-methyl); imazamethapyr; imazamox; imazapic, imazapyr; imazaquin and salts such as the ammonium salts; imazethamethapyr; imazethapyr, imazosulfuron; indanofan; iodosulfuron-(methyl)-(sodium), ioxynil; isocarbamid; isopropalin; isoproturon; isouron; isoxaben; isoxachlortole; isoxaflutole; isoxapyrifop; karbutilate; lactofen; lenacil; linuron; MCPA; MCPA-thioethyl, MCPB; mecoprop(-P); mefenacet; mefluidid; mesosulfuron(-methyl); mesotrione; metam, metamifop, metamitron; metazachlor; methabenzthiazuron; methazole; methoxyphenone; methyldymron; metobenzuron, metobromuron; (S-)metolachlor; metosulam (XRD 511); metoxuron; metribuzin; metsulfuron-methyl; MK-616; molinate; monalide; monocarbamide dihydrogensulfate; monolinuron; monuron; MT 128, i.e. 6-chloro-N-(3-chloro-2-propenyl)-5-methyl-N-phenyl-3-pyridazinamine; MT 5950, i.e. N-[3-chloro-4-(1-methylethyl)-phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; othosulfamuron; oryzalin; oxadiargyl (RP-020630); oxadiazone; oxasulfuron; oxaziclomefone; oxyfluorfen; paraquat; pebulate; pelargonic acid; pendimethalin; penoxulam; pentanochlor, pentoxazone; perfluidone; pethoxamid, phenisopham; phenmedipham; picloram; picolinafen; pinoxaden; piperophos; piributicarb; pirifenopbutyl; pretilachlor; primisulfuron(-methyl); procarbazone(-sodium); procyazine; prodiamine; profluazole, profluralin; profoxydim; proglinazine(-ethyl); prometon; prometryn; propachlor; propanil; propaquizafop; propazine; propham; propisochlor; propoxycarbazone(-sodium), propyzamide; prosulfalin; prosulfocarb; prosulfuron (CGA-152005); prynachlor; pyraclonil, pyraflufen(-ethyl); pyrazolinate; pyrazon; pyrazosulfuron(-ethyl); pyrazoxyfen; pyribenzoxim; pyributicarb; pyridafol; pyridate; pyriftalid, pyrimidobac(-methyl); pyrimisulfan; pyrithiobac(-sodium) (KIH-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and its methyl ester; sulcotrione; sulfentrazone (FMC-97285, F-6285); sulfazuron; sulfometuron(-methyl); sulfosate (ICI-A0224); sulfosulfuron; TCA; tebutam (GCP-5544); tebuthiuron; tepraloxydim; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchior (NSK-850); thiafluamide; thiazafluron; thiazopyr (Mon-13200); thidiazimin (SN-24085); thidiazuron, thifensulfuron(-methyl); thiobencarb; tiocarbazil; topramezone; tralkoxydim; tri-allate; triasulfuron; triaziflam; triazofenamide; tribenuron(-methyl); 2,3,6-trichlorobenzoic acid (2,3,6-TBA), triclopyr; tridiphane; trietazine; trifloxysulfuron(-sodium), trifluralin; triflusulfuron and esters (e.g. methyl ester, DPX-66037); trimeturon; tritosulfuron; tsitodef; vernolate; WL 110547, i.e. 5-phenoxy-1-[3-(trifluoromethyl)phenyl]-1H-tetrazole; UBH-509; D-489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127; KIH-2023 and KIH5996.

Herbicides, whose phytotoxic side effects on crop plants can be reduced using compounds of the formula (I) are, for example, herbicides from the group of the carbamates, thiocarbamates, haloacetanilides, substituted phenoxy-, naphthoxy- and phenoxyphenoxycarboxylic acid derivatives and heteroaryloxyphenoxyalkanecarboxylic acid derivatives, such as quinolyloxy-, quinoxalyloxy-, pyridyloxy-, benzoxazolyloxy- and benzothiazolyloxyphenoxyalkanecarboxylic acid esters, cyclohexanedione oximes, benzoylcyclohexanediones, benzoylisoxazoles, benzoylpyrazoles, imidazolinones, pyrimidinyloxypyridinecarboxylic acid derivatives, pyrimidyloxybenzoic acid derivatives, sulfonylureas, sulfonylaminocarbonyltriazolinones, triazolopyrimidinesulfonamide derivatives, phosphinic acid derivatives and salts thereof, glycine derivatives, triazolinones, triazinones and also S-(N-aryl-N-alkylcarbamoylmethyl)dithiophosphoric esters, pyridinecarboxylic acids, pyridines, pyridinecarboxamides, 1,3,5-triazines and others.

Preference is given to phenoxyphenoxy- and heteroaryloxyphenoxycarboxylic acid esters and salts, cyclohexanedione oximes, benzoylcyclohexanediones, benzoylisoxazoles, benzoylpyrazoles, sulfonylureas, sulfonylaminocarbonyltriazolinones, imidazolinones and mixtures of the active compounds mentioned with one another and/or with active compounds used for broadening the activity spectrum of the herbicides, for example bentazone, cyanazine, atrazine, bromoxynil, dicamba and other leaf-acting herbicides.

Herbicides which are suitable for combination with the safeners according to the invention are, for example:
A) herbicides of the type of the phenoxyphenoxy- and heteroaryloxyphenoxycarboxylic acid derivatives, such as
A1) phenoxyphenoxy- and benzyloxyphenoxycarboxylic acid derivatives, for example methyl 2-(4-(2,4-dichlorophenoxy)phenoxy)propionate (diclofop-methyl),
methyl 2-(4-(4-bromo-2-chlorophenoxy)phenoxy)propionate (DE-A 26 01 548),
methyl 2-(4-(4-bromo-2-fluorophenoxy)phenoxy)propionate (U.S. Pat. No. 4,808,750),
methyl 2-(4-(2-chloro-4-trifluoromethylphenoxy)phenoxy)propionate (DE-A 24 33 067),
methyl 2-(4-(2-fluoro-4-trifluoromethylphenoxy)phenoxy)propionate (U.S. Pat. No. 4,808,750),
methyl 2-(4-(2,4-dichlorobenzyl)phenoxy)propionate (DE-A 24 17 487),
ethyl 4-(4-(4-trifluoromethylphenoxy)phenoxy)pent-2-enoate,
methyl 2-(4-(4-trifluoromethylphenoxy)phenoxy)propionate (DE-A 24 33 067),
butyl (R)-2-[4-(4-cyano-2-fluorophenoxy)phenoxy]propionate (cyhalofop-butyl)
A2) “monocyclic” heteroaryloxyphenoxyalkanecarboxylic acid derivatives, for example
ethyl 2-(4-(3,5-dichloropyridyl-2-oxy)phenoxy)propionate (EP-A 0 002 925),
propargyl 2-(4-(3,5-dichloropyridyl-2-oxy)phenoxy)propionate (EP-A 0 003 114),
methyl (RS)- or (R)-2-(4-(3-chloro-5-trifluoromethyl-2-pyridyloxy)phenoxy)propionate (haloxyfop-methyl or haloxyfop-P-methyl),
ethyl 2-(4-(3-chloro-5-trifluoromethyl-2-pyridyloxy)phenoxy)propionate (EP-A 0 003 890),
propargyl 2-(4-(5-chloro-3-fluoro-2-pyridyloxy)phenoxy)propionate (clodinafop-propargyl),
butyl (RS)- or (R)-2-(4-(5-trifluoromethyl-2-pyridyloxy)phenoxy)propionate (fluazifop-butyl or fluazifop-P-butyl),
(R)-2-[4-(3-chloro-5-trifluoromethyl-2-pyridyloxy)phenoxy]propionic acid
A3) “bicyclic” heteroaryloxyphenoxyalkanecarboxylic acid derivatives, for example
methyl and ethyl (RS)- or (R)-2-(4-(6-chloro-2-quinoxalyloxy)phenoxy)propionate (quizalofop-methyl and -ethyl or quizalofop-P-methyl and -P-ethyl),
methyl 2-(4-(6-fluoro-2-quinoxalyloxy)phenoxy)propionate (see J. Pest. Sci. Vol. 10, 61 (1985)),
2-isopropylidenaminooxyethyl (R)-2-(4-(6-chloro-2-quinoxalyloxy)phenoxy)propionate (propaquizafop),
ethyl (RS)- or (R)-2-(4-(6-chlorobenzoxazol-2-yloxy)phenoxy)propionate (fenoxaprop-ethyl or fenoxaprop-P-ethyl),
ethyl 2-(4-(6-chlorobenzthiazol-2-yloxy)phenoxy)propionate (DE-A-26 40 730),
tetrahydro-2-furylmethyl (RS)- or (R)-2-(4-(6-chloroquinoxalyloxy)phenoxy)propionate (EP-A-0 323 727);
B) herbicides from the group of the sulfonylureas, such as pyrimidinyl- or triazinylaminocarbonyl[benzene-, -pyridine-, -pyrazole-, -thiophene- and -(alkylsulfonyl)alkylamino]sulfamides. Preferred substituents on the pyrimidine ring or the triazine ring are alkoxy, alkyl, haloalkoxy, haloalkyl, halogen or dimethylamino, it being possible to combine all substituents independently of one another. Preferred substituents in the benzene, pyridine, pyrazole, thiophene or (alkylsulfonyl)alkylamino moiety are alkyl, alkoxy, halogen, nitro, alkoxycarbonyl, aminocarbonyl, alkylaminocarbonyl, dialkylaminocarbonyl, alkoxyaminocarbonyl, haloalkoxy, haloalkyl, alkylcarbonyl, alkoxyalkyl, (alkanesulfonyl)alkylamino. Such suitable sulfonylureas are, for example,
B1) phenyl- and benzylsulfonylureas and related compounds, for example
1-(2-chlorophenylsulfonyl)-3-(4-methoxy-6-methyl-1,3,5-triazin-2-yl)urea (chlorsulfuron),
1-(2-ethoxycarbonylphenylsulfonyl)-3-(4-chloro-6-methoxypyrimidin-2-yl)urea (chlorimuron-ethyl),
1-(2-methoxyphenylsulfonyl)-3-(4-methoxy-6-methyl-1,3,5-triazin-2-yl)urea (metsulfuron-methyl),
1-(2-chloroethoxyphenylsulfonyl)-3-(4-methoxy-6-methyl-1,3,5-triazin-2-yl)urea (triasulfuron),
1-(2-methoxycarbonylphenylsulfonyl)-3-(4,6-dimethylpyrimidin-2-yl)urea (sulfumeturon-methyl),
1-(2-methoxycarbonylphenylsulfonyl)-3-(4-methoxy-6-methyl-1,3,5-triazin-2-yl)-3-methylurea (tribenuron-methyl),
1-(2-methoxycarbonylbenzylsulfonyl)-3-(4,6-dimethoxypyrimidin-2-yl)urea (bensulfuron-methyl),
1-(2-methoxycarbonylphenylsulfonyl)-3-(4,6-bis-(difluoromethoxy)pyrimidin-2-yl)urea (primisulfuron-methyl),
3-(4-ethyl-6-methoxy-1,3,5-triazin-2-yl)-1-(2,3-dihydro-1,1-dioxo-2-methylbenzo[b]-thiophene-7-sulfonyl)urea (EP-A 0 079 683),
3-(4-ethoxy-6-ethyl-1,3,5-triazin-2-yl)-1-(2,3-dihydro-1,1-dioxo-2-methylbenzo[b]-thiophene-7-sulfonyl)urea (EP-A 0 079 683),
3-(4-methoxy-6-methyl-1,3,5-triazin-2-yl)-1-(2-methoxycarbonyl-5-iodophenylsulfonyl)urea (WO 92/13845),
methyl 2-[4-dimethylamino-6-(2,2,2-trifluoroethoxy)-1,3,5-triazin-2-ylcarbamoylsulfamoyl]-3-methylbenzoate (DPX-66037, triflusulfuron-methyl),
oxetan-3-yl 2-[(4,6-dimethylpyrimidin-2-yl)carbamoylsulfamoyl]benzoate (CGA-277476, oxasulfuron),
methyl 4-iodo-2-[3-(4-methoxy-6-methyl-1,3,5-triazin-2-yl)ureidosulfonyl]benzoate, sodium salt (iodosulfuron-methyl-sodium),
methyl 2-[3-(4,6-dimethoxypyrimidin-2-yl)ureidosulfonyl]-4-methanesulfonylaminomethylbenzoate (mesosulfuron-methyl, WO 95/10507),
N,N-dimethyl-2-[3-(4,6-dimethoxypyrimidin-2-yl)ureidosulfonyl]-4-formylaminobenzamide (foramsulfuron, WO 95/01344),
1-(4,6-dimethoxy-1,3,5-triazin-2-yl)-3-[2-(2-methoxyethoxy)phenylsulfonyl]urea (cinosulfuron),
methyl 2-[(4-ethoxy-6-methylamino-1,3,5-triazin-2-yl)carbamoylsulfamoyl]benzoate (ethametsulfuron-methyl),
1-(4-methoxy-6-methyl-1,3,5-triazin-2-yl)-3-[2-(3,3,3-trifluoropropyl)phenylsulfonyl]-urea (prosulfuron),
methyl 2-(4,6-dimethylpyrimidin-2-ylcarbamoylsulfamoyl)benzoate (sulfometuron-methyl),
1-(4-methoxy-6-trifluoromethyl-1,3,5-triazin-2-yl)-3-(2-trifluoromethylbenzenesulfonyl)urea (tritosulfuron);
B2) thienylsulfonylureas, for example
1-(2-methoxycarbonylthiophen-3-yl)-3-(4-methoxy-6-methyl-1,3,5-triazin-2-yl)urea (thifensulfuron-methyl);
B3) pyrazolylsulfonylureas, for example
1-(4-ethoxycarbonyl-1-methylpyrazol-5-ylsulfonyl)-3-(4,6-dimethoxypyrimidin-2-yl)-urea (pyrazosulfuron-ethyl),
methyl 3-chloro-5-(4,6-dimethoxypyrimidin-2-ylcarbamoylsulfamoyl)-1-methylpyrazole-4-carboxylate (halosulfuron-methyl),
methyl 5-(4,6-dimethylpyrimidin-2-yl-carbamoylsulfamoyl)-1-(2-pyridyl)pyrazole-4-carboxylate (NC-330, see Brighton Crop Prot. Conference ‘Weeds’ 1991, Vol. 1, p. 45 ff.),
1-(4,6-dimethoxypyrimidin-2-yl)-3-[1-methyl-4-(2-methyl-2H-tetrazol-5-yl)pyrazol-5-yl-sulfonyl]urea (DPX-A8947, azimsulfuron);
B4) sulfonediamide derivatives, for example
3-(4,6-dimethoxypyrimidin-2-yl)-1-(N-methyl-N-methylsulfonylaminosulfonyl)urea (amidosulfuron) and its structural analogs (EP-A 0 131 258 and Z. Pfl. Krankh. Pfl. Schutz, special issue XII, 489-497 (1990));
B5) pyridylsulfonylureas, for example
1-(3-N,N-dimethylaminocarbonylpyridin-2-ylsulfnyl)-3-(4,6-dimethoxypyrimidin-2-yl)-urea (nicosulfuron),
1-(3-ethylsulfonylpyridin-2-ylsufonyl)-3-(4,6-dimethoxypyrimidin-2-yl)urea (rimsulfuron),
methyl 2-[3-(4,6-dimethoxypyrimidin-2-yl)ureidosulfonyl]-6-trifluoromethyl-3-pyridinecarboxylate, sodium salt (DPX-KE 459, flupyrsulfuron-methyl-sodium),
3-(4,6-dimethoxypyrimidin-2-yl)-1-(3-N-methylsulfonyl-N-methylaminopyridin-2-yl)-sulfonylurea or its salts (DE-A 40 00 503 and DE-A 40 30 577), 1-(4,6-dimethoxypyrimidin-2-yl)-3-(3-trifluoromethyl-2-pyridylsulfonyl)urea (flazasulfuron),
1-(4,6-dimethoxypyrimidin-2-yl)-3-[3-(2,2,2-trifluoroethoxy)-2-pyridylsulfonyl]urea sodium salt (trifloxysulfuron-sodium);
B6) alkoxyphenoxysulfonylureas, for example
3-(4,6-dimethoxypyrimidin-2-yl)-1-(2-ethoxyphenoxy)sulfonylurea or its salts (ethoxysulfuron);
B7) imidazolylsulfonylureas, for example
1-(4,6-dimethoxypyrimidin-2-yl)-3-(2-ethylsulfonylimidazo[1,2-a]pyridin-3-yl)sulfonylurea (MON 37500, sulfosulfuron),
1-(2-chloroimidazo[1,2-a]pyridin-3-ylsulfonyl)-3-(4,6-dimethoxypyrimidin-2-yl)urea (imazosulfuron);
B8) phenylaminosulfonylureas, for example
1-[2-(cyclopropylcarbonyl)phenylaminosulfonyl]-3-(4,6-dimethoxypyrimidin-2-yl)urea (cyclosulfamuron);
C) chloroacetanilides, for example
acetochlor, alachlor, butachlor, dimethachlor, dimethenamid, metazachlor, metolachlor, S-metolachlor, pethoxamid, pretilachlor, propachlor, propisochlor and thenylchlor;
D) thiocarbamates, for example
S-ethyl N,N-dipropylthiocarbamate (EPTC),
S-ethyl N,N-diisobutylthiocarbamate (butylate); cycloate, dimepiperate, esprocarb, molinate, orbencarb, pebulate, prosulfocarb, thiobencarb, tiocarbazil and tri-allate;
E) cyclohexanedione oximes, for example
alloxydim, butroxydim, clethodim, cloproxydim, cycloxydim, protoxydim, sethoxydim, tepraloxydim and tralkoxydim;
F) imidazolinones, for example
imazamethabenz-methyl, imazapic, imazamox, imazapyr, imazaquin and imazethapyr;
G) triazolopyrimidinesulfonamide derivatives, for example chloransulam-methyl, diclosulam, florasulam, flumetsulam, metosulam and penoxulam;
H) benzoylcyclohexanediones, for example
2-(2-chloro-4-methylsulfonylbenzoyl)cyclohexane-1,3-dione (SC-0051, sulcotrione),
2-(2-nitrobenzoyl)-4,4-dimethylcyclohexane-1,3-dione (EP-A 0 274 634),
2-(2-nitro-3-methylsulfonylbenzoyl)-4,4-dimethylcyclohexane-1,3-dione (WO 91/13548),
2-[4-(methylsulfonyl)-2-nitrobenzoyl]-1,3-cyclohexanedione (mesotrione);
2-[2-chloro-3-(5-cyanomethyl-4,5-dihydroisoxazol-3-yl)-4-(ethylsulfonyl)benzoyl]-1,3-cyclohexanedione,
2-[2-chloro-3-(5-cyanomethyl-4,5-dihydroisoxazol-3-yl)-4-(methylsulfonyl)benzoyl]-1,3-cyclohexanedione,
2-[2-chloro-3-(5-ethoxymethyl-4,5-dihydroisoxazol-3-yl)-4-(ethylsulfonyl)benzoyl]-1,3-cyclohexanedione,
2-[2-chloro-3-(5-ethoxymethyl-4,5-dihydroisoxazol-3-yl)-4-(methylsulfonyl)benzoyl]-1,3-cyclohexanedione,
2-[2-chloro-3-[(2,2,2-trifluoroethoxy)methyl]-4-(ethylsulfonyl)benzoyl]-1,3-cyclohexanedione,
2-[2-chloro-3-[(2,2,2-trifluoroethoxy)methyl]-4-(methylsulfonyl)benzoyl]-1,3-cyclohexanedione,
2-[2-chloro-3-[(2,2-difluoroethoxy)methyl]-4-(ethylsulfonyl)benzoyl]-1,3-cyclohexanedione,
2-[2-chloro-3-[(2,2-difluoroethoxy)methyl]-4-(methylsulfonyl)benzoyl]-1,3-cyclohexanedione,
2-[2-chloro-3-[(2,2,3,3-tetrafluoropropoxy)methyl]-4-(ethylsulfonyl)benzoyl]-1,3-cyclohexanedione,
2-[2-chloro-3-[(2,2,3,3-tetrafluoropropoxy)methyl]-4-(methylsulfonyl)benzoyl]-1,3-cyclohexanedione,
2-[2-chloro-3-(cyclopropylmethoxy)-4-(ethylsulfonyl)benzoyl]-1,3-cyclohexanedione,
2-[2-chloro-3-(cyclopropylmethoxy)-4-(methylsulfonyl)benzoyl]-1,3-cyclohexanedione,
2-[2-chloro-3-(tetrahydrofuran-2-ylmethoxymethyl)-4-(ethylsulfonyl)benzoyl]-1,3-cyclohexanedione,
2-[2-chloro-3-(tetrahydrofuran-2-ylmethoxymethyl)-4-(methylsulfonyl)benzoyl]-1,3-cyclohexanedione,
2-[2-chloro-3-[2-(2-methoxyethoxy)ethoxymethyl]-4-(ethylsulfonyl)benzoyl]-1,3-cyclohexanedione,
2-[2-chloro-3-[2-(2-methoxyethoxy)ethoxymethyl]-4-(methylsulfonyl)benzoyl]-1,3-cyclohexanedione,
I) benzoylisoxazoles, for example
5-cyclopropyl-[2-(methylsulfonyl)-4-(trifluoromethyl)benzoyl]isoxazole (isoxaflutole);
J) benzoylpyrazoles, for example
2-[4-(2,4-dichloro-m-toluyl)-1,3-dimethylpyrazol-5-yloxy]-4′-methylacetophenone (benzofenap),
4-(2,4-dichlorobenzoyl)-1,3-dimethylpyrazol-5-yl toluene-4-sulfonate (pyrazolynate),
2-[4-(2,4-dichlorobenzoyl)-1,3-dimethylpyrazol-5-yloxy]acetophenone (pyrazoxyfen);
5-hydroxy-1-methyl-4-[2-(methylsulfonyl)-4-trifluoromethylbenzoyl]pyrazole (WO 01/74785),
1-ethyl-5-hydroxy-4-[2-(methylsulfonyl)-4-trifluoromethylbenzoyl]pyrazole (WO 01/74785),
1,3-dimethyl-5-hydroxy-4-[2-(methylsulfonyl)-4-trifluoromethylbenzoyl]pyrazole (WO 01/74785),
1-ethyl-5-hydroxy-3-methyl-4-[2-(methylsulfonyl)-4-trifluoromethylbenzoyl]pyrazole (WO 01/74785),
5-hydroxy-1-methyl-4-[-2-chloro-3-(4,5-dihydroisoxazol-3-yl)-4-methylsulfonylbenzoyl]pyrazole (WO 99/58509),
5-hydroxy-1-methyl-4-[3-(4,5-dihydroisoxazol-3-yl)-2-methyl-4-methylsulfonylbenzoyl]pyrazole (WO 99/58509),
1-ethyl-5-hydroxy-3-methyl-4-[2-methyl-4-methylsulfonyl-3-(2-methoxyethylamino)benzoyl]pyrazole (WO 96/26206),
3-cyclopropyl-5-hydroxy-1-methyl-4-[2-methyl-4-methylsulfonyl-3-(2-methoxyethylamino)benzoyl]pyrazole (WO 96/26206),
5-benzoxy-1-ethyl-4-[2-methyl-4-methylsulfonyl-3-(2-methoxyethylamino)benzoyl]-pyrazole (WO 96/26206),
1-ethyl-5-hydroxy-4-(3-dimethylamino-2-methyl-4-methylsulfonylbenzoyl)pyrazole (WO 96/26206),
5-hydroxy-1-methyl-4-(2-chloro-3-dimethylamino-4-methylsulfonylbenzoyl)pyrazole (WO 96/26206),
1-ethyl-5-hydroxy-4-(3-allylamino-2-chloro-4-methylsulfonylbenzoyl)pyrazole (WO 96/26206),
1-ethyl-5-hydroxy-4-(2-methyl-4-methylsulfonyl-3-morpholinobenzoyl)pyrazole (WO 96/26206),
5-hydroxy-1-isopropyl-4-(2-chloro-4-methylsulfonyl-3-morpholinobenzoyl)pyrazole (WO 96/26206),
3-cyclopropyl-5-hydroxy-1-methyl-4-(2-chloro-4-methylsulfonyl-3-morpholinobenzoyl)pyrazole (WO 96/26206),
1,3-dimethyl-5-hydroxy-4-(2-chloro-4-methylsulfonyl-3-pyrazol-1-ylbenzoyl)pyrazole (WO 96/26206),
1-ethyl-5-hydroxy-3-methyl-4-(2-chloro-4-methylsulfonyl-3-pyrazol-1-ylbenzoyl)-pyrazole (WO 96/26206),
1-ethyl-5-hydroxy-4-(2-chloro-4-methylsulfonyl-3-pyrazol-1-ylbenzoyl)pyrazole (WO 96/26206),
K) sulfonylaminocarbonyltriazolinones, for example
4,5-dihydro-3-methoxy-4-methyl-5-oxo-N-(2-trifluoromethoxyphenylsulfonyl)-1H-1,2,4-triazole-1-carboxamide sodium salt (flucarbazone-sodium), methyl 2-(4,5-dihydro-4-methyl-5-oxo-3-propoxy-1H-1,2,4-triazol-1-yl)carboxamidosulfonylbenzoate sodium salt (propoxycarbazone-Na);
L) triazolinones, for example
4-amino-N-tert-butyl-4,5-dihydro-3-isopropyl-5-oxo-1,2,4-1H-triazole-1-carboxamide (amicarbazone),
2-(2,4-dichloro-5-prop-2-ynyloxyphenyl)-5,6,7,8-tetrahydro-1,2,4-triazolo[4,3-a]-pyridin-3 (2H)-one (azafenidin),
ethyl (RS)-2-chloro-3-[2-chloro-5-(4-difluoromethyl-4,5-dihydro-3-methyl-5-oxo-1H-1,2,4-triazol-1-yl)-4-fluorophenyl]propionate (carfentrazone-ethyl),
2′,4′-dichloro-5′-(4-difluoromethyl-4,5-dihydro-3-methyl-5-oxo-1H-1,2,4-triazol-1-yl)-methanesulfonanilide (sulfentrazone);
M) phosphinic acids and derivatives, for example
4-[hydroxy(methyl)phosphinoyl]-L-homoalanyl-L-alanyl-L-alanine (bilanafos),
DL-homoalanin-4-yl(methyl)phosphinic acid ammonium salt (glufosinate-ammonium);
N) glycine derivatives, for example
N-(phosphonomethyl)glycine and its salts (glyphosate and salts, for example the sodium salt or the isopropylammonium salt),
N-(phosphonomethyl)glycine trimesium salt (sulfosate);

O) pyrimidinyloxypyridinecarboxylic acid derivatives and pyrimidinyloxybenzoic acid derivatives, for example
benzyl 3-(4,6-dimethoxypyrimidin-2-yl)oxypyridine-2-carboxylate (EP-A 0 249 707),
methyl 3-(4,6-dimethoxypyrimidin-2-yl)oxypyridine-2-carboxylate (EP-A 0 249 707),
1-(ethoxycarbonyloxyethyl) 2,6-bis[(4,6-dimethoxypyrimidin-2-yl)oxy]benzoate (EP-A 0 472 113),
2,6-bis[(4,6-dimethoxypyrimidin-2-yl)oxy]benzoic acid (bispyribac-sodium), pyribenzoxim, pyriftalid, pyriminobac-methyl and pyrithiobac-sodium;
P) S-(N-aryl-N-alkylcarbamoylmethyl)dithiophosphonic acid esters, such as S-[N-(4-chlorophenyl)-N-isopropylcarbamoylmethyl]O,O-dimethyl dithiophosphate (anilophos);
Q) triazinones, for example
3-cyclohexyl-6-dimethylamino-1-methyl-1,3,5-triazine-2,4-(1H,3H)-dione (hexazinone),
4-amino-4,5-dihydro-3-methyl-6-phenyl-1,2,4-triazin-5-one (metamitron),
4-amino-6-tert-butyl-4,5-dihydro-3-methylthio-1,2,4-triazin-5-one (metribuzin);
R) pyridinecarboxylic acids, for example
clopyralid, fluroxypyr, picloram and triclopyr;
S) pyridines, for example
dithiopyr and thiazopyr;
T) pyridinecarboxamides, for example
diflufenican and picolinafen;
U) 1,3,5-triazines, for example
ametryn, atrazine, cyanazine, dimethametrin, prometon, prometryn, propazine, simazine, symetryn, terbumeton, terbuthylazine, terbutryn and trietazine;
V) plant growth regulators, for example
forchlorfenuron and thidiazuron.

W) ketoenoles, for example
8-(2,6-diethyl-p-tolyl)-1,2,4,5-tetrahydro-7-oxo-7H-pyrazolo[1,2-d][1,4,5]oxadiazepin-9-yl 2,2-dimethylpropionate (pinoxaden).

The herbicides of groups A to W are known, for example, from the respective abovementioned publications and from “The Pesticide Manual”, The British Crop Protection Council, 13th Edition, 2003, or the e-Pesticide Manual, Version 3.0, British Crop Protection Council 2003.

The compounds of the formula (I) and their combinations with one or more of the abovementioned pesticides can be formulated in various ways, depending on the prevailing physicochemical and biological parameters. Examples of suitable formulation types are:
emulsifiable concentrates which are prepared by dissolving the active compounds in an organic solvent, for example butanol, cyclohexanone, dimethylformamide, xylene or else relatively high-boiling hydrocarbons or mixtures of the organic solvents with addition of one or more ionic and/or nonionic surfactants (emulsifiers). Suitable emulsifiers are, for example, calcium alkylarylsulfonates, fatty acid polyglycol esters, alkylaryl polyglycol ethers, fatty alcohol polyglycol ethers, propylene oxide/ethylene oxide condensates, alkyl polyethers, sorbitan esters and polyoxyethylenesorbitan fatty acid esters;
dusts, which are obtained by grinding the active compounds with finely dispersed inorganic or organic substances, for example talc, natural clays, such as kaolin, bentonite and pyrophyllite, diatomaceous earth or meals;
water- or oil-based suspension concentrates, which can be prepared, for example, by wet grinding using bead mills;
water-soluble powders;
water-soluble concentrates;
granules, such as water-soluble granules, water-dispersible granules and granules for application by broadcasting and soil application;
wettable powders which, in addition to active compound, also contain diluents or inert substances and surfactants;
capsule suspensions and microcapsules;
ultra-low-volume formulations.

The abovementioned formulation types are known to the person skilled in the art and described, for example, in: K. Martens, “Spray Drying Handbook”, 3rd Ed., G. Goodwin Ltd., London, 1979; W. van Valkenburg, “Pesticide Formulations”, Marcel Dekker, N.Y. 1973; Winnaker-Küchler, “Chemische Technologie” [Chemical Technology], volume 7, C. Hanser Verlag Munich, 4th edition 1986; “Perry's Chemical Engineer's Handbook”, 5th Ed., McGraw-Hill, N.Y. 1973, pages 8-57.

The formulation auxiliaries required, such as inert materials, surfactants, solvents and other additives are also known and are described, for example, in: McCutcheon's “Detergents and Emulsifiers Annual”, MC Publ. Corp., Ridgewood N.J.; C. Marsden, “Solvents Guide”, 2nd Ed., Interscience, N.Y. 1963; H. von Olphen, “Introduction to Clay Colloid Chemistry”, 2nd Ed., J. Wiley & Sons, N.Y.; Schönfeldt, “Grenzflächenaktive Äthylenoxidaddukte” [Surface-active ethylene oxide adducts], Wiss. Verlagsgesellschaft, Stuttgart 1976; Sisley and Wood, “Encyclopedia of Surface Active Agents”, Chem. Publ. Co. Inc., N.Y. 1964; Watkins, “Handbook of Insecticide Dust Diluents and Carriers”, 2nd Ed., Darland Books, Caldwell N.J.; Winnacker-Küchler, “Chemische Technologie” [Chemical Technology], volume 7, C. Hanser Verlag Munich, 4th edition 1986.

In addition to the abovementioned formulation auxiliaries, the useful-plant-protecting compositions may comprise, if appropriate, customary tackifiers, wetting agents, dispersants, penetrants, emulsifiers, preservatives, antifreeze agents, fillers, carriers, colorants, anti-foams, evaporation inhibitors and pH or viscosity regulators.

Depending on the formulation type, the useful-plant-protecting compositions generally comprise 0.1 to 99% by weight, in particular 0.2 to 95% by weight, of one or more safeners of the formula (I) or a combination of safener and pesticide. Furthermore, they comprise 1 to 99.9, in particular 4 to 99.5, % by weight of one or more solid or liquid additives and 0 to 25, in particular 0.1 to 25, % by weight of a surfactant. In emulsifiable concentrates, the concentration of active compound, i.e. the concentration of safener and/or pesticide, is generally 1 to 90, in particular 5 to 80, % by weight. Dusts usually comprise 1 to 30, preferably 5 to 20, % by weight of active compound. In wettable powders, the concentration of active compound is generally 10 to 90% by weight. In water-dispersible granules, the content of active compound is, for example, between 1 and 95% by weight, preferably between 10 and 80% by weight.

For use, the formulations, which are present in commercially available form, are, if appropriate, diluted in a customary manner, for example in the case of wettable powders, emulsifiable concentrates, dispersions and water-dispersible granules, with water. Preparations in the form of dusts, granules and sprayable solutions are usually not diluted with any further inert substances prior to use. The required application rate of the safeners varies with the external conditions such as, inter alia, temperature, humidity and the type of herbicide used.

In the examples below, which illustrate the invention but do not limit it, the amounts are based on weight, unless defined otherwise.

### EXAMPLES

1 Formulation Examples

1.1 Dusting Agents

A dusting agent is obtained by mixing 10 parts by weight of a compound of the formula (I) or of an active compound mixture of a pesticide (for example herbicide) and a safener of the formula (I) and 90 parts by weight of talc as inert material and comminuting in a hammer mill.

1.2 Water-Dispersible Powder

A wettable powder which is readily dispersible in water is obtained by mixing 25 parts by weight of a compound of the formula (I) or of an active compound mixture of a pesticide (for example herbicide) and a safener of the formula (I), 64 parts by weight of kaolin-containing quartz as inert material, 10 parts by weight of potassium ligninsulfonate and 1 part by weight of sodium oleoylmethyltaurinate as wetting and dispersing agent, and grinding in a pin mill.

1.3 Water-Dispersible Concentrate

A dispersion concentrate which is readily dispersible in water is obtained by mixing 20 parts by weight of a compound of the formula (I) or of an active compound mixture of a pesticide (for example herbicide) and a safener of the formula (I) with 6 parts by weight of alkylphenol polyglycol ether (®Triton X 207), 3 parts by weight of isotridecanol polyglycol ether and 71 parts by weight of paraffinic mineral oil and grinding in a ball mill to a fineness of below 5 microns.

1.4 Emulsifiable Concentrate

An emulsifiable concentrate is obtained from 15 parts by weight of a compound of the formula (I) or of an active compound mixture of a pesticide (for example herbicide) and a safener of the formula (I), 75 parts by weight of cyclohexanone as solvent and 10 parts by weight of ethoxylated nonylphenol as emulsifier.

1.5 Water-Dispersible Granules

Water-dispersible granules are obtained by mixing
75 parts by weight of a safener of the formula (I) or of a mixture of a pesticide and a safener of the formula (I),
10 ″ of calcium ligninsulfonate,
5 ″ of sodium lauryl sulfate,
3 ″ of polyvinyl alcohol and
7 ″ of kaolin,
   grinding in a pin mill and granulating the powder in a fluidized bed by spraying on water as granulation liquid.

Water-dispersible granules are also obtained by homogenizing
25 parts by weight of a safener of the formula (I) or of a mixture of a pesticide and a safener of the formula (I),
5 ″ of sodium 2,2′-dinaphthylmethane-6,6′-disulfonate,
2 ″ of sodium oleoylmethyltaurinate,
17 ″ of calcium carbonate,
50 ″ of water and
1 part by weight of polyvinyl alcohol
   in a colloid mill, comminuting, then grinding in a bead mill and atomizing and drying the resulting suspension in a spray tower using a single-fluid nozzle.
   2. Preparation Examples

### Example A

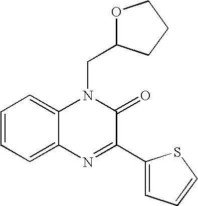

### 1-Tetrahydrofurfuryl-3-(2-thienyl)-1,2-dihydroquinoxalin-2-one

A mixture of 1.00 g (4.4 mmol) of N-tetrahydrofurfuryl-o-phenylenediamine hydrochloride, 0.49 g (4.8 mmol) of triethylamine and 0.81 g (4.4 mol) of ethyl (2-thienyl)glyoxylate in 20 ml of ethanol was heated under reflux for 8 hours. The mixture was concentrated, the residue was taken up in water/dichloromethane and the organic phase was dried and concentrated. For purification, the residue was chromatographed on silica gel (ethyl acetate/heptane 1:1). This gave 0.22 g (16.0% of theory) of a slightly yellowish solid of melting point 123° C.

### Example B

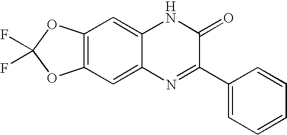

### 6,7-(Difluoromethylenedioxy)-3-phenyl-1,2-dihydroquinoxalin-2-one

A mixture of 1.25 g (4.79 mmol) of 4,5-(difluoromethylenedioxy)-o-phenylenediamine bishydrochloride, 1.07 g (10.53 mmol) of triethylamine and 0.79 g (4.79 mmol) of methyl phenylglyoxylate in 50 ml of methanol was heated under reflux for 8 hours. Even whilst still hot, the product precipitates as a colorless solid. After cooling, the product was filtered off with suction and the filter cake was washed with a little methanol. This gave 1.25 g of product (86.3% of theory) as a colorless solid of melting point 291-292° C.

### Example C

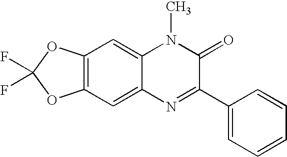

### 6,7-(Difluoromethylenedioxy)-1-methyl-3-phenyl-1,2-dihydroquinoxalin-2-one

0.55 g (1.81 mmol) of 6,7-(difluoromethylenedioxy)-3-phenyl-1,2-dihydroquinoxalin-2-one (example B) and 0.65 g (5.49 mmol) of dimethylformamide dimethyl acetal in 25 ml of dimethylformamide was stirred at 95° C. for 8 hours. After cooling, the mixture was concentrated, the residue was taken up in dilute aqueous sodium hydroxide solution and dichloromethane and the organic phase was washed with water, dried and concentrated. For purification, the residue was chromatographed on silica gel (heptane/ethyl acetate 4:1). This gave 0.32 g of product (55.1% of theory) as a slightly yellowish solid of melting point 165° C.

### Example D

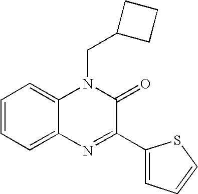

### 1-Cyclobutylmethyl-3-(2-thienyl)-1,2-dihydroquinoxalin-2-one

A mixture of 0.46 g (2 mmol) of 3-(2-thienyl)-1,2-dihydroquinoxalin-2-one (prepared analogously to example B from o-phenylenediamine and ethyl (2-thienyl)glyoxylate), 0.30 g (2 mmol) of bromomethylcyclobutane and 0.28 g (2 mmol) of potassium carbonate in 10 ml of dimethylformamide was stirred at 90° C. for 5 hours. After cooling, the mixture was concentrated and the residue was taken up in water/dichloromethane. The organic phase was dried and concentrated. For purification, the residue was chromatographed on silica gel (heptane/ethyl acetate 4:1). This gave initially 0.04 g (5.4% of theory) of 2-cyclobutyloxy-3-(2-thienyl)quinoxaline (O-alkylation product, colorless solid, melting point 103° C.) and then 0.41 g (59.9% of theory) of product as a colorless solid of melting point 110° C.

### Example E

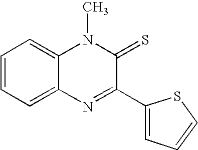

### 1-Methyl-3-(2-thienyl)-1,2-dihydroquinoxaline-2-thione

0.48 g (2 mmol) of 1-methyl-3-(2-thienyl)-1,2-dihydroquinoxalin-2-one (prepared analogously to example B from o-phenylenediamine and ethyl (2-thienyl)glyoxylate) and 0.41 mg (1 mmol) of Lawesson's reagent in 10 ml of xylene were heated under reflux for 10 hours. The mixture was concentrated and the residue was then chromatographed on silica gel (heptane/ethyl acetate 1:4). This gave 0.15 g of product (26.0% of theory) as an orange solid of melting point 113° C.

### Example F

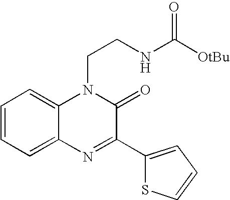

### 1-(2-Boc-aminoethyl)-3-(2-thienyl)-1,2-dihydroquinoxalin-2-one

A mixture of 5.1 g (0.022 mol) of 3-(2-thienyl)-1,2-dihydroquinoxalin-2-one (prepared analogously to example B from o-phenylenediamine and ethyl (2-thienyl)glyoxylate), 5.0 g (0.022 mol) of 2-(tert-butoxycarbonylamino)ethyl bromide [=2-(boc-amino)ethyl bromide] and 3.5 g (0.025 mol) of potassium carbonate was stirred at 90° C. for 7 hours. The solvent was removed under reduced pressure and the residue was then taken up in water/dichloromethane, and the organic phase was dried and concentrated. The residue was chromatographed on silica gel using heptane/ethyl acetate 7:3. This gave initially 0.86 g (9.9% of theory) of the O-alkylisomer (m.p. 144-145° C.) and then 1.59 g (18.2% of theory) of the desired product. Colorless crystals, m.p. 156-157° C.

### Example G

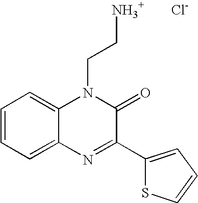

### 1-(2-Aminoethyl)-3-(2-thienyl)-1,2-dihydroquinoxalin-2-one hydrochloride

1.50 g (4 mmol) of the product from example F were dissolved in 20 ml of dioxane, and 2 ml of a 4M solution of hydrogen chloride in dioxane were added. The mixture was stirred at room temperature for 5 hours and under reflux for 5 hours. After cooling, the precipitated hydrochloride was filtered off with suction. This gave 1.08 g of product (82.6% of theory) as a colorless solid: m.p.: >250° C.

### Example H

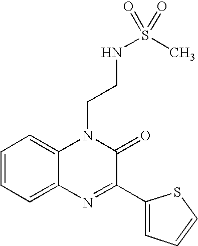

### 1-(2-Methylsulfonylaminoethyl)-3-(2-thienyl)-1,2-dihydroquinoxalin-2-one

At room temperature, a solution of 126 mg of methanesulfonyl chloride (1.1 mmol) in a little dichloromethane was added dropwise to a mixture of 300 mg of amine hydrochloride from example G (1.0 mmol) and 223 mg of triethylamine (2.2 mmol) in 10 ml of dichloromethane, and the mixture was stirred at room temperature for 6 hours. The reaction mixture was poured into water and the organic phase was dried and concentrated. The crude product was purified by chromatography on silica gel (heptane/silica gel 7:3). This gave 110 mg of product (47.0% of theory) as a colorless solid.

M.p.: 236-237° C.

### Example I

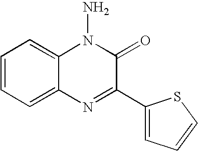

### 1-Amino-3-(2-thienyl)-1,2-dihydroquinoxalin-2-one

A mixture of 3.50 g (15 mmol) of 3-(2-thienyl)-1,2-dihydroquinoxalin-2-one (prepared analogously to example B from o-phenylenediamine and ethyl (2-thienyl)glyoxylate) and 4.77 g (42 mmol) of hydroxylamine O-sulfonic acid in a solution of 3.07 g (77 mmol) of sodium hydroxide was stirred at room temperature for 15 hours. The mixture was diluted with water and triturated with dichloromethane, and the organic phase was dried and concentrated. For purification, the residue was chromatographed on silica gel (heptane/ethyl acetate 4:1). This gave 0.36 g of product (9.2% of theory) as a colorless solid of melting point 164° C.

The tables below list, in an exemplary manner, a number of compounds of the formula I which can be obtained analogously to the examples above and the methods mentioned further above.

In tables 1 and 2:

| | | | |
|---|---|---|---|
| Bu = | butyl | Et = | ethyl |
| Me = | methyl | Ph = | phenyl |
| Pr = | propyl | Th = | thienyl |
| i = | iso | s = | secondary |
| t = | tertiary | | |

This applies correspondingly to composite terms such as
iPr=isopropyl
iBu=isobutyl
sBu=sec-butyl
tBu=tert-butyl

If an alkyl radical is listed in the tables without further specification, the radical in question is the straight-chain alkyl radical.

If the definition “H” is given for “(Y)ₙ”, this means the unsubstituted skeleton (n=0).
m.p.=melting point

.HCl=the hydrochloride of the parent compound

TABLE 1
Compounds of the formula (I-i)

| | |
|---|---|
| | (I-1) |
| 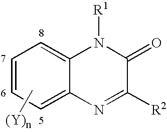 | |

| Ex. | (Y)ₙ | R² | R¹ | m.p. [° C.] |
|---|---|---|---|---|
| 1 | H | Me | H | 246-248 |
| 2 | H | Et | H | |
| 3 | H | Pr | H | |
| 4 | H | i-Pr | H | >250 |
| 5 | 7-OMe | i-Pr | H | 179 |
| 6 | 6,7-Cl₂ | i-Pr | H | >250 |
| 7 | 6,7-Me₂ | i-Pr | H | 268-269 |
| 8 | H | Bu | H | |
| 9 | H | i-Bu | H | |
| 10 | H | s-Bu | H | 184-185 |
| 11 | H | t-Bu | H | |
| 12 | H | cyclopropyl | H | |
| 13 | H | cyclobutyl | H | |
| 14 | H | cyclopentyl | H | 238 |
| 15 | H | cyclohexyl | H | >250 |
| 16 | H | cycloheptyl | H | |
| 17 | H | trifluoromethyl | H | 233-236 |
| 18 | H | benzyl | H | 199-201 |
| 19 | H | 1-phenylethyl | H | |
| 20 | H | 2-phenylethyl | H | 218-219 |
| 21 | H | 2-picolyl | H | |
| 22 | H | 3-picolyl | H | |
| 23 | H | 4-picolyl | H | |
| 24 | H | 2-thienylmethyl | H | |
| 25 | H | 3-thienylmethyl | H | |
| 26 | H | 4-chlorobenzyl | H | |
| 27 | H | 4-methylbenzyl | H | |
| 28 | H | 4-methoxybenzyl | H | |
| 29 | H | 3-indolylmethyl | H | 216-218 |
| 30 | H | Ph | H | 250-251 |
| 31 | 5-Me | Ph | H | |
| 32 | 6-Me | Ph | H | |
| 33 | 7-Me | Ph | H | |
| 34 | 8-Me | Ph | H | |
| 35 | 5-Cl | Ph | H | |
| 36 | 6-Cl | Ph | H | |
| 37 | 7-Cl | Ph | H | |
| 38 | 8-Cl | Ph | H | |
| 39 | 5-F | Ph | H | |
| 40 | 6-F | Ph | H | |
| 41 | 7-F | Ph | H | |
| 42 | 8-F | Ph | H | |
| 43 | 5-OMe | Ph | H | |
| 44 | 6-OMe | Ph | H | |
| 45 | 7-OMe | Ph | H | 199 |
| 46 | 8-OMe | Ph | H | |
| 47 | 5-CF₃ | Ph | H | |
| 48 | 6-CF₃ | Ph | H | >250 |
| 49 | 7-CF₃ | Ph | H | >250 |
| 50 | 8-CF₃ | Ph | H | |
| 51 | 6,7-Me₂ | Ph | H | >250 |
| 52 | 5,7-Me₂ | Ph | H | |
| 53 | 5,6-Me₂ | Ph | H | |
| 54 | 7,8-Me₂ | Ph | H | |
| 55 | 5,7-Me₂ | Ph | H | |
| 56 | 6,8-Me₂ | Ph | H | |
| 57 | 5,8-Me₂ | Ph | H | |
| 58 | 6,7-Cl₂ | Ph | H | >250 |
| 59 | 5,6-Cl₂ | Ph | H | |
| 60 | 5,7-Cl₂ | Ph | H | |
| 61 | 7,8-Cl₂ | Ph | H | |
| 62 | 6,8-Cl₂ | Ph | H | |
| 63 | 5,8-Cl₂ | Ph | H | |
| 64 | 6,7-(OMe)2 Ph | H | 258 | |
| 65 | 6,7-O—CF₂—O— | Ph | H | >291-292 |
| 66 | 6,7-F₂ | Ph | H | >250 |
| 67 | 5,7-F₂ | Ph | H | |
| 68 | 5,6-F₂ | Ph | H | |
| 69 | 7,8-F₂ | Ph | H | >250 |
| 70 | 6,8-F₂ | Ph | H | |
| 71 | 5,8-F₂ | Ph | H | |
| 72 | 6,7-(CF₃)₂ | Ph | H | |
| 73 | 5,7-(CF₃)₂ | Ph | H | >270 |
| 74 | 5,6-(CF₃)₂ | Ph | H | |
| 75 | 7,8-(CF₃)₂ | Ph | H | |
| 76 | 6,8-(CF₃)₂ | Ph | H | |
| 77 | 5,8-(CF₃)₂ | Ph | H | |
| 78 | 6-Cl, 7-F | Ph | H | |
| 79 | H | 2-Th | H | >250 |
| 80 | 5-Me | 2-Th | H | |
| 81 | 6-Me | 2-Th | H | >250 |
| 82 | 7-Me | 2-Th | H | |
| 83 | 8-Me | 2-Th | H | |
| 84 | 5-Cl | 2-Th | H | |
| 85 | 6-Cl | 2-Th | H | >250 |
| 86 | 7-Cl | 2-Th | H | |
| 87 | 8-Cl | 2-Th | H | |
| 88 | 5-F | 2-Th | H | |
| 89 | 6-F | 2-Th | H | |
| 90 | 7-F | 2-Th | H | >250 |
| 91 | 8-F | 2-Th | H | |
| 92 | 5-OMe | 2-Th | H | |
| 93 | 6-OMe | 2-Th | H | |
| 94 | 7-OMe | 2-Th | H | 215 |
| 95 | 8-0 Me | 2-Th | H | |
| 96 | 5-CF₃ | 2-Th | H | |
| 97 | 6-CF₃ | 2-Th | H | >250 |
| 98 | 7-CF₃ | 2-Th | H | >250 |
| 99 | 8-CF₃ | 2-Th | H | |
| 100 | 6,7-Me₂ | 2-Th | H | >250 |
| 101 | 5,7-Me₂ | 2-Th | H | |
| 102 | 5,6-Me₂ | 2-Th | H | |
| 103 | 7,8-Me₂ | 2-Th | H | |
| 104 | 5,7-Me₂ | 2-Th | H | |
| 105 | 6,8-Me₂ | 2-Th | H | |
| 106 | 5,8-Me₂ | 2-Th | H | |
| 107 | 6,7-Cl₂ | 2-Th | H | >250 |
| 108 | 5,7-Cl₂ | 2-Th | H | |
| 109 | 5,6-Cl₂ | 2-Th | H | |
| 110 | 7,8-Cl₂ | 2-Th | H | |
| 111 | 6,8-Cl₂ | 2-Th | H | |
| 112 | 5,8-Cl₂ | 2-Th | H | |
| 113 | 6,7-(OMe)₂ | 2-Th | H | >270 |
| 114 | 6,7-O—CF₂—O— | 2-Th | H | >270 |
| 115 | 6,7-F₂ | 2-Th | H | >250 |
| 116 | 5,7-F₂ | 2-Th | H | |
| 117 | 5,6-F₂ | 2-Th | H | |
| 118 | 7,8-F₂ | 2-Th | H | |
| 119 | 6,8-F₂ | 2-Th | H | |
| 120 | 5,8-F₂ | 2-Th | H | |
| 121 | 5,7-(CF₃)₂ | 2-Th | H | >250 |
| 122 | 6,7-(CF₃)₂ | 2-Th | H | |
| 123 | 5,6-(CF₃)₂ | 2-Th | H | |
| 124 | 7,8-(CF₃)₂ | 2-Th | H | |
| 125 | 6,8-(CF₃)₂ | 2-Th | H | |
| 126 | 5,8-(CF₃)₂ | 2-Th | H | |
| 127 | 6-Cl, 7-F | 2-Th | H | >250 |
| 128 | 6-COOMe | 2-Th | H | oil |
| 129 | 6-COOEt | 2-Th | H | oil |
| 130 | H | p-tolyl | H | 232 |
| 131 | H | m-tolyl | H | 220 |
| 132 | H | o-tolyl | H | |
| 133 | H | 4-C₆H₄-tBu | H | 213-214 |
| 134 | H | 4-C₆H₄Cl | H | 213-214 |
| 135 | H | 3-C₆H₄Cl | H | |
| 136 | H | 2-C₆H₄Cl | H | |
| 137 | H | 4-C₆H₄F | H | |
| 138 | H | 3-C₆H₄F | H | |
| 139 | H | 2-C₆H₄F | H | |
| 140 | H | 4-C₆H₄OMe | H | >250 |
| 141 | H | 3-C₆H₄OMe | H | |
| 142 | H | 2-C₆H₄OMe | H | |
| 143 | H | 4-C₆H₄CF₃ | H | |
| 144 | H | 3-C₆H₄CF₃ | H | 212 |
| 145 | H | 2-C₆H₄CF₃ | H | >250 |
| 146 | H | 2,3-C₆H₃Me₂ | H | |
| 147 | H | 2,4-C₆H₃Me₂ | H | 265 |
| 148 | H | 2,5-C₆H₃Me₂ | H | >250 |
| 149 | H | 2,6-C₆H₃Me₂ | H | |
| 150 | H | 3,4-C₆H₃Me₂ | H | 241 |
| 151 | H | 3,5-C₆H₃Me₂ | H | 202 |
| 152 | H | 2,3-C₆H₃Cl₂ | H | 204 |
| 153 | H | 2,4-C₆H₃Cl₂ | H | >250 |
| 154 | H | 2,5-C₆H₃Cl₂ | H | |
| 155 | H | 2,6-C₆H₃Cl₂ | H | |
| 156 | H | 3,4-C₆H₃Cl₂ | H | >250 |
| 157 | H | 3,5-C₆H₃Cl₂ | H | |
| 158 | H | 2,4,6-C₆H₂Me₃ | H | 202-204 |
| 159 | H | 3,4-C₆H₃(OMe)₂ | H | 238 |
| 160 | H | 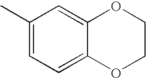 | H | >250 |
| 161 | H | 4-Cl, 2-F, 5-OMe-Ph | H | >250 |
| 162 | H | 3-Th | H | 268-269 |
| 163 | H | 2-furyl | H | 268 |
| 164 | H | 3-furyl | H | |
| 165 | H | 2-thiazolyl | H | |
| 166 | H | 4-thiazolyl | H | |
| 167 | H | 5-thiazolyl | H | |
| 168 | H | 2-pyridyl | H | |
| 169 | H | 3-pyridyl | H | |
| 170 | H | 4-pyridyl | H | |
| 171 | H | 3-Me-2-pyridyl | H | 244 |
| 172 | H | 4-Me-2-pyridyl | H | 170 |
| 173 | H | 5-Me-2-pyridyl | H | 232 |
| 174 | H | 6-Me-2-pyridyl | H | 254 |
| 175 | H | 2-Me-3-pyridyl | H | |
| 176 | H | 4-Me-3-pyridyl | H | |
| 177 | H | 5-Me-3-pyridyl | H | |
| 178 | H | 6-Me-3-pyridyl | H | |
| 179 | H | 2-Me-4-pyridyl | H | |
| 180 | H | 3-Me-4-pyridyl | H | |
| 181 | H | 3-Me-2-thienyl | H | 264 |
| 182 | H | 4-Me-2-thienyl | H | 253 |
| 183 | H | 5-Me-2-thienyl | H | >250 |
| 184 | H | 2-Me-3-thienyl | H | |
| 185 | H | 4-Me-3-thienyl | H | |
| 186 | H | 5-Me-3-thienyl | H | |
| 187 | H | 3,4-Me₂-2-thienyl | H | |
| 188 | H | 3,5-Me₂-2-thienyl | H | |
| 189 | H | 4,5-Me₂-2-thienyl | H | |
| 190 | H | 2,4-Me₂-3-thienyl | H | |
| 191 | H | 2,5-Me₂-3-thienyl | H | 221 |
| 192 | H | 4,5-Me₂-3-thienyl | H | |
| 193 | H | 3-Cl-2-thienyl | H | |
| 194 | H | 4-Cl-2-thienyl | H | |
| 195 | H | 5-Cl-2-thienyl | H | >250 |
| 196 | H | 2-Cl-3-thienyl | H | |
| 197 | H | 4-Cl-3-thienyl | H | |
| 198 | H | 5-Cl-3-thienyl | H | |
| 199 | H | 3,4-Cl₂-2-thienyl | H | |
| 200 | H | 3,5-Cl₂-2-thienyl | H | |
| 201 | H | 4,5-Cl₂-2-thienyl | H | |
| 202 | H | 2,4-Cl₂-3-thienyl | H | |
| 203 | H | 2,5-Cl₂-3-thienyl | H | >250 |
| 204 | H | 4,5-Cl₂-3-thienyl | H | |
| 205 | H | Me | Me | |
| 206 | H | Et | Me | |
| 207 | H | Pr | Me | |
| 208 | H | i-Pr | Me | |
| 209 | H | Bu | Me | |
| 210 | H | i-Bu | Me | |
| 211 | H | s-Bu | Me | |
| 212 | H | t-Bu | Me | |
| 213 | H | cyclopropyl | Me | |
| 214 | H | cyclobutyl | Me | |
| 215 | H | cyclopentyl | Me | 96 |
| 216 | H | cyclohexyl | Me | 183 |
| 217 | H | cycloheptyl | Me | |
| 218 | H | trifluoromethyl | Me | 137 |
| 219 | H | benzyl | Me | |
| 220 | H | 1-phenylethyl | Me | |
| 221 | H | 2-phenylethyl | Me | 93 |
| 222 | H | 2-picolyl | Me | |
| 223 | H | 3-picolyl | Me | |
| 224 | H | 4-picolyl | Me | |
| 225 | H | 2-thienylmethyl | Me | |
| 226 | H | 3-thienylmethyl | Me | |
| 227 | H | 4-chlorobenzyl | Me | |
| 228 | H | 4-methylbenzyl | Me | |
| 229 | H | 4-methoxybenzyl | Me | |
| 230 | H | 3-indolylmethyl | Me | |
| 231 | H | Ph | Me | 130-132 |
| 232 | 5-Me | Ph | Me | |
| 233 | 6-Me | Ph | Me | 135 |
| 234 | 7-Me | Ph | Me | |
| 235 | 8-Me | Ph | Me | |
| 236 | 5-Cl | Ph | Me | |
| 237 | 6-Cl | Ph | Me | |
| 238 | 7-Cl | Ph | Me | |
| 239 | 8-Cl | Ph | Me | |
| 240 | 5-F | Ph | Me | |
| 241 | 6-F | Ph | Me | |
| 242 | 7-F | Ph | Me | |
| 243 | 8-F | Ph | Me | |
| 244 | 5-OMe | Ph | Me | |
| 245 | 6-OMe | Ph | Me | |
| 246 | 7-OMe | Ph | Me | |
| 247 | 8-OMe | Ph | Me | |
| 248 | 5-CF₃ | Ph | Me | |
| 249 | 6-CF₃ | Ph | Me | 152 |
| 250 | 7-CF₃ | Ph | Me | 100 |
| 251 | 8-CF₃ | Ph | Me | |
| 252 | 6,7-Me₂ | Ph | Me | |
| 253 | 5,7-Me₂ | Ph | Me | |
| 254 | 5,6-Me₂ | Ph | Me | |
| 255 | 7,8-Me₂ | Ph | Me | |
| 256 | 5,7-Me₂ | Ph | Me | |
| 257 | 6,8-Me₂ | Ph | Me | |
| 258 | 5,8-Me₂ | Ph | Me | |
| 259 | 5,7-Cl₂ | Ph | Me | |
| 260 | 6,7-Cl₂ | Ph | Me | |
| 261 | 5,6-Cl₂ | Ph | Me | |
| 262 | 7,8-Cl₂ | Ph | Me | |
| 263 | 6,8-Cl₂ | Ph | Me | |
| 264 | 5,8-Cl₂ | Ph | Me | |
| 265 | 6,7-(OMe)₂ | Ph | Me | |
| 266 | 6,7-O—CF₂—O— | Ph | Me | 165 |
| 267 | 5,7-F₂ | Ph | Me | |
| 268 | 6,7-F₂ | Ph | Me | 141-142 |
| 269 | 5,6-F₂ | Ph | Me | |
| 270 | 7,8-F₂ | Ph | Me | >250 |
| 271 | 6,8-F₂ | Ph | Me | |
| 272 | 5,8-F₂ | Ph | Me | |
| 273 | 6-F,7-NMe₂ | Ph | Me | 168-169 |
| 274 | 5,7-(CF₃)₂ | Ph | Me | |
| 275 | 6,7-(CF₃)₂ | Ph | Me | |
| 276 | 5,6-(CF₃)₂ | Ph | Me | |
| 277 | 7,8-(CF₃)₂ | Ph | Me | |
| 278 | 6,8-(CF₃)₂ | Ph | Me | |
| 279 | 5,8-(CF₃)₂ | Ph | Me | |
| 280 | 6-Cl, 7-F | Ph | Me | |
| 281 | H | 2-Th | Me | 170-171 |
| 282 | 5-Me | 2-Th | Me | |
| 283 | 6-Me | 2-Th | Me | 194 |
| 284 | 7-Me | 2-Th | Me | |
| 285 | 8-Me | 2-Th | Me | |
| 286 | 5-Cl | 2-Th | Me | |
| 287 | 6-Cl | 2-Th | Me | |
| 288 | 7-Cl | 2-Th | Me | |
| 289 | 8-Cl | 2-Th | Me | |
| 290 | 5-F | 2-Th | Me | |
| 291 | 6-F | 2-Th | Me | |
| 292 | 7-F | 2-Th | Me | |
| 293 | 8-F | 2-Th | Me | |
| 294 | 5-OMe | 2-Th | Me | |
| 295 | 6-OMe | 2-Th | Me | |
| 296 | 7-OMe | 2-Th | Me | |
| 297 | 8-OMe | 2-Th | Me | |
| 298 | 5-CF₃ | 2-Th | Me | |
| 299 | 6-CF₃ | 2-Th | Me | 185 |
| 300 | 7-CF₃ | 2-Th | Me | |
| 301 | 8-CF₃ | 2-Th | Me | |
| 302 | 5,7-Me₂ | 2-Th | Me | |
| 303 | 6,7-Me₂ | 2-Th | Me | |
| 304 | 5,6-Me₂ | 2-Th | Me | |
| 305 | 7,8-Me₂ | 2-Th | Me | |
| 306 | 5,7-Me₂ | 2-Th | Me | |
| 307 | 6,8-Me₂ | 2-Th | Me | |
| 308 | 5,8-Me₂ | 2-Th | Me | |
| 309 | 5,7-Cl₂ | 2-Th | Me | |
| 310 | 6,7-Cl₂ | 2-Th | Me | |
| 311 | 5,6-Cl₂ | 2-Th | Me | |
| 312 | 7,8-Cl₂ | 2-Th | Me | |
| 313 | 6,8-Cl₂ | 2-Th | Me | |
| 314 | 5,8-Cl₂ | 2-Th | Me | |
| 315 | 6,7-(OMe)₂ | 2-Th | Me | |
| 316 | 6,7-O—CF₂—O— | 2-Th | Me | 241 |
| 317 | 5,7-F₂ | 2-Th | Me | |
| 318 | 6,7-F₂ | 2-Th | Me | |
| 319 | 5,6-F₂ | 2-Th | Me | |
| 320 | 7,8-F₂ | 2-Th | Me | |
| 321 | 6,8-F₂ | 2-Th | Me | |
| 322 | 5,8-F₂ | 2-Th | Me | |
| 323 | 5,7-(CF₃)₂ | 2-Th | Me | |
| 324 | 6,7-(CF₃)₂ | 2-Th | Me | |
| 325 | 5,6-(CF₃)₂ | 2-Th | Me | |
| 326 | 7,8-(CF₃)₂ | 2-Th | Me | |
| 327 | 6,8-(CF₃)₂ | 2-Th | Me | |
| 328 | 5,8-(CF₃)₂ | 2-Th | Me | |
| 329 | 6-Cl, 7-F | 2-Th | Me | |
| 330 | H | p-tolyl | Me | 149 |
| 331 | H | m-tolyl | Me | |
| 332 | H | o-tolyl | Me | 109 |
| 333 | H | 4-tBu | Me | |
| 334 | H | 4-C₆H₄Cl | Me | 178 |
| 335 | H | 3-C₆H₄Cl | Me | |
| 336 | H | 2-C₆H₄Cl | Me | |
| 337 | H | 4-C₆H₄F | Me | |
| 338 | H | 3-C₆H₄F | Me | |
| 339 | H | 2-C₆H₄F | Me | |
| 340 | H | 4-C₆H₄OMe | Me | 152 |
| 341 | H | 3-C₆H₄OMe | Me | |
| 342 | H | 2-C₆H₄OMe | Me | |
| 343 | H | 4-C₆H₄CF₃ | Me | 166 |
| 344 | H | 3-C₆H₄CF₃ | Me | 143 |
| 345 | H | 2-C₆H₄CF₃ | Me | |
| 346 | H | 2,3-C₆H₃Me₂ | Me | |
| 347 | H | 2,4-C₆H₃Me₂ | Me | |
| 348 | H | 2,5-C₆H₃Me₂ | Me | |
| 349 | H | 2,6-C₆H₃Me₂ | Me | 138 |
| 350 | H | 3,4-C₆H₃Me₂ | Me | 119 |
| 351 | H | 3,5-C₆H₃Me₂ | Me | |
| 352 | H | 2,4,6-C₆H₂Me₃ | Me | 186 |
| 353 | H | 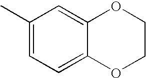 | Me | |
| 354 | H | 2,3-C₆H₃Cl₂ | Me | 188 |
| 355 | H | 2,4-C₆H₃Cl₂ | Me | |
| 356 | H | 2,5-C₆H₃Cl₂ | Me | |
| 357 | H | 2,6-C₆H₃Cl₂ | Me | |
| 358 | H | 3,4-C₆H₃Cl₂ | Me | 191 |
| 359 | H | 3,5-C₆H₃Cl₂ | Me | |
| 360 | H | 3,4-C₆H₃(OMe)₂ | Me | |
| 361 | H | 4-Cl, 2-F,5-OMe-phenyl | Me | 177 |
| 362 | H | 3-Th | Me | |
| 363 | H | 2-furyl | Me | 183 |
| 364 | H | 3-furyl | Me | |
| 365 | H | 2-thiazolyl | Me | |
| 366 | H | 4-thiazolyl | Me | |
| 367 | H | 5-thiazolyl | Me | |
| 368 | H | 2-pyridyl | Me | 108 |
| 369 | H | 3-pyridyl | Me | |
| 370 | H | 4-pyridyl | Me | |
| 371 | H | 3-Me-2-pyridyl | Me | 183 |
| 372 | H | 4-Me-2-pyridyl | Me | 134 |
| 373 | H | 5-Me-2-pyridyl | Me | 94 |
| 374 | H | 6-Me-2-pyridyl | Me | 183 |
| 375 | H | 2-Me-3-pyridyl | Me | |
| 376 | H | 4-Me-3-pyridyl | Me | |
| 377 | H | 5-Me-3-pyridyl | Me | |
| 378 | H | 6-Me-3-pyridyl | Me | |
| 379 | H | 2-Me-4-pyridyl | Me | |
| 380 | H | 3-Me-4-pyridyl | Me | |
| 381 | H | 3-Me-2-thienyl | Me | |
| 382 | H | 4-Me-2-thienyl | Me | 196 |
| 383 | H | 5-Me-2-thienyl | Me | 189 |
| 384 | H | 2-Me-3-thienyl | Me | |
| 385 | H | 4-Me-3-thienyl | Me | |
| 386 | H | 5-Me-3-thienyl | Me | |
| 387 | H | 3,4-Me₂-2-thienyl | Me | |
| 388 | H | 3,5-Me₂-2-thienyl | Me | |
| 389 | H | 4,5-Me₂-2-thienyl | Me | |
| 390 | H | 2,4-Me₂-3-thienyl | Me | |
| 391 | H | 2,5-Me₂-3-thienyl | Me | |
| 392 | H | 4,5-Me₂-3-thienyl | Me | |
| 393 | H | 3-Cl-2-thienyl | Me | |
| 394 | H | 4-Cl-2-thienyl | Me | |
| 395 | H | 5-Cl-2-thienyl | Me | 212-213 |
| 396 | H | 2-Cl-3-thienyl | Me | |
| 397 | H | 4-Cl-3-thienyl | Me | |
| 398 | H | 5-Cl-3-thienyl | Me | |
| 399 | H | 3,4-Cl₂-2-thienyl | Me | |
| 400 | H | 3,5-Cl₂-2-thienyl | Me | |
| 401 | H | 4,5-Cl₂-2-thienyl | Me | |
| 402 | H | 2,4-Cl₂-3-thienyl | Me | |
| 403 | H | 2,5-Cl₂-3-thienyl | Me | 169 |
| 404 | H | 4,5-Cl₂-3-thienyl | Me | |
| 405 | H | Me | (CH₂)₂NEt₂ | oil |
| 406 | H | Et | (CH₂)₂NEt₂ | |
| 407 | H | Pr | (CH₂)₂NEt₂ | |
| 408 | H | i-Pr | (CH₂)₂NEt₂ | oil |
| 409 | H | Bu | (CH₂)₂NEt₂ | |
| 410 | H | i-Bu | (CH₂)₂NEt₂ | |
| 411 | H | s-Bu | (CH₂)₂NEt₂ | |
| 412 | H | t-Bu | (CH₂)₂NEt₂ | oil |
| 413 | H | cyclopropyl | (CH₂)₂NEt₂ | |
| 414 | H | cyclobutyl | (CH₂)₂NEt₂ | |
| 415 | H | cyclopentyl | (CH₂)₂NEt₂ | oil |
| 416 | H | cyclohexyl | (CH₂)₂NEt₂ | oil |
| 417 | H | cycloheptyl | (CH₂)₂NEt₂ | |
| 418 | H | trifluoromethyl | (CH₂)₂NEt₂ | oil |
| 419 | H | benzyl | (CH₂)₂NEt₂ | |
| 420 | H | 1-phenylethyl | (CH₂)₂NEt₂ | |
| 421 | H | 2-phenylethyl | (CH₂)₂NEt₂ | oil |
| 422 | H | 2-picolyl | (CH₂)₂NEt₂ | |
| 423 | H | 3-picolyl | (CH₂)₂NEt₂ | |
| 424 | H | 4-picolyl | (CH₂)₂NEt₂ | |
| 425 | H | 2-thienylmethyl | (CH₂)₂NEt₂ | |
| 426 | H | 3-thienylmethyl | (CH₂)₂NEt₂ | |
| 427 | H | 4-chlorobenzyl | (CH₂)₂NEt₂ | |
| 428 | H | 4-methylbenzyl | (CH₂)₂NEt₂ | |
| 429 | H | 4-methoxybenzyl | (CH₂)₂NEt₂ | |
| 430 | H | 3-indolylmethyl | (CH₂)₂NEt₂ | |
| 431 | H | Ph | (CH₂)₂NEt₂ | oil |
| 432 | H | Ph | (CH₂)₂NEt₂.HCl | 134 |
| 433 | 5-Me | Ph | (CH₂)₂NEt₂ | |
| 434 | 6-Me | Ph | (CH₂)₂NEt₂ | oil |
| 435 | 6-Me | Ph | (CH₂)₂NEt₂.HCl | 223 |
| 436 | 7-Me | Ph | (CH₂)₂NEt₂ | 78 |
| 437 | 8-Me | Ph | (CH₂)₂NEt₂ | |
| 438 | 5-Cl | Ph | (CH₂)₂NEt₂ | |
| 439 | 6-Cl | Ph | (CH₂)₂NEt₂ | |
| 440 | 7-Cl | Ph | (CH₂)₂NEt₂ | |
| 441 | 8-Cl | Ph | (CH₂)₂NEt₂ | |
| 442 | 5-F | Ph | (CH₂)₂NEt₂ | |
| 443 | 6-F | Ph | (CH₂)₂NEt₂ | |
| 444 | 7-F | Ph | (CH₂)₂NEt₂ | |
| 445 | 8-F | Ph | (CH₂)₂NEt₂ | |
| 446 | 5-OMe | Ph | (CH₂)₂NEt₂ | |
| 447 | 6-OMe | Ph | (CH₂)₂NEt₂ | |
| 448 | 7-OMe | Ph | (CH₂)₂NEt₂ | |
| 449 | 8-OMe | Ph | (CH₂)₂NEt₂ | |
| 450 | 5-CF₃ | Ph | (CH₂)₂NEt₂ | |
| 451 | 6-CF₃ | Ph | (CH₂)₂NEt₂ | oil |
| 452 | 7-CF₃ | Ph | (CH₂)₂NEt₂ | |
| 453 | 8-CF₃ | Ph | (CH₂)₂NEt₂ | |
| 454 | 6,7-Me₂ | Ph | (CH₂)₂NEt₂ | 77 |
| 455 | 5,6-Me₂ | Ph | (CH₂)₂NEt₂ | |
| 456 | 7,8-Me₂ | Ph | (CH₂)₂NEt₂ | |
| 457 | 5,7-Me₂ | Ph | (CH₂)₂NEt₂ | |
| 458 | 6,8-Me₂ | Ph | (CH₂)₂NEt₂ | |
| 459 | 5,8-Me₂ | Ph | (CH₂)₂NEt₂ | |
| 460 | 6,7-Cl₂ | Ph | (CH₂)₂NEt₂ | 109 |
| 461 | 5,6-Cl₂ | Ph | (CH₂)₂NEt₂ | |
| 462 | 7,8-Cl₂ | Ph | (CH₂)₂NEt₂ | |
| 463 | 6,8-Cl₂ | Ph | (CH₂)₂NEt₂ | |
| 464 | 5,8-Cl₂ | Ph | (CH₂)₂NEt₂ | |
| 465 | 6,7-(OMe)₂ | Ph | (CH₂)₂NEt₂ | oil |
| 466 | 6,7-O—CF₂—O— | Ph | (CH₂)₂NEt₂ | 78-79 |
| 467 | 6,7-F₂ | Ph | (CH₂)₂NEt₂ | |
| 468 | 5,6-F₂ | Ph | (CH₂)₂NEt₂ | |
| 469 | 7,8-F₂ | Ph | (CH₂)₂NEt₂ | |
| 470 | 6,8-F₂ | Ph | (CH₂)₂NEt₂ | |
| 471 | 5,8-F₂ | Ph | (CH₂)₂NEt₂ | |
| 472 | 6,7-CF₃ | Ph | (CH₂)₂NEt₂ | |
| 473 | 5,6-CF₃ | Ph | (CH₂)₂NEt₂ | |
| 474 | 7,8-CF₃ | Ph | (CH₂)₂NEt₂ | |
| 475 | 6,8-CF₃ | Ph | (CH₂)₂NEt₂ | |
| 476 | 5,8-CF₃ | Ph | (CH₂)₂NEt₂ | |
| 477 | 6-Cl, 7-F | Ph | (CH₂)₂NEt₂ | |
| 478 | H | 2-Th | (CH₂)₂NEt₂ | oil |
| 479 | H | 2-Th | (CH₂)₂NEt₂.HCl | |
| 480 | 5-Me | 2-Th | (CH₂)₂NEt₂ | |
| 481 | 6-Me | 2-Th | (CH₂)₂NEt₂ | 86 |
| 482 | 6-Me | 2-Th | (CH₂)₂NEt₂.HCl | 275 |
| 483 | 7-Me | 2-Th | (CH₂)₂NEt₂ | oil |
| 484 | 8-Me | 2-Th | (CH₂)₂NEt₂ | |
| 485 | 5-Cl | 2-Th | (CH₂)₂NEt₂ | |
| 486 | 6-Cl | 2-Th | (CH₂)₂NEt₂ | oil |
| 487 | 7-Cl | 2-Th | (CH₂)₂NEt₂ | oil |
| 488 | 8-Cl | 2-Th | (CH₂)₂NEt₂ | |
| 489 | 5-F | 2-Th | (CH₂)₂NEt₂ | |
| 490 | 6-F | 2-Th | (CH₂)₂NEt₂ | |
| 491 | 7-F | 2-Th | (CH₂)₂NEt₂ | |
| 492 | 8-F | 2-Th | (CH₂)₂NEt₂ | |
| 493 | 5-OMe | 2-Th | (CH₂)₂NEt₂ | |
| 494 | 6-OMe | 2-Th | (CH₂)₂NEt₂ | |
| 495 | 7-OMe | 2-Th | (CH₂)₂NEt₂ | |
| 496 | 8-OMe | 2-Th | (CH₂)₂NEt₂ | |
| 497 | 5-CF₃ | 2-Th | (CH₂)₂NEt₂ | |
| 498 | 6-CF₃ | 2-Th | (CH₂)₂NEt₂ | solid |
| 499 | 7-CF₃ | 2-Th | (CH₂)₂NEt₂ | |
| 500 | 8-CF₃ | 2-Th | (CH₂)₂NEt₂ | |
| 501 | 6,7-Me₂ | 2-Th | (CH₂)₂NEt₂ | 129 |
| 502 | 5,6-Me₂ | 2-Th | (CH₂)₂NEt₂ | |
| 503 | 7,8-Me₂ | 2-Th | (CH₂)₂NEt₂ | |
| 504 | 5,7-Me₂ | 2-Th | (CH₂)₂NEt₂ | |
| 505 | 6,8-Me₂ | 2-Th | (CH₂)₂NEt₂ | |
| 506 | 5,8-Me₂ | 2-Th | (CH₂)₂NEt₂ | |
| 507 | 6,7-Cl₂ | 2-Th | (CH₂)₂NEt₂ | oil |
| 508 | 5,6-Cl₂ | 2-Th | (CH₂)₂NEt₂ | |
| 509 | 7,8-Cl₂ | 2-Th | (CH₂)₂NEt₂ | |
| 510 | 6,8-Cl₂ | 2-Th | (CH₂)₂NEt₂ | |
| 511 | 5,8-Cl₂ | 2-Th | (CH₂)₂NEt₂ | |
| 512 | 6,7-(OMe)₂ | 2-Th | (CH₂)₂NEt₂ | 127 |
| 513 | 6,7-O—CF₂—O— | 2-Th | (CH₂)₂NEt₂ | 70-71 |
| 514 | 6,7-F₂ | 2-Th | (CH₂)₂NEt₂ | |
| 515 | 5,6-F₂ | 2-Th | (CH₂)₂NEt₂ | |
| 516 | 7,8-F₂ | 2-Th | (CH₂)₂NEt₂ | |
| 517 | 6,8-F₂ | 2-Th | (CH₂)₂NEt₂ | |
| 518 | 5,8-F₂ | 2-Th | (CH₂)₂NEt₂ | |
| 519 | 6,7-CF₃ | 2-Th | (CH₂)₂NEt₂ | |
| 520 | 5,6-CF₃ | 2-Th | (CH₂)₂NEt₂ | |
| 521 | 7,8-CF₃ | 2-Th | (CH₂)₂NEt₂ | |
| 522 | 6,8-CF₃ | 2-Th | (CH₂)₂NEt₂ | |
| 523 | 5,8-CF₃ | 2-Th | (CH₂)₂NEt₂ | |
| 524 | 6-Cl, 7-F | 2-Th | (CH₂)₂NEt₂ | |
| 525 | 6-COOEt | 2-Th | (CH₂)₂NEt₂ | oil |
| 526 | H | p-tolyl | (CH₂)₂NEt₂ | oil |
| 527 | H | m-tolyl | (CH₂)₂NEt₂ | oil |
| 528 | H | o-tolyl | (CH₂)₂NEt₂ | |
| 529 | H | 4-tBu | (CH₂)₂NEt₂ | oil |
| 530 | H | 4-C₆H₄Cl | (CH₂)₂NEt₂ | oil |
| 531 | H | 3-C₆H₄Cl | (CH₂)₂NEt₂ | |
| 532 | H | 2-C₆H₄Cl | (CH₂)₂NEt₂ | |
| 533 | H | 4-C₆H₄F | (CH₂)₂NEt₂ | |
| 534 | H | 3-C₆H₄F | (CH₂)₂NEt₂ | |
| 535 | H | 2-C₆H₄F | (CH₂)₂NEt₂ | |
| 536 | H | 4-C₆H₄OMe | (CH₂)₂NEt₂ | oil |
| 537 | H | 3-C₆H₄OMe | (CH₂)₂NEt₂ | |
| 538 | H | 2-C₆H₄OMe | (CH₂)₂NEt₂ | |
| 539 | H | 4-C₆H₄CF₃ | (CH₂)₂NEt₂ | oil |
| 540 | H | 3-C₆H₄CF₃ | (CH₂)₂NEt₂ | |
| 541 | H | 2-C₆H₄CF₃ | (CH₂)₂NEt₂ | |
| 542 | H | 2,3-C₆H₃Me₂ | (CH₂)₂NEt₂ | |
| 543 | H | 2,4-C₆H₃Me₂ | (CH₂)₂NEt₂ | oil |
| 544 | H | 2,5-C₆H₃Me₂ | (CH₂)₂NEt₂ | |
| 545 | H | 2,6-C₆H₃Me₂ | (CH₂)₂NEt₂ | |
| 546 | H | 3,4-C₆H₃Me₂ | (CH₂)₂NEt₂ | oil |
| 546a | H | 3,4-C₆H₃Me₂ | (CH₂)₂NEt2 HOl | 137 |
| 547 | H | 3,5-C₆H₃Me₂ | (CH₂)₂NEt₂ | oil |
| 548 | H | 2,4,6-C₆H₂Me₃ | (CH₂)₂NEt₂ | oil |
| 549 | H | 3,4-C₆H₃(OMe)₂ | (CH₂)₂NEt₂ | 97 |
| 560 | H | 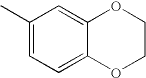 | (CH₂)₂NEt₂ | oil |
| 561 | H | 2,3-C₆H₃Cl₂ | (CH₂)₂NEt₂ | oil |
| 562 | H | 2,4-C₆H₃Cl₂ | (CH₂)₂NEt₂ | oil |
| 563 | H | 2,5-C₆H₃Cl₂ | (CH₂)₂NEt₂ | |
| 564 | H | 2,6-C₆H₃Cl₂ | (CH₂)₂NEt₂ | |
| 565 | H | 3,4-C₆H₃Cl₂ | (CH₂)₂NEt₂ | |
| 566 | H | 3,5-C₆H₃Cl₂ | (CH₂)₂NEt₂ | |
| 567 | H | 2,4,6-C₆H₂Cl₃ | (CH₂)₂NEt₂ | |
| 568 | H | 4-Cl, 2-F, 5-OMe-C₆H₂ | (CH₂)₂NEt₂ | |
| 569 | H | 3-Th | (CH₂)₂NEt₂ | oil |
| 570 | H | 2-furyl | (CH₂)₂NEt₂ | oil |
| 571 | H | 3-furyl | (CH₂)₂NEt₂ | |
| 572 | H | 2-thiazolyl | (CH₂)₂NEt₂ | |
| 573 | H | 4-thiazolyl | (CH₂)₂NEt₂ | |
| 574 | H | 5-thiazolyl | (CH₂)₂NEt₂ | |
| 575 | H | 2-pyridyl | (CH₂)₂NEt₂ | |
| 575a | H | 2-pyridyl | (CH₂)₂NEt₂.HC—CO—CF₃ | oil |
| 576 | H | 3-pyridyl | (CH₂)₂NEt₂ | |
| 577 | H | 4-pyridyl | (CH₂)₂NEt₂ | |
| 578 | H | 3-Me-2-pyridyl | (CH₂)₂NEt₂ | oil |
| 579 | H | 4-Me-2-pyridyl | (CH₂)₂NEt₂ | oil |
| 580 | H | 5-Me-2-pyridyl | (CH₂)₂NEt₂ | |
| 581 | H | 6-Me-2-pyridyl | (CH₂)₂NEt₂ | |
| 581a | H | 6-Me-2-pyridyl | (CH₂)₂NEt₂.HO—CO—CF₃ | oil |
| 582 | H | 2-Me-3-pyridyl | (CH₂)₂NEt₂ | |
| 583 | H | 4-Me-3-pyridyl | (CH₂)₂NEt₂ | |
| 584 | H | 5-Me-3-pyridyl | (CH₂)₂NEt₂ | |
| 585 | H | 6-Me-3-pyridyl | (CH₂)₂NEt₂ | |
| 586 | H | 2-Me-4-pyridyl | (CH₂)₂NEt₂ | |
| 587 | H | 3-Me-4-pyridyl | (CH₂)₂NEt₂ | |
| 588 | H | 2-Me-3-thienyl | (CH₂)₂NEt₂ | |
| 588a | H | 3-Me-2-thienyl | (CH₂)₂NEt₂ | oil |
| 589 | H | 4-Me-3-thienyl | (CH₂)₂NEt₂ | |
| 589a | H | 4-Me-2-thienyl | (CH₂)₂NEt₂ | |
| 590 | H | 5-Me-3-thienyl | (CH₂)₂NEt₂ | |
| 590a | H | 5-Me-2-thienyl | (CH₂)₂NEt₂ | |
| 591 | H | 3,4-Me₂-2-thienyl | (CH₂)₂NEt₂ | |
| 592 | H | 3,5-Me₂-2-thienyl | (CH₂)₂NEt₂ | |
| 593 | H | 4,5-Me₂-2-thienyl | (CH₂)₂NEt₂ | |
| 594 | H | 2,4-Me₂-3-thienyl | (CH₂)₂NEt₂ | |
| 595 | H | 2,5-Me₂-3-thienyl | (CH₂)₂NEt₂ | oil |
| 596 | H | 4,5-Me₂-3-thienyl | (CH₂)₂NEt₂ | |
| 597 | H | 3-Cl-2-thienyl | (CH₂)₂NEt₂ | |
| 598 | H | 4-Cl-2-thienyl | (CH₂)₂NEt₂ | |
| 599 | H | 5-Cl-2-thienyl | (CH₂)₂NEt₂ | |
| 600 | H | 2-Cl-3-thienyl | (CH₂)₂NEt₂ | |
| 601 | H | 4-Cl-3-thienyl | (CH₂)₂NEt₂ | |
| 602 | H | 5-Cl-3-thienyl | (CH₂)₂NEt₂ | |
| 603 | H | 3,4-Cl₂-2-thienyl | (CH₂)₂NEt₂ | |
| 604 | H | 3,5-Cl₂-2-thienyl | (CH₂)₂NEt₂ | |
| 605 | H | 4,5-Cl₂-2-thienyl | (CH₂)₂NEt₂ | |
| 606 | H | 2,4-Cl₂-3-thienyl | (CH₂)₂NEt₂ | |
| 607 | H | 2,5-Cl₂-3-thienyl | (CH₂)₂NEt₂ | |
| 608 | H | 4,5-Cl₂-3-thienyl | (CH₂)₂NEt₂ | |
| 609 | H | Me | CH₂COOMe | 135-136 |
| 610 | H | Et | CH₂COOMe | |
| 611 | H | Pr | CH₂COOMe | |
| 612 | H | i-Pr | CH₂COOMe | oil |
| 613 | 7-OMe | i-Pr | CH₂COOMe | |
| 614 | 6,7-Cl₂ | i-Pr | CH₂COOMe | |
| 615 | 6,7-Me₂ | i-Pr | CH₂COOMe | |
| 616 | H | Bu | CH₂COOMe | |
| 617 | H | i-Bu | CH₂COOMe | |
| 618 | H | s-Bu | CH₂COOMe | |
| 619 | H | t-Bu | CH₂COOMe | |
| 620 | H | cyclopropyl | CH₂COOMe | |
| 621 | H | cyclobutyl | CH₂COOMe | |
| 622 | H | cyclopentyl | CH₂COOMe | |
| 623 | H | cyclohexyl | CH₂COOMe | |
| 624 | H | cycloheptyl | CH₂COOMe | |
| 625 | H | trifluoromethyl | CH₂COOMe | |
| 626 | H | benzyl | CH₂COOMe | |
| 627 | H | 1-phenylethyl | CH₂COOMe | |
| 628 | H | 2-phenylethyl | CH₂COOMe | |
| 629 | H | 2-picolyl | CH₂COOMe | |
| 630 | H | 3-picolyl | CH₂COOMe | |
| 631 | H | 4-picolyl | CH₂COOMe | |
| 632 | H | 2-thienylmethyl | CH₂COOMe | |
| 633 | H | 3-thienylmethyl | CH₂COOMe | |
| 634 | H | 4-chlorobenzyl | CH₂COOMe | |
| 635 | H | 4-methylbenzyl | CH₂COOMe | |
| 636 | H | 4-methoxybenzyl | CH₂COOMe | |
| 637 | H | 3-indolylmethyl | CH₂COOMe | |
| 638 | H | Ph | CH₂COOMe | oil |
| 639 | 5-Me | Ph | CH₂COOMe | |
| 640 | 6-Me | Ph | CH₂COOMe | |
| 641 | 7-Me | Ph | CH₂COOMe | |
| 642 | 8-Me | Ph | CH₂COOMe | |
| 643 | 5-Cl | Ph | CH₂COOMe | |
| 644 | 6-Cl | Ph | CH₂COOMe | |
| 645 | 7-Cl | Ph | CH₂COOMe | |
| 646 | 8-Cl | Ph | CH₂COOMe | |
| 647 | 5-F | Ph | CH₂COOMe | |
| 648 | 6-F | Ph | CH₂COOMe | |
| 649 | 7-F | Ph | CH₂COOMe | |
| 650 | 8-F | Ph | CH₂COOMe | |
| 651 | 5-OMe | Ph | CH₂COOMe | |
| 652 | 6-OMe | Ph | CH₂COOMe | |
| 653 | 7-OMe | Ph | CH₂COOMe | |
| 654 | 8-OMe | Ph | CH₂COOMe | |
| 655 | 5-CF₃ | Ph | CH₂COOMe | |
| 656 | 6-CF₃ | Ph | CH₂COOMe | |
| 657 | 7-CF₃ | Ph | CH₂COOMe | |
| 658 | 8-CF₃ | Ph | CH₂COOMe | |
| 659 | 6,7-Me₂ | Ph | CH₂COOMe | 185 |
| 660 | 5,7-Me₂ | Ph | CH₂COOMe | |
| 661 | 5,6-Me₂ | Ph | CH₂COOMe | |
| 662 | 7,8-Me₂ | Ph | CH₂COOMe | |
| 663 | 5,7-Me₂ | Ph | CH₂COOMe | |
| 664 | 6,8-Me₂ | Ph | CH₂COOMe | |
| 665 | 5,8-Me₂ | Ph | CH₂COOMe | |
| 666 | 6,7-Cl₂ | Ph | CH₂COOMe | |
| 667 | 5,6-Cl₂ | Ph | CH₂COOMe | |
| 668 | 5,7-Cl₂ | Ph | CH₂COOMe | |
| 669 | 7,8-Cl₂ | Ph | CH₂COOMe | |
| 670 | 6,8-Cl₂ | Ph | CH₂COOMe | |
| 671 | 5,8-Cl₂ | Ph | CH₂COOMe | |
| 672 | 6,7-(OMe)₂ | Ph | CH₂COOMe | |
| 673 | 6,7-O—CF₂—O— | Ph | CH₂COOMe | |
| 674 | 6,7-F₂ | Ph | CH₂COOMe | |
| 675 | 5,7-F₂ | Ph | CH₂COOMe | |
| 676 | 5,6-F₂ | Ph | CH₂COOMe | |
| 677 | 7,8-F₂ | Ph | CH₂COOMe | |
| 678 | 6,8-F₂ | Ph | CH₂COOMe | |
| 679 | 5,8-F₂ | Ph | CH₂COOMe | |
| 680 | 6,7-(CF₃)₂ | Ph | CH₂COOMe | |
| 681 | 5,7-(CF₃)₂ | Ph | CH₂COOMe | |
| 682 | 5,6-(CF₃)₂ | Ph | CH₂COOMe | |
| 683 | 7,8-(CF₃)₂ | Ph | CH₂COOMe | |
| 684 | 6,8-(CF₃)₂ | Ph | CH₂COOMe | |
| 685 | 5,8-(CF₃)₂ | Ph | CH₂COOMe | |
| 686 | 6-Cl, 7-F | Ph | CH₂COOMe | |
| 687 | H | 2-Th | CH₂COOMe | oil |
| 688 | 5-Me | 2-Th | CH₂COOMe | oil |
| 689 | 6-Me | 2-Th | CH₂COOMe | oil |
| 690 | 7-Me | 2-Th | CH₂COOMe | |
| 691 | 8-Me | 2-Th | CH₂COOMe | oil |
| 692 | 5-Cl | 2-Th | CH₂COOMe | |
| 693 | 6-Cl | 2-Th | CH₂COOMe | |
| 694 | 7-Cl | 2-Th | CH₂COOMe | |
| 695 | 8-Cl | 2-Th | CH₂COOMe | |
| 696 | 5-F | 2-Th | CH₂COOMe | |
| 697 | 6-F | 2-Th | CH₂COOMe | |
| 698 | 7-F | 2-Th | CH₂COOMe | |
| 699 | 8-F | 2-Th | CH₂COOMe | |
| 700 | 5-OMe | 2-Th | CH₂COOMe | |
| 701 | 6-OMe | 2-Th | CH₂COOMe | |
| 702 | 7-OMe | 2-Th | CH₂COOMe | |
| 703 | 8-OMe | 2-Th | CH₂COOMe | |
| 704 | 5-CF₃ | 2-Th | CH₂COOMe | |
| 705 | 6-CF₃ | 2-Th | CH₂COOMe | |
| 706 | 7-CF₃ | 2-Th | CH₂COOMe | |
| 707 | 8-CF₃ | 2-Th | CH₂COOMe | |
| 708 | 6,7-Me₂ | 2-Th | CH₂COOMe | oil |
| 709 | 5,7-Me₂ | 2-Th | CH₂COOMe | |
| 710 | 5,6-Me₂ | 2-Th | CH₂COOMe | |
| 711 | 7,8-Me₂ | 2-Th | CH₂COOMe | |
| 712 | 5,7-Me₂ | 2-Th | CH₂COOMe | |
| 713 | 6,8-Me₂ | 2-Th | CH₂COOMe | |
| 714 | 5,8-Me₂ | 2-Th | CH₂COOMe | |
| 715 | 6,7-Cl₂ | 2-Th | CH₂COOMe | |
| 716 | 5,7-Cl₂ | 2-Th | CH₂COOMe | |
| 717 | 5,6-Cl₂ | 2-Th | CH₂COOMe | |
| 718 | 7,8-Cl₂ | 2-Th | CH₂COOMe | |
| 719 | 6,8-Cl₂ | 2-Th | CH₂COOMe | |
| 720 | 5,8-Cl₂ | 2-Th | CH₂COOMe | |
| 721 | 6,7-(OMe)₂ | 2-Th | CH₂COOMe | |
| 722 | 6,7-O—CF₂—O— | 2-Th | CH₂COOMe | |
| 723 | 6,7-F₂ | 2-Th | CH₂COOMe | |
| 724 | 5,7-F₂ | 2-Th | CH₂COOMe | |
| 725 | 5,6-F₂ | 2-Th | CH₂COOMe | |
| 726 | 7,8-F₂ | 2-Th | CH₂COOMe | |
| 727 | 6,8-F₂ | 2-Th | CH₂COOMe | |
| 728 | 5,8-F₂ | 2-Th | CH₂COOMe | |
| 729 | 5,7-(CF₃)₂ | 2-Th | CH₂COOMe | |
| 730 | 6,7-(CF₃)₂ | 2-Th | CH₂COOMe | |
| 731 | 5,6-(CF₃)₂ | 2-Th | CH₂COOMe | |
| 732 | 7,8-(CF₃)₂ | 2-Th | CH₂COOMe | |
| 733 | 6,8-(CF₃)₂ | 2-Th | CH₂COOMe | |
| 734 | 5,8-(CF₃)₂ | 2-Th | CH₂COOMe | |
| 735 | 6-Cl, 7-F | 2-Th | CH₂COOMe | |
| 736 | 7-COOMe | 2-Th | CH₂COOMe | |
| 737 | 7-COOEt | 2-Th | CH₂COOMe | |
| 738 | H | p-tolyl | CH₂COOMe | |
| 739 | H | m-tolyl | CH₂COOMe | |
| 740 | H | o-tolyl | CH₂COOMe | |
| 741 | H | 4-tBu | CH₂COOMe | |
| 742 | H | 4-C₆H₄Cl | CH₂COOMe | |
| 743 | H | 3-C₆H₄Cl | CH₂COOMe | |
| 744 | H | 2-C₆H₄Cl | CH₂COOMe | |
| 745 | H | 4-C₆H₄F | CH₂COOMe | |
| 746 | H | 3-C₆H₄F | CH₂COOMe | |
| 747 | H | 2-C₆H₄F | CH₂COOMe | |
| 748 | H | 4-C₆H₄OMe | CH₂COOMe | |
| 749 | H | 3-C₆H₄OMe | CH₂COOMe | |
| 750 | H | 2-C₆H₄OMe | CH₂COOMe | |
| 751 | H | 4-C₆H₄CF₃ | CH₂COOMe | |
| 752 | H | 3-C₆H₄CF₃ | CH₂COOMe | |
| 753 | H | 2-C₆H₄CF₃ | CH₂COOMe | |
| 754 | H | 2,3-C₆H₃Me₂ | CH₂COOMe | |
| 755 | H | 2,4-C₆H₃Me₂ | CH₂COOMe | |
| 756 | H | 2,5-C₆H₃Me₂ | CH₂COOMe | |
| 757 | H | 2,6-C₆H₃Me₂ | CH₂COOMe | |
| 758 | H | 3,4-C₆H₃Me₂ | CH₂COOMe | |
| 759 | H | 3,5-C₆H₃Me₂ | CH₂COOMe | |
| 760 | H | 2,3-C₆H₃Cl₂ | CH₂COOMe | |
| 761 | H | 2,4-C₆H₃Cl₂ | CH₂COOMe | |
| 762 | H | 2,5-C₆H₃Cl₂ | CH₂COOMe | |
| 763 | H | 2,6-C₆H₃Cl₂ | CH₂COOMe | |
| 764 | H | 3,4-C₆H₃Cl₂ | CH₂COOMe | |
| 765 | H | 3,5-C₆H₃Cl₂ | CH₂COOMe | |
| 766 | H | 2,4,6-C₆H₂Me₃ | CH₂COOMe | |
| 767 | H | 3,4-C₆H₃(OMe)₂ | CH₂COOMe | |
| 768 | H | 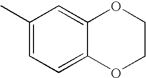 | CH₂COOMe | |
| 769 | H | 4-Cl, 2-F, 5-OMe-Ph | CH₂COOMe | |
| 770 | H | 3-Th | CH₂COOMe | |
| 771 | H | 2-furyl | CH₂COOMe | |
| 772 | H | 3-furyl | CH₂COOMe | |
| 773 | H | 2-thiazolyl | CH₂COOMe | |
| 774 | H | 4-thiazolyl | CH₂COOMe | |
| 775 | H | 5-thiazolyl | CH₂COOMe | |
| 776 | H | 2-pyridyl | CH₂COOMe | |
| 777 | H | 3-pyridyl | CH₂COOMe | |
| 778 | H | 4-pyridyl | CH₂COOMe | |
| 779 | H | 3-Me-2-pyridyl | CH₂COOMe | |
| 780 | H | 4-Me-2-pyridyl | CH₂COOMe | |
| 781 | H | 5-Me-2-pyridyl | CH₂COOMe | |
| 782 | H | 6-Me-2-pyridyl | CH₂COOMe | |
| 783 | H | 2-Me-3-pyridyl | CH₂COOMe | |
| 784 | H | 4-Me-3-pyridyl | CH₂COOMe | |
| 785 | H | 5-Me-3-pyridyl | CH₂COOMe | |
| 786 | H | 6-Me-3-pyridyl | CH₂COOMe | |
| 787 | H | 2-Me-4-pyridyl | CH₂COOMe | |
| 788 | H | 3-Me-4-pyridyl | CH₂COOMe | |
| 789 | H | 3-Me-2-thienyl | CH₂COOMe | |
| 790 | H | 4-Me-2-thienyl | CH₂COOMe | |
| 791 | H | 5-Me-2-thienyl | CH₂COOMe | |
| 792 | H | 2-Me-3-thienyl | CH₂COOMe | |
| 793 | H | 4-Me-3-thienyl | CH₂COOMe | |
| 794 | H | 5-Me-3-thienyl | CH₂COOMe | |
| 795 | H | 3,4-Me₂-2-thienyl | CH₂COOMe | |
| 796 | H | 3,5-Me₂-2-thienyl | CH₂COOMe | |
| 797 | H | 4,5-Me₂-2-thienyl | CH₂COOMe | |
| 798 | H | 2,4-Me₂-3-thienyl | CH₂COOMe | |
| 799 | H | 2,5-Me₂-3-thienyl | CH₂COOMe | |
| 800 | H | 4,5-Me₂-3-thienyl | CH₂COOMe | |
| 801 | H | 3-Cl-2-thienyl | CH₂COOMe | |
| 802 | H | 4-Cl-2-thienyl | CH₂COOMe | |
| 803 | H | 5-Cl-2-thienyl | CH₂COOMe | |
| 804 | H | 2-Cl-3-thienyl | CH₂COOMe | |
| 805 | H | 4-Cl-3-thienyl | CH₂COOMe | |
| 806 | H | 5-Cl-3-thienyl | CH₂COOMe | |
| 807 | H | 3,4-Cl₂-2-thienyl | CH₂COOMe | |
| 808 | H | 3,5-Cl₂-2-thienyl | CH₂COOMe | |
| 809 | H | 4,5-Cl₂-2-thienyl | CH₂COOMe | |
| 810 | H | 2,4-Cl₂-3-thienyl | CH₂COOMe | |
| 811 | H | 2,5-Cl₂-3-thienyl | CH₂COOMe | |
| 812 | H | 4,5-Cl₂-3-thienyl | CH₂COOMe | |
| 813 | H | Ph | CH₂COOEt | 116 |
| 814 | H | Ph | CH₂COOPr | 102 |
| 815 | H | Ph | CH₂CH₂COOiPr | 145 |
| 816 | H | Ph | CH₂COOBu | |
| 817 | H | Ph | CH₂COOiBu | |
| 818 | H | Ph | CH₂COOsBu | |
| 819 | H | Ph | CH₂COOtBu | |
| 820 | H | Ph | CH₂COOH | 228 |
| 821 | H | Ph | CH(Me)COOMe | |
| 822 | H | Ph | CH(Me)COOEt | |
| 823 | H | Ph | (CH₂)₂COOMe | |
| 824 | H | Ph | (CH₂)₂COOEt | |
| 825 | H | Ph | (CH₂)₃COOMe | |
| 826 | H | Ph | (CH₂)₃COOEt | |
| 827 | H | Ph | (CH₂)₃COOH | |
| 828 | H | Ph | CH₂CONMe₂ | |
| 829 | H | Ph | CH₂CONEt₂ | 149-150 |
| 830 | H | Ph | CH₂CONPr₂ | |
| 831 | H | Ph | CH₂CONiPr₂ | |
| 832 | H | Ph | CH₂CONMeEt | |
| 833 | H | Ph | 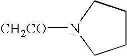 | |
| 834 | H | Ph | 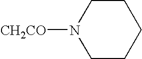 | |
| 835 | H | Ph | 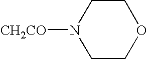 | 207 |
| 836 | H | Ph | 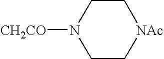 | |
| 837 | H | Ph | CH₂CONMe(OMe) | 219-220 |
| 838 | H | Ph | CH₂CONMePh | |
| 839 | H | Ph | CH₂CONHMe | |
| 840 | H | Ph | CH₂CONHEt | |
| 841 | H | Ph | CH₂CONHPr | |
| 842 | H | Ph | CH₂CONHiPr | |
| 843 | H | Ph | CH₂CONHBu | |
| 844 | H | Ph | CH₂CONHiBu | |
| 845 | H | Ph | CH₂CONHsBu | |
| 846 | H | Ph | CH₂CONHtBu | |
| 847 | H | Ph | CH₂CONHPh | |
| 848 | H | Ph | CH₂COMe | 159-161 |
| 849 | H | Ph | CH(CH₃)COMe | |
| 850 | H | Ph | CH₂COCF₃ | |
| 851 | H | Ph | CH₂COEt | |
| 852 | H | Ph | CH₂COPr | |
| 853 | H | Ph | CH₂COiPr | |
| 854 | H | Ph | CH₂COtBu | |
| 855 | H | Ph | CH₂OMe | oil |
| 856 | H | Ph | CH₂OEt | oil |
| 857 | H | Ph | (CH₂)₂OMe | oil |
| 858 | H | Ph | (CH₂)₂OEt | oil |
| 859 | H | Ph | (CH₂)₂OPr | |
| 860 | H | Ph | (CH₂)₂OiPr | |
| 861 | H | Ph | (CH₂)₂OtBu | oil |
| 862 | H | Ph | (CH₂)₂O(CH₂)₂OMe | oil |
| 863 | H | Ph | (CH₂)₂O(CH₂)₂OEt | |
| 864 | H | Ph | (CH₂)₂OCH₂CF₃ | |
| 865 | H | Ph | (CH₂)₂OPh | |
| 866 | H | Ph | (CH₂)₃OH | |
| 867 | H | Ph | (CH₂)₃OMe | |
| 868 | H | Ph | (CH₂)₃OEt | |
| 869 | H | Ph | (CH₂)₃OPr | |
| 870 | H | Ph | (CH₂)₃OiPr | |
| 871 | H | Ph | (CH₂)₃OtBu | |
| 872 | H | Ph | (CH₂)₂(OMe)₂ | |
| 873 | H | Ph | (CH₂)₂(OEt)₂ | |
| 874 | H | Ph | CH₂(OMe)₂ | |
| 875 | H | Ph | CH₂(OEt)₂ | |
| 876 | H | Ph | CH₂CH(OMe)CH₂OMe | 225 |
| 877 | H | Ph | CH₂SMe | 94 |
| 878 | H | Ph | (CH₂)₂SMe | |
| 879 | H | Ph | (CH₂)₂SEt | |
| 880 | H | Ph | (CH₂)₂SPr | |
| 881 | H | Ph | (CH₂)₂SiPr | |
| 882 | H | Ph | (CH₂)₂StBu | |
| 883 | H | Ph | (CH₂)₂SPh | oil |
| 884 | H | Ph | (CH₂)₂SCH₂CF₃ | |
| 885 | H | Ph | (CH₂)₂S-cyclopentyl | |
| 886 | H | Ph | 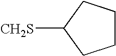 | |
| 887 | H | Ph | (CH₂)₂S(O)Me | |
| 888 | H | Ph | (CH₂)₂S(O)₂Me | |
| 889 | H | Ph | (CH₂)₂S(O)Et | |
| 890 | H | Ph | (CH₂)₂S(O)₂Et | |
| 891 | H | Ph | (CH₂)₂S(O)Ph | |
| 892 | H | Ph | (CH₂)₂S(O)₂Ph | 153-154 |
| 893 | H | Ph | (CH₂)₂NH₂ | |
| 893a | H | Ph | (CH₂)₂NH₂.HCl | 256 |
| 894 | H | Ph | (CH₂)₂NHMe.HCl | |
| 895 | H | Ph | (CH₂)₂NHEt | |
| 896 | H | Ph | (CH₂)₂NHPr | |
| 897 | H | Ph | (CH₂)₂NHiPr | |
| 898 | H | Ph | (CH₂)₃NH₂ | |
| 899 | H | Ph | (CH₂)₂NMe₂ | |
| 900 | H | Ph | (CH₂)₂NMe₂.HCl | |
| 901 | H | Ph | (CH₂)₂NPr₂ | |
| 902 | H | Ph | (CH₂)₂NiPr₂ | |
| 903 | H | Ph | 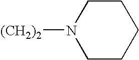 | |
| 904 | H | Ph | 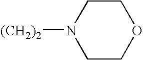 | 95 |
| 905 | H | Ph | 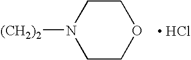 | 190 |
| 906 | H | Ph | 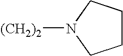 | |
| 907 | H | Ph | (CH₂)₃NMe₂ | oil |
| 908 | H | Ph | (CH₂)₃NMe₂.HCl | 208-209 |
| 909 | H | Ph | (CH₂)₃NEt₂ | 141 |
| 910 | H | Ph | (CH₂)₃NEt₂.HCl | |
| 911 | H | Ph | 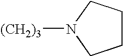 | |
| 912 | H | Ph | 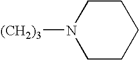 | |
| 913 | H | Ph | 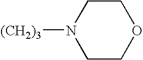 | oil |
| 914 | H | Ph | CH₂CH(CH₃)CH₂NMe₂ | |
| 915 | H | Ph | CH(CH₃)CH₂NMe₂ | |
| 916 | H | Ph | CH₂CH(CH₃)NMe₂ | 74-75 |
| 917 | H | Ph | CH₂CH(CH₃)NMe₂HCl | 198-199 |
| 918 | H | Ph | 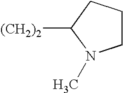 | |
| 919 | H | Ph | 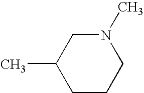 | |
| 920 | H | Ph | 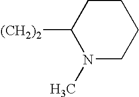 | |
| 921 | H | Ph | (CH₂)₂NHAc | 203 |
| 922 | H | Ph | (CH₂)₂NHCOEt | |
| 923 | H | Ph | (CH₂)₂NHCOPr | |
| 924 | H | Ph | (CH₂)₂NHCOiPr | |
| 925 | H | Ph | (CH₂)₂NHCOBu | |
| 926 | H | Ph | (CH₂)₂NHCOtBu | |
| 927 | H | Ph | (CH₂)₂NHCOPh | |
| 928 | H | Ph | (CH₂)₂NHCOOMe | 151 |
| 929 | H | Ph | (CH₂)₂NHCOOEt | |
| 930 | H | Ph | (CH₂)₂NHCOOPr | |
| 931 | H | Ph | (CH₂)₂NHCOOiPr | |
| 932 | H | Ph | (CH₂)₂NHCOOtBu | 137 |
| 933 | 7-CF₃ | Ph | (CH₂)₂NHCOOtBu | 150-154 |
| 934 | H | Ph | (CH₂)₂NHCONHMe | |
| 935 | H | Ph | (CH₂)₂NHCONMe₂ | |
| 936 | H | Ph | (CH₂)₂NHCONHEt | |
| 937 | H | Ph | (CH₂)₂NHCONEt₂ | |
| 938 | H | Ph | 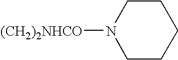 | |
| 939 | H | Ph | 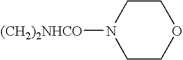 | |
| 940 | H | Ph | 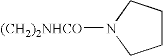 | |
| 941 | H | Ph | (CH₂)₂NMeCONMe₂ | |
| 942 | H | Ph | (CH₂)₂NMeCONEt₂ | |
| 943 | H | Ph | (CH₂)₂NHSO₂Me | 170 |
| 944 | H | Ph | (CH₂)₂NMeSO₂Me | |
| 945 | H | Ph | (CH₂)₂NHSO₂Ph | |
| 946 | H | Ph | (CH₂)₂NMeSO₂Ph | |
| 947 | H | Ph | (CH₂)₂OH | 127-129 |
| 948 | 7-NO₂ | Ph | (CH₂)₂OH | 147-150 |
| 949 | H | Ph | (CH₂)₂OAc | 113-114 |
| 950 | H | Ph | (CH₂)₂COOPr | |
| 951 | H | Ph | (CH₂)₂COOiPr | |
| 952 | H | Ph | (CH₂)₂COOBu | |
| 953 | H | Ph | (CH₂)₂COOtBu | |
| 954 | H | Ph | (CH₂)₂OCOOMe | |
| 955 | H | Ph | (CH₂)₂COOCEt | |
| 956 | H | Ph | (CH₂)₂COOCPr | |
| 957 | H | Ph | (CH₂)₂COOCiPr | |
| 958 | H | Ph | (CH₂)₂COOCBu | |
| 959 | H | Ph | (CH₂)₂COOCtBu | |
| 960 | H | Ph | (CH₂)₂OCONMe₂ | |
| 961 | H | Ph | (CH₂)₂COONEt₂ | |
| 962 | H | Ph | 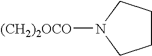 | |
| 963 | H | Ph | 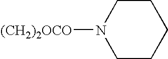 | |
| 964 | H | Ph | 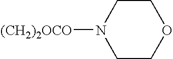 | |
| 965 | H | Ph | CH₂CH(OH)Me | 104-105 |
| 966 | H | Ph | CH₂OH(OH)CH₂OH | |
| 967 | H | Ph | Et | |
| 968 | H | Ph | Pr | |
| 969 | H | Ph | iPr | oil |
| 970 | H | Ph | Bu | |
| 971 | H | Ph | iBu | |
| 972 | H | Ph | sBu | |
| 973 | H | Ph | tBu | |
| 974 | H | Ph | CH₂CH₂F | |
| 975 | H | Ph | CH₂CHF₂ | |
| 976 | H | Ph | CH₂CF₃ | 145-146 |
| 977 | H | Ph | (CH₂)₂CF₃ | |
| 978 | H | Ph | CH₂-cyclopropyl | |
| 979 | H | Ph | CH₂-cyclobutyl | |
| 980 | H | Ph | CH₂-cyclopentyl | |
| 981 | H | Ph | CH₂-cyclohexyl | |
| 982 | H | Ph | benzyl | 151-152 |
| 983 | H | Ph | 2-furfuryl | |
| 984 | H | Ph | 3-furfuryl | |
| 985 | H | Ph | 2-thienylmethyl | |
| 986 | H | Ph | 3-thienylmethyl | |
| 987 | H | Ph | 2-(5-chlorothienyl)methyl | 134 |
| 988 | H | Ph | 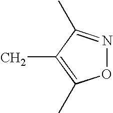 | 124-125 |
| 989 | H | Ph | 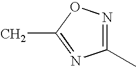 | 167 |
| 990 | H | Ph | 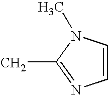 | |
| 991 | H | Ph | 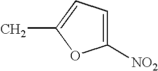 | |
| 992 | H | Ph | 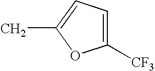 | |
| 993 | H | Ph | 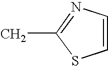 | |
| 994 | H | Ph | 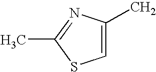 | |
| 995 | H | Ph | 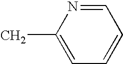 | |
| 996 | H | Ph | 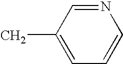 | |
| 997 | H | Ph | 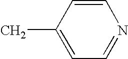 | |
| 998 | H | Ph | 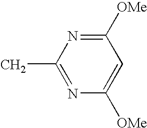 | 129-131 |
| 999 | H | Ph | 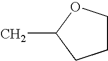 | 76-77 |
| 1000 | H | Ph | 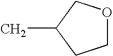 | 118 |
| 1001 | H | Ph | 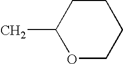 | 120-121 |
| 1002 | H | Ph | 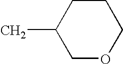 | |
| 1003 | H | Ph | 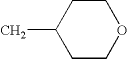 | |
| 1004 | H | Ph | 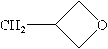 | |
| 1005 | H | Ph | 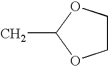 | 113-114 |
| 1006 | H | Ph | 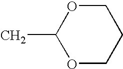 | |
| 1007 | H | Ph | 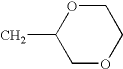 | |
| 1008 | H | Ph | 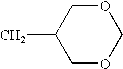 | |
| 1009 | H | Ph | 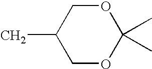 | |
| 1010 | H | Ph | 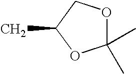 | oil |
| 1011 | H | Ph | 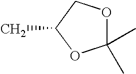 | oil |
| 1012 | H | Ph | 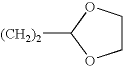 | |
| 1013 | H | Ph | 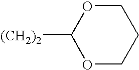 | |
| 1014 | H | Ph | allyl | 64-65 |
| 1015 | H | Ph | crotyl | |
| 1016 | H | Ph | 2-penten-1-yl | |
| 1017 | H | Ph | 2-methylallyl | |
| 1018 | H | Ph | 3-methyl-2-buten-1-yl | |
| 1019 | H | Ph | 2-chloro-2-propen-1-yl | |
| 1020 | H | Ph | 2-fluoro-2-propen-1-yl | |
| 1021 | H | Ph | 2-bromo-2-propen-1-yl | |
| 1022 | H | Ph | 3-chloro-2-buten-1-yl | |
| 1023 | H | Ph | 3-chloro-2-propen-1-yl | |
| 1024 | H | Ph | 3-bromo-2-propen-1-yl | |
| 1025 | H | Ph | 2,3-dichloro-2-propen-1-yl | |
| 1026 | H | Ph | cinnamyl | |
| 1027 | H | Ph | propargyl | 139-141 |
| 1028 | H | Ph | 2-butyn-1-yl | 141 |
| 1029 | H | Ph | phenylpropargyl | |
| 1030 | H | Ph | trimethylsilylpropargyl | |
| 1031 | H | Ph | CH₂CN | |
| 1032 | H | Ph | (CH₂)₂CN | |
| 1033 | H | 2-Th | CH₂COOEt | 179 |
| 1034 | H | 2-Th | CH₂COOPr | 168 |
| 1035 | H | 2-Th | CH₂CH₂COOiPr | 141 |
| 1036 | H | 2-Th | CH₂COOBu | |
| 1037 | H | 2-Th | CH₂COOiBu | |
| 1038 | H | 2-Th | CH₂COOsBu | |
| 1039 | H | 2-Th | CH₂COOtBu | 134 |
| 1040 | H | 2-Th | CH₂COOH | solid |
| 1041 | H | 2-Th | CH(Me)COOMe | 132 |
| 1042 | H | 2-Th | CH(Me)COOEt | 149 |
| 1043 | H | 2-Th | (CH₂)₂COOMe | |
| 1044 | H | 2-Th | (CH₂)₂COOEt | |
| 1045 | H | 2-Th | (CH₂)₃COOMe | |
| 1046 | H | 2-Th | (CH₂)₃COOEt | |
| 1047 | H | 2-Th | (CH₂)₃COOH | |
| 1048 | H | 2-Th | CH₂CONMe₂ | 227-228 |
| 1049 | 6-Me | 2-Th | CH₂CONMe₂ | >250 |
| 1050 | 6-Cl | 2-Th | CH₂CONMe₂ | >250 |
| 1051 | 7-Me | 2-Th | CH₂CONMe₂ | >250 |
| 1052 | 8-Me | 2-Th | CH₂CONMe₂ | 196 |
| 1053 | 6,7-Me₂ | 2-Th | CH₂CONMe₂ | >250 |
| 1054 | H | 2-Th | CH₂CONEt₂ | 170-171 |
| 1055 | 6,7-Me₂ | 2-Th | CH₂CONEt₂ | 216 |
| 1056 | H | 2-Th | CH₂CONPr₂ | |
| 1057 | H | 2-Th | CH₂CONiPr₂ | |
| 1058 | H | 2-Th | CH₂CONMe₂ | |
| 1059 | H | 2-Th | 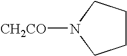 | |
| 1060 | H | 2-Th | 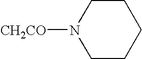 | |
| 1061 | H | 2-Th | 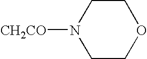 | 254 |
| 1062 | H | 2-Th | 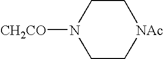 | |
| 1063 | H | 2-Th | CH₂CONMe(OMe) | 198-199 |
| 1064 | H | 2-Th | CH₂CONMePh | |
| 1065 | H | 2-Th | CH₂CONHMe | |
| 1066 | H | 2-Th | CH₂CONHEt | |
| 1067 | H | 2-Th | CH₂CONHPr | |
| 1068 | H | 2-Th | CH₂CONHiPr | |
| 1069 | H | 2-Th | CH₂CONHBu | |
| 1070 | H | 2-Th | CH₂CONHiBu | |
| 1071 | H | 2-Th | CH₂CONHsBu | |
| 1072 | H | 2-Th | CH₂CONHiBu | |
| 1073 | H | 2-Th | CH₂CONHPh | >250 |
| 1074 | H | 2-Th | CH₂COMe | |
| 1075 | H | 2-Th | CH(CH₃)COMe | 107 |
| 1076 | H | 2-Th | CH₂COCF₃ | |
| 1077 | H | 2-Th | CH₂COEt | |
| 1078 | H | 2-Th | CH₂COPr | |
| 1079 | H | 2-Th | CH₂COiPr | |
| 1080 | H | 2-Th | CH₂COtBu | |
| 1081 | H | 2-Th | CH₂OMe | 142 |
| 1082 | H | 2-Th | CH₂OEt | 110 |
| 1083 | H | 2-Th | (CH₂)₂OMe | 115 |
| 1084 | H | 2-Th | (CH₂)₂OEt | 82-83 |
| 1085 | H | 2-Th | (CH₂)₂OPr | |
| 1086 | H | 2-Th | (CH₂)₂OiPr | |
| 1087 | H | 2-Th | (CH₂)₂OtBu | oil |
| 1088 | H | 2-Th | (CH₂)₂O(CH₂)₂OMe | oil |
| 1089 | H | 2-Th | (CH₂)₂O(CH₂)₂OEt | |
| 1090 | H | 2-Th | (CH₂)₂OCH₂CF₃ | |
| 1091 | H | 2-Th | (CH₂)₂OPh | |
| 1092 | H | 2-Th | (CH₂)₃OH | |
| 1093 | H | 2-Th | (CH₂)₃OMe | |
| 1094 | H | 2-Th | (CH₂)₃OEt | |
| 1095 | H | 2-Th | (CH₂)₃OPr | |
| 1096 | H | 2-Th | (CH₂)₃OiPr | |
| 1097 | H | 2-Th | (CH₂)₃OtBu | |
| 1098 | H | 2-Th | (CH₂)₂CH(OMe)₂ | |
| 1100 | H | 2-Th | (CH₂)₂CH(OEt)₂ | |
| 1101 | H | 2-Th | CH₂CH(OMe)₂ | |
| 1102 | H | 2-Th | CH₂CH(OEt)₂ | 80 |
| 1103 | H | 2-Th | CH₂CH(OMe)CH₂OMe | oil |
| 1104 | H | 2-Th | CH₂SMe | 156 |
| 1105 | H | 2-Th | (CH₂)₂SMe | 111-112 |
| 1106 | H | 2-Th | (CH₂)₂SEt | oil |
| 1107 | H | 2-Th | (CH₂)₂SPr | |
| 1108 | H | 2-Th | (CH₂)₂SiPr | oil |
| 1109 | H | 2-Th | (CH₂)₂StBu | |
| 1110 | H | 2-Th | (CH₂)₂SPh | oil |
| 1111 | H | 2-Th | (CH₂)₂SCH₂CF₃ | oil |
| 1112 | H | 2-Th | (CH₂)₂S-cyclopentyl | oil |
| 1113 | H | 2-Th | 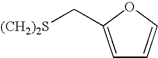 | oil |
| 1114 | H | 2-Th | (CH₂)₂S(O)Me | 183 |
| 1115 | H | 2-Th | (CH₂)₂S(O)₂Me | 219-220 |
| 1116 | H | 2-Th | (CH₂)₂S(O)Et | |
| 1117 | H | 2-Th | (CH₂)₂S(O)₂Et | |
| 1118 | H | 2-Th | (CH₂)₂S(O)Ph | |
| 1119 | H | 2-Th | (CH₂)₂S(O)₂Ph | 231 |
| 1120 | H | 2-Th | (CH₂)₂NH₂.HCl | >250 |
| 1121 | H | 2-Th | (CH₂)₂NHMe | |
| 1122 | H | 2-Th | (CH₂)₂NHEt | |
| 1123 | H | 2-Th | (CH₂)₂NHPr | |
| 1124 | H | 2-Th | (CH₂)₂NHiPr | |
| 1125 | H | 2-Th | (CH₂)₃NH₂ | |
| 1126 | H | 2-Th | (CH₂)₂NMe₂ | 95-96 |
| 1127 | H | 2-Th | (CH₂)₂NMe₂.HCl | |
| 1128 | H | 2-Th | (CH₂)₂NPr₂ | |
| 1129 | H | 2-Th | (CH₂)₂NiPr₂ | oil |
| 1130 | H | 2-Th | (CH₂)₂NiPr₂.HCl | 256-257 |
| 1131 | H | 2-Th | 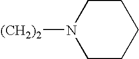 | 102-103 |
| 1132 | 6,7-Me₂ | 2-Th | 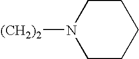 | 135 |
| 1133 | 6-Cl | 2-Th | 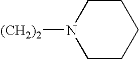 | 119 |
| 1134 | 7-Me | 2-Th | 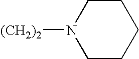 | oil |
| 1135 | H | 2-Th | 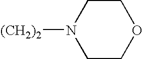 | 137 |
| 1136 | H | 2-Th | 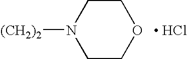 | >220 |
| 1137 | H | 2-Th | 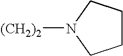 | 100-101 |
| 1138 | | 2-Th | 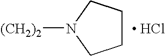 | 269-270 |
| 1139 | H | 2-Th | (CH₂)₃NMe₂ | 195-196 |
| 1140 | H | 2-Th | (CH₂)₃NMe₂.HCl | 206 |
| 1141 | H | 2-Th | (CH₂)₃NEt₂ | 85 |
| 1142 | H | 2-Th | (CH₂)₃NEt₂.HCl | |
| 1143 | H | 2-Th | 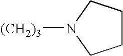 | |
| 1144 | H | 2-Th | 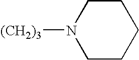 | |
| 1145 | H | 2-Th | 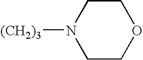 | oil |
| 1146 | H | 2-Th | CH₂CH(CH₃)CH₂NMe₂ | oil |
| 1147 | H | 2-Th | CH₂CH(CH₃)NMe₂ | oil |
| 1148 | H | 2-Th | CH₂CH(CH₃)NMe₂.HCl | 127-128 |
| 1149 | H | 2-Th | 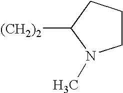 | oil |
| 1150 | H | 2-Th | 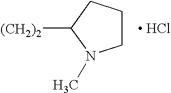 | 247-248 |
| 1151 | H | 2-Th | 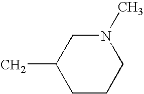 | oil |
| 1152 | H | 2-Th | 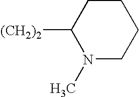 | oil |
| 1153 | H | 2-Th | 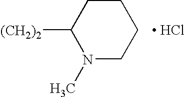 | 230-231 |
| 1154 | H | 2-Th | (CH₂)₂NHAc | |
| 1155 | H | 2-Th | (CH₂)₂NHCOEt | |
| 1156 | H | 2-Th | (CH₂)₂NHCOPr | |
| 1157 | H | 2-Th | (CH₂)₂NHCOiPr | |
| 1158 | H | 2-Th | (CH₂)₂NHCOBu | |
| 1159 | H | 2-Th | (CH₂)₂NHCOtBu | |
| 1160 | H | 2-Th | (CH₂)₂NHCOPh | |
| 1161 | H | 2-Th | (CH₂)₂NHCOOMe | |
| 1162 | H | 2-Th | (CH₂)₂NHCOOEt | |
| 1163 | H | 2-Th | (CH₂)₂NHCOOPr | |
| 1164 | H | 2-Th | (CH₂)₂NHCOOiPr | |
| 1165 | H | 2-Th | (CH₂)₂NHCOOtBu | 156-157 |
| 1166 | H | 2-Th | (CH₂)₂NHCONHMe | |
| 1167 | H | 2-Th | (CH₂)₂NHCONMe₂ | |
| 1168 | H | 2-Th | (CH₂)₂NHCONHEt | |
| 1169 | H | 2-Th | (CH₂)₂NHCONEt₂ | |
| 1170 | H | 2-Th | 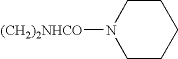 | |
| 1171 | H | 2-Th | 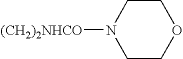 | |
| 1172 | H | 2-Th | 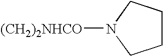 | |
| 1173 | H | 2-Th | (CH₂)₂NMeCONMe₂ | |
| 1174 | H | 2-Th | (CH₂)₂NMeCONEt₂ | |
| 1175 | H | 2-Th | (CH₂)₂NHSO₂Me | 236-237 |
| 1176 | H | 2-Th | (CH₂)₂NMeSO₂Me | |
| 1177 | H | 2-Th | (CH₂)₂NHSO₂Ph | |
| 1178 | H | 2-Th | (CH₂)₂NMeSO₂Ph | |
| 1179 | H | 2-Th | (CH₂)₂OH | 177 |
| 1180 | H | 2-Th | (CH₂)₂OAc | 125-126 |
| 1181 | H | 2-Th | (CH₂)₂COOPr | |
| 1182 | H | 2-Th | (CH₂)₂COOiPr | |
| 1183 | H | 2-Th | (CH₂)₂OCOBu | |
| 1184 | H | 2-Th | (CH₂)₂OCOtBu | |
| 1185 | H | 2-Th | (CH₂)₂COOCMe | |
| 1186 | H | 2-Th | (CH₂)₂COOCEt | |
| 1187 | H | 2-Th | (CH₂)₂OCOOPr | |
| 1188 | H | 2-Th | (CH₂)₂COOCiPr | |
| 1189 | H | 2-Th | (CH₂)₂COOCBu | |
| 1190 | H | 2-Th | (CH₂)₂COOCtBu | |
| 1191 | H | 2-Th | (CH₂)₂COONMe₂ | |
| 1192 | H | 2-Th | (CH₂)₂OCONEt₂ | |
| 1193 | H | 2-Th | 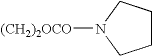 | |
| 1194 | H | 2-Th | 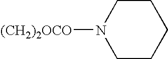 | |
| 1195 | H | 2-Th | 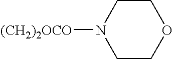 | |
| 1196 | H | 2-Th | CH₂CH(OH)Me | oil |
| 1197 | H | 2-Th | CH₂CH(OH)CH₂OH | oil |
| 1198 | H | 2-Th | Et | 148 |
| 1199 | H | 2-Th | Pr | 111 |
| 1200 | H | 2-Th | iPr | |
| 1201 | H | 2-Th | Bu | |
| 1202 | H | 2-Th | iBu | |
| 1203 | H | 2-Th | sBu | |
| 1204 | H | 2-Th | tBu | |
| 1205 | H | 2-Th | CH₂CH₂F | 184 |
| 1206 | H | 2-Th | CH₂CHF₂ | 181 |
| 1207 | H | 2-Th | CH₂CF₃ | 195-196 |
| 1208 | H | 2-Th | (CH₂)₂OF3 | 144 |
| 1209 | H | 2-Th | CH₂-cyclopropyl | 130-131 |
| 1210 | H | 2-Th | CH₂-cyclobutyl | 110 |
| 1211 | H | 2-Th | CH₂-cyclopentyl | |
| 1212 | H | 2-Th | CH₂-cyclohexyl | |
| 1213 | H | 2-Th | benzyl | |
| 1214 | H | 2-Th | 2-furfuryl | |
| 1215 | H | 2-Th | 3-furfuryl | |
| 1216 | H | 2-Th | 2-thienylmethyl | |
| 1217 | H | 2-Th | 3-thienylmethyl | |
| 1218 | H | 2-Th | 2-(5-chlorothienyl)methyl | 198 |
| 1219 | H | 2-Th | 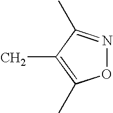 | |
| 1220 | H | 2-Th | 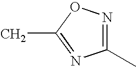 | 185 |
| 1221 | H | 2-Th | 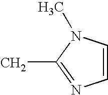 | 212 |
| 1222 | H | 2-Th | 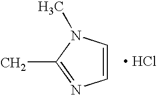 | >220 |
| 1223 | H | 2-Th | 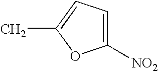 | |
| 1224 | H | 2-Th | 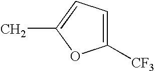 | |
| 1225 | H | 2-Th | 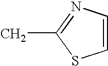 | |
| 1226 | H | 2-Th | 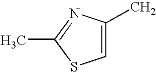 | |
| 1227 | H | 2-Th | 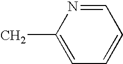 | |
| 1228 | H | 2-Th | 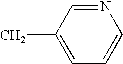 | 169 |
| 1229 | H | 2-Th | 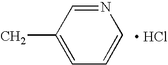 | >230 |
| 1230 | H | 2-Th | 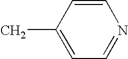 | |
| 1231 | H | 2-Th | 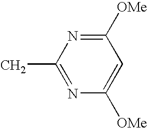 | |
| 1232 | H | 2-Th | 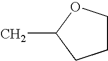 | 123 |
| 1233 | H | 2-Th | 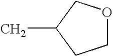 | 100 |
| 1234 | H | 2-Th | 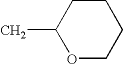 | 141-142 |
| 1235 | H | 2-Th | 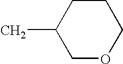 | |
| 1236 | H | 2-Th | 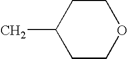 | |
| 1237 | H | 2-Th | 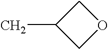 | |
| 1238 | H | 2-Th | 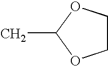 | 171 |
| 1239 | H | 2-Th | 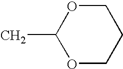 | |
| 1240 | H | 2-Th | 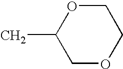 | |
| 1241 | H | 2-Th | 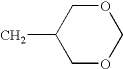 | |
| 1242 | H | 2-Th | 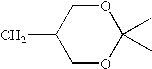 | |
| 1243 | H | 2-Th | 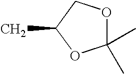 | oil |
| 1244 | H | 2-Th | 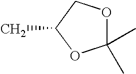 | oil |
| 1245 | H | 2-Th | 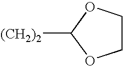 | 149 |
| 1246 | H | 2-Th | 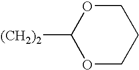 | |
| 1247 | H | 2-Th | aIlyl | 152 |
| 1248 | H | 2-Th | crotyl | 122 |
| 1249 | H | 2-Th | 2-penten-1-yl | |
| 1250 | H | 2-Th | 2-methylallyl | 126 |
| 1251 | H | 2-Th | 3-methyl-2-buten-1-yl | 129-130 |
| 1252 | H | 2-Th | 2-chloro-2-propen-1-yl | 130-131 |
| 1253 | H | 2-Th | 2-fluoro-2-propen-1-yl | |
| 1254 | H | 2-Th | 2-bromo-2-propen-1-yl | |
| 1255 | H | 2-Th | 3-chloro-2-buten-1-yl | 131-132 |
| 1256 | H | 2-Th | 3-chloro-2-propen-1-yl | |
| 1257 | H | 2-Th | 3-bromo-2-propen-1-yl | |
| 1258 | H | 2-Th | 2,3-dichloro-2-propen-1-yl | |
| 1259 | H | 2-Th | 3,3-dichloro-2-propen-1-yl | 60 |
| 1260 | H | 2-Th | cinnamyl | 189 |
| 1261 | H | 2-Th | propargyl | 222 |
| 1262 | H | 2-Th | 2-butyn-1-yl | 208 |
| 1263 | H | 2-Th | phenylpropargyl | |
| 1264 | H | 2-Th | trimethylsilylpropargyl | |
| 1265 | H | 2-Th | CH₂CN | 215 |
| 1266 | H | 2-Th | (CH₂)₂CN | |
| 1267 | H | iPr | allyl | |
| 1268 | H | iPr | crotyl | oil |
| 1269 | H | Ph | CH₂CONH₂ | >260 |
| 1270 | H | Th | CH₂CONH₂ | |
| 1271 | H | 5-Me-3-furyl | Me | 208 |
| 1272 | H | 5-Me-3-furyl | (CH₂)₂NEt₂ | 186 |
| 1273 | H | 5-Me-3-furyl | (CH₂)₂NEt₂.HCl | amorph |
| 1274 | H | 4-biphenylyl | Me | 154 |
| 1274a | H | 4-biphenylyl | (CH₂)₂NEt₂ | |
| 1275 | H | 4-biphenylyl | (CH₂)₂NEt₂.HCl | 187 |
| 1276 | H | 2-pyridyl | (CH₂)₂NEt₂.HCl | 230 |
| 1277 | H | cyclohexyl | (CH₂)₂NEt₂.HCl | 116 |
| 1278 | H | 6-Me-2-pyridyl | (CH₂)₂NEt₂.HCl | 223 |
| 1279 | H | m-tolyl | (CH₂)₂NEt₂.HCl | 201 |
| 1280 | H | 3,5-dichloro-2-thiazolyl | (CH₂)₂NEt₂ | 120 |
| 1281 | H | 3,5-dichloro-2-thiazolyl | (CH₂)₂NEt₂.HCl | >250 |
| 1282 | H | indan-5-yl | Me | 143 |
| 1283 | H | indan-5-yl | (CH₂)₂NEt₂ | oil |
| 1284 | H | indan-5-yl | (CH₂)₂NEt₂.HCl | 186 |
| 1285 | H | 2,5-di-Me-3-thienyl | (CH₂)₂NEt₂.HCl | 194 |
| 1286 | H | 4-isopropylphenyl | Me | 76 |
| 1287 | H | 4-isopropylphenyl | (CH₂)₂NEt₂ | oil |
| 1288 | H | 4-isopropylphenyl | (CH₂)₂NEt₂.HCl | 160 |
| 1289 | H | 2,3-dichiorophenyl | (CH₂)₂NEt₂.HCl | 217 |
| 1290 | H | 2,3-dimethylphenyl | (CH₂)₂NEt₂.HCl | amorph |
| 1291 | H | 2-furyl | (CH₂)₂NEt₂.HCl | 240 |
| 1292 | H | Ph | (CH₂)₂NH₂.HCl | 256 |
| 1293 | H | 2-Th | (CH₂)₂NH₂.HCl | >250 |
| 1294 | H | Ph | 1 ,3-dioxolan-4-ylmethyl | 79 |
| 1295 | H | 2-Th | 1 ,3-dioxolan-4-ylmethyl | 153 |
| 1296 | H | Ph | 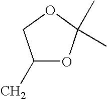 | oil |
| 1297 | H | 2-Th | 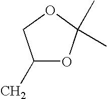 | oil |
| 1298 | H | Ph | 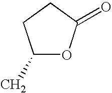 | oil |
| 1299 | H | 2-Th | 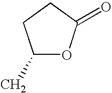 | oil |
| 1300 | H | Ph | 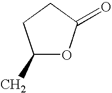 | oil |
| 1301 | H | 2-Th | 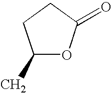 | oil |
| 1302 | 6,7-Me₂ | Ph | (CH₂)₂NEt₂.HCl | 245 |
| 1303 | H | Ph | 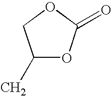 | 88 |
| 1304 | H | 2-Th | 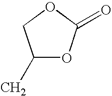 | 184 |
| 1305 | H | Ph | 3,5-dimethylpyrazol-1-yl | 123 |
| 1306 | H | 2-Th | 3,5-dimethylpyrazol-1-yl | 158 |
| 1307 | H | 1,2,3,4-tetrahydro-6- | (CH₂)₂NEt₂ | |
| | | naphthyl | | |
| 1308 | 6,7-Cl₂ | isopropyl | (CH₂)₂NEt₂ | oil |
| 1309 | H | 2,3,5-Cl₃-Ph | (CH₂)₂NEt₂ | oil |
| 1310 | H | 4-phenoxyphenyl | (CH₂)₂NEt₂ | oil |
| 1311 | H | Ph | 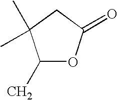 | 202 |
| 1312 | H | 2-Th | 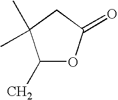 | 193 |
| 1313 | 6,7-Cl₂ | Ph | (CH₂)₂NEt₂ | 109 |
| 1314 | H | 4-SCH3-Ph | (CH₂)₂NEt₂ | oil |
| 1315 | H | Ph | 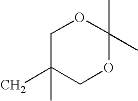 | 127 |
| 1316 | H | 2-Th | 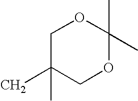 | 166 |
| 1317 | H | Ph | 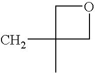 | 90 |
| 1318 | H | 2-Th | 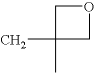 | 159 |
| 1319 | H | Ph | CH₂C(CH₃)₂OCH₃ | 102 |
| 1320 | H | 2-Th | CH₂C(CH₃)₂OCH₃ | 129 |
| 1321 | 6-CF₃ | 2-Th | isopropyl | 146 |
| 1322 | H | 4-cyclohexyl-Ph | (CH₂)₂NEt₂ | oil |
| 1323 | H | 4-cyclohexyl-Ph | (CH₂)₂NEt₂.HCl | 182 |
| 1324 | H | Ph | CH₂CH(CH₃)OCH₃ | 91 |
| 1325 | H | 2-Th | CH₂CH(CH₃)OCH₃ | 99 |
| 1326 | H | Ph | 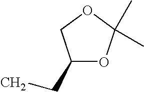 | oil |
| 1327 | H | 2-Th | 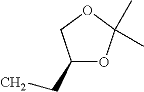 | 77 |
| 1328 | H | Ph | 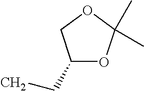 | oil |
| 1329 | H | 2-Th | 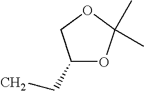 | 100 |
| 1330 | H | Ph | CH₂CH(CH₃)CH₂OEt | oil |
| 1331 | H | 2-Th | CH₂CH(CH₃)CH₂OEt | oil |
| 1332 | H | 4-Et-Ph | (CH₂)₂NEt₂ | oil |
| 1333 | H | Ph | CH₂C(CH₃)₂CH₂OMe | oil |
| 1334 | H | 2-Th | CH₂C(CH₃)₂CH₂OMe | |
| 1335 | H | 4-benzyloxy-Ph | (CH₂)₂NEt₂ | oil |
| 1336 | H | Ph | CH₂CH(CH₃)CH₂OMe | |
| 1337 | H | 2-Th | CH₂CH(CH₃)CH₂OMe | 104 |
| 1338 | H | 4-NO₂-Ph | Me | 246 |
| 1339 | H | 4-NO₂-Ph | (CH₂)₂NEt₂ | 112 |
| 1340 | H | Ph | 1-chlorocyclopropyl-1- | |
| | | | ylmethyl | |
| 1341 | H | 2-Th | 1-chlorocyclopropyl-1- | 180 |
| | | | ylmethyl | |
| 1342 | H | 4-OMe-Ph | 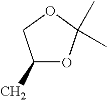 | oil |
| 1343 | H | 4-Me-Ph | 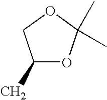 | oil |
| 1344 | H | Ph | NH₂ | 125 |
| 1345 | H | 2-Th | NH₂ | 164 |
| 1346 | H | Ph | 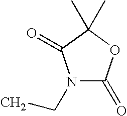 | 105 |
| 1347 | H | 2-Th | 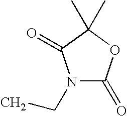 | 210 |
| 1348 | H | Ph | 2-(4-fluorophenoxy)ethyl | 111 |
| 1349 | H | 2-Th | 2-(4-fluorophenoxy)ethyl | 174 |
| 1350 | H | Ph | 2,2-dimethyl-1,3-dioxolan- | 157 |
| | | | 5-yl | |
| 1351 | H | 2-Th | 2,2-dimethyl-1,3-dioxolan- | 211 |
| | | | 5-yl | |
| 1352 | H | Ph | 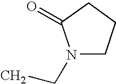 | 140 |
| 1353 | H | 2-Th | 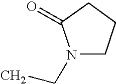 | 166 |
| 1354 | H | Ph | 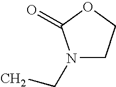 | |
| 1355 | H | 2-Th | 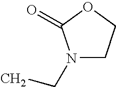 | oil |
| 1356 | H | 4-fluoro-3-methylphenyl | H | 244 |
| 1357 | H | 4-fluoro-3-methylphenyl | Me | 123 |
| 1358 | H | 4-fluoro-3-methylphenyl | (CH₂)₂NEt₂ | oil |
| 1359 | H | 4-fluoro-3-methylphenyl | (CH₂)₂NEt₂.HCl | 169 |
| 1360 | H | 4-fluoro-3-methylphenyl | 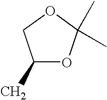 | |
| 1361 | 6,7-Me₂ | 4-fluoro-3-methylphenyl | (CH₂)₂NEt₂ | 66 |
| 1362 | 6,7-Me₂ | 4-fluoro-3-methylphenyl | (CH₂)₂NEt₂.HCl | 262 |
| 1363 | 6,7-Me₂ | 4-fluoro-3-methylphenyl | Me | 159 |
| 1364 | 6,7-Me₂ | 4-fluoro-3-methylphenyl | 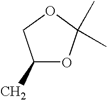 | 117 |
| 1365 | 6,7-Me₂ | 4-fluoro-3-methylphenyl | H | 264 |
| 1366 | 6,7-Me₂ | m-tolyl | Me | 150 |
| 1367 | 6,7-Me₂ | m-tolyl | 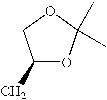 | 93 |
| 1368 | 6,7-Me₂ | m-tolyl | (CH₂)₂NEt₂ | 97 |
| 1369 | 6,7-Me₂ | m-tolyl | (CH₂)₂NEt₂.HCl | 252 |
| 1370 | 6,7-Me₂ | m-tolyl | H | >280 |
| 1371 | 7-Me | Ph | (CH₂)₂NEt₂.HCl | 228 |
| 1372 | H | Ph | 2-(allyloxy)ethyl | 44 |
| 1373 | H | 2-Th | 2-(allyloxy)ethyl | 95 |
| 1374 | 6,7-Me₂ | 3,5-dimethylphenyl | H | 288 |
| 1375 | 6,7-Me₂ | 3,5-dimethylphenyl | Me | 164 |
| 1376 | 6,7-Me₂ | 3,5-dimethylphenyl | (CH₂)₂NEt₂ | 107 |
| 1377 | 6,7-Me₂ | 3,5-dimethylphenyl | (CH₂)₂NEt₂HCl | 265 |
| 1378 | 6,7-Me₂ | 3,5-dimethylphenyl | 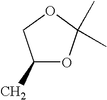 | 129 |
| 1379 | H | 3,5-dimethylphenyl | (CH₂)₂NEt₂.HCl | 209 |
| 1380 | H | 3,5-dimethylphenyl | 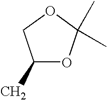 | 108 |
| 1381 | H | 2-Th | 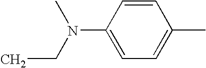 | oil |
| 1382 | H | Ph | 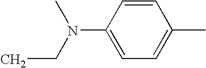 | oil |
| 1383 | H | Ph | 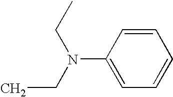 | 95 |
| 1384 | H | 2-Th | 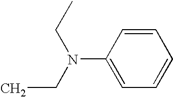 | 92 |
| 1385 | H | Ph | 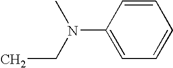 | oil |
| 1386 | H | 2-Th | 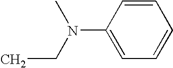 | oil |
| 1387 | 6,7-Me₂ | Ph | 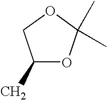 | 113 |
| 1388 | 6,7-Me₂ | 2-Th | 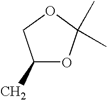 | 128 |
| 1389 | 6,7-Me₂ | Ph | (CH₂)₂NMe₂ | 197 |
| 1390 | 6,7-Me₂ | Ph | (CH₂)₂NMe₂.HCl | 267 |
| 1391 | 6,7-Me₂ | 2-Th | (CH₂)₂NMe₂ | 144 |
| 1392 | 6,7-Me₂ | 2-Th | (CH₂)₂NMe₂.HCl | 289 |
| 1393 | 7-Me | 2-Th | (CH₂)₂NEt₂.HCl | |
| 1394 | 6,7-Me₂ | 2-Th | (CH₂)₂NEt₂.HCl | |
| 1395 | 6,7-Me₂ | 2-Th | CH₂COOEt | oil |
| 1396 | 6,7-Me₂ | 2-Th | CH₂COOH | 263 |
| 1397 | H | 4-biphenylyl | H | >260 |
| 1398 | H | 5-methyl-2-furyl | H | >250 |
| 1399 | H | 3,5-dimethylphenyl | H | 202 |
| 1400 | H | 4-isopropylphenyl | H | 181 |
| 1401 | H | 2,4-dichloro-5-thiazolyl | H >260 | |
| 1402 | H | Ph | (CH₂)₂N⁽⁺⁾Me₃I⁽⁻⁾ | |
| 1403 | H | 2-Th | (CH₂)₂N⁽⁺⁾Me₃I⁽⁻⁾ | |
| 1404 | H | Ph | (CH₂)₂N⁽⁺⁾Me₃EtI⁽⁻⁾ | |
| 1405 | H | 2-Th | (CH₂)₂N⁽⁺⁾Me₃EtI⁽⁻⁾ | |
| 1406 | H | Ph | (CH₂)₂N⁽⁺⁾Et₂MeI⁽⁻⁾ | 220 |
| 1407 | H | 2-Th | (CH₂)₂N⁽⁺⁾Et₂MeI⁽⁻⁾ | |
| 1408 | H | Ph | (CH₂)₂N⁽⁺⁾Et₃I⁽⁻⁾ | |
| 1409 | H | 2-Th | (CH₂)₂N⁽⁺⁾Et₃I⁽⁻⁾ | 196 |
| 1410 | H | Ph | (CH₂)₂ON═C(CH₃)₂ | oil |
| 1411 | H | 2-Th | (CH₂)₂ON═C(CH₃)₂ | oil |
| 1412 | H | 2-benzothienyl | Me | |
| 1413 | H | 2-benzothienyl | (CH₂)₂NMe₂ | resin |
| 1414 | H | 2-benzothienyl | (CH₂)₂NMe₂.HCl | 265 |
| 1415 | 6,7-Me₂ | Ph | CH₂OMe | 135 |
| 1416 | 6,7-Me₂ | Ph | CH₂OEt | |
| 1417 | 6,7-Me₂ | Ph | (CH₂)₂OMe | |
| 1418 | 6,7-Me₂ | Ph | (CH₂)₂OEt | |
| 1419 | 6,7-Me₂ | 2-Th | CH₂OMe | 175 |
| 1420 | 6,7-Me₂ | 2-Th | CH₂OEt | |
| 1421 | 6,7-Me₂ | 2-Th | (CH₂)₂OMe | |
| 1422 | 6,7-Me₂ | 2-Th | (CH₂)₂OEt | |

Further physical data for some compounds from table 1:

Characteristic data of nuclear-magnetic resonance spectra (¹H-NMR data, δ (ppm)):

Example No. 405 (CDCl₃) 1.08 (tr, 6H, CH₂CH₃); 2.59 (s, 3H, CH₃); 2.68 (q, 4H, CH₂CH₃); 2.75 (tr, 2H, NCH₂); 4.35 (tr, 2H, CH₂Het); 7.82 (d, 1H, quinoxaline-H)

Example No. 408 (CDCl₃) 1.08 (tr, 6H, CH₂CH₃); 1.31 (d, 6H, iPrCH3); 2.69 (q, 4H, CH₂CH₃); 2.75 (tr, 2H, NCH₂); 3.62 (sept, 1H, methyne-H); 4.35 (tr, 2H, CH₂Het); 7.83 (d, 1H, quinoxaline-H)

Example No. 412 (CDCl₃) 1.07 (tr, 6H, CH₂CH₃); 1.49 (s, 9H, C(CH₃)₃); 2.65 (q, 4H, CH₂CH₃); 2.75 (tr, 2H, NCH₂); 4.32 (tr, 2H, CH₂Het); 7.82 (d, 1H, quinoxalinone-H)

Example No. 415 (CDCl₃) 1.12 (tr, 6H, CH₂CH₃); 1.88-2.12 (m, 6H, cyclopentyl-H); 2.43-2.58 (m, 2H, cyclopentyl-H); 2.71 (q, 4H, CH₂CH₃); 2.81 (tr, 2H, NCH₂); 4.44 (tr, 2H, CH₂Het); 7.97 (d, 1H, quinoxalinone-H)

Example No. 416 (CDCl₃) 1.42 (tr, 6H, CH₂CH₃); 1.1-2.0 (m, 10H, cyclohexyl-H); 3.2-3.4 (m, 7H); 4.73 (tr, 2H, CH₂Het); 7.87 (d, 1H, quinoxalinone-H)

Example No. 418 (CDCl₃) 1.03 (tr, 6H, CH₂CH₃); 2.64 (q, 4H, CH₂CH₃); 2.79 (tr, 2H, NCH₂); 4.40 (tr, 2H, CH₂Het); 8.00 (d, 1H, quinoxalinone-H)

Example No. 421 (CDCl₃) 1.09 (tr, 6H, CH₂CH₃); 2.69 (q, 4H, CH₂CH₃); 2.77 (tr, 2H, NCH₂); 3.13 (m, 2H, CH₂Ph); 3.28 (m, 2H, CH₂CH₂Ph); 4.35 (tr, 2H, CH₂Het); 7.86 (d, 1H, quinoxalinone-H)

Example No. 431 (CDCl₃) 1.08 (tr, 6H, CH₃); 2.68 (q, 4H, CH₂CH₃); 2.80 (tr, 2H, NCH₂); 4.42 (tr, 2H, CH₂Het); 8.50 (m, 2H, phenyl-H)

Example No. 434 (CDCl₃) 1.10 (tr, 6H, CH₂CH₃); 2.48 (s, 3H, CH3); 2.69 (q, 4H, CH₂CH₃); 2.80 (tr, 2H, NCH₂); 4.40 (tr, 2H, CH₂Het); 7.75 (s, 1H, quinoxalinone-H); 8.30 (m, 2H, phenyl-H)

Example No. 451 (CDCl₃) 1.07 (tr, 6H, CH₂CH₃); 2.66 (q, 4H, CH₂CH₃); 2.81 (tr, 2H, NCH₂); 4.43 (tr, 2H, CH₂Het); 8.23 (s, 1H, quinoxalinone-H); 8.34 (m, 2H, phenyl-H)

Example No. 465 (CDCl₃) 1.10 (tr, 6H, CH₂CH₃); 2.70 (q, 4H, CH₂CH₃); 2.82 (tr, 2H, NCH₂); 3.98, 4.02 (2s, 6H, 2 OCH₃); 4.42 (tr, 2H, CH₂Het); 6.91, 7.39 (2s, 2H, quinoxalinone-H); 8.28 (m, 2H, phenyl-H)

Example No. 478: (CDCl₃) 1.07 (tr, 6H, CH₃); 2.65 (q, 4H, CH₂CH₃); 2.79 (tr, 2H, NCH₂); 4.42 (tr, 2H, CH₂Het); 8.48 (d, 1H, Th)

Example No. 481: (CDCl₃) 1.08 (tr, 6H, CH₂CH₃); 2.51 (s, 3H, CH₃); 2.68 (q, 4H, CH₂CH₃); 2.80 (tr, 2H, NCH₂); 4.38 (tr, 2H, CH₂Het); 8.47 (d, 1H, Th)

Example No. 482: (CDCl₃) 1.50 (tr, 6H, CH₂CH₃); 3.29 (m, 6H, 3CH₂); 4.99 (tr, 2H, CH₂Het); 8.42 (2d, 2H, thienyl-H, quinoxaline-H)

Example No. 486: (CDCl₃) 1.05 (tr, 6H, CH₃); 2.65 (q, 4H, CH₂CH₃); 2.80 (tr, 2H, NCH₂); 4.38 (tr, 2H, CH₂Het); 8.48 (d, 1H, Th)

Example No. 487: (CDCl₃) 1.02 (tr, 6H, CH₃); 2.65 (q, 4H, CH₂CH₃); 2.78 (tr, 2H, NCH₂); 4.38 (tr, 2H, CH₂Het); 8.48 (d, 1H, Th)

Example No. 498 (CDCl₃) 1.10 (tr, 6H, CH₂CH₃); 2.72 (q, 4H, CH₂CH₃); 2.87 (tr, 2H, NCH₂); 4.50 (tr, 2H, CH₂Het); 8.19 (s, 1H, quinoxalinone-H); 8.52 (m, 2H, phenyl-H)

Example No. 501: (CDCl₃) 1.10 (tr, 6H, CH₂CH₃); 2.36, 2.42 (2s, 6H, 2CH₃); 2.69 (q, 4H, CH₂CH₃); 2.80 (tr, 2H, NCH₂); 4.42 (tr, 2H, CH₂Het); 8.42 (d, 1H, Th)

Example No. 507 (CDCl₃) 1.03 (tr, 6H, CH₂CH₃); 2.62 (q, 4H, CH₂CH₃); 2.79 (tr, 2H, NCH₂); 4.36 (tr, 2H, CH₂Het); 7.58, 7.98 (2s, 2H, quinoxalinone-H); 8.48 (d, 1H, thiophene-H)

Example No. 525: (CDCl₃) 1.09 (tr, 6H, CH₃); 1.47 (tr, 3H, ester-CH₃); 2.68 (q, 4H, CH₂CH₃); 2.83 (tr, 2H, NCH₂); 4.47 (m, 4H, OCH₂, CH₂Het); 8.44 (d, 1H, Th)

Example No. 526 (CDCl₃) 1.09 (tr, 6H, CH₂CH₃); 2.42 (s, 3H, tolyl-CH₃); 2.69 (q, 4H, CH₂CH₃); 2.80 (tr, 2H, NCH₂); 4.41 (tr, 2H, CH₂Het); 7.93 (d, 1H, quinoxalinone-H); 8.23 (d, 2H, phenyl-H)

Example No. 527 (CDCl₃) 1.09 (tr, 6H, CH₂CH₃); 2.42 (s, 3H, tolyl-CH₃); 2.69 (q, 4H, CH₂CH₃); 2.80 (tr, 2H, NCH₂); 4.41 (tr, 2H, CH₂Het); 7.95 (d, 1H, quinoxalinone-H); 8.10 (d, 2H, phenyl-H)

Example No. 529 (CDCl₃) 1.09 (tr, 6H, CH₂CH₃); 1.36 (s, 9H, tert-butyl); 2.68 (q, 4H, CH₂CH₃); 2.80 (tr, 2H, NCH₂); 4.42 (tr, 2H, CH₂Het); 7.94 (d, 1H, quinoxalinone-H); 8.12 (d, 2H, phenyl-H)

Example No. 530 (CDCl₃) 1.08 (tr, 6H, CH₂CH₃); 2.68 (q, 4H, CH₂CH₃); 2.80 (tr, 2H, NCH₂); 4.42 (tr, 2H, CH₂Het); 7.94 (d, 1H, quinoxalinone-H); 8.35 (d, 2H, phenyl-H)

Example No. 536 (CDCl₃) 1.10 (tr, 6H, CH₂CH₃); 2.69 (q, 4H, CH₂CH₃); 2.81 (tr, 2H, NCH₂); 3.89 (s, 3H, OCH₃); 4.42 (tr, 2H, CH₂Het); 7.92 (d, 1H, quinoxalinone-H); 6.99, 8.38 (2d, 4H, phenyl-H)

Example No. 539 (CDCl₃) 1.10 (tr, 6H, CH₂CH₃); 2.69 (q, 4H, CH₂CH₃); 2.82 (tr, 2H, NCH₂); 4.42 (tr, 2H, CH₂Het); 7.96 (d, 1H, quinoxalinone-H); 7.74, 8.48 (2d, 4H, phenyl-H)

Example No. 543 (CDCl₃) 1.08 (tr, 6H, CH₂CH₃); 2.35, 2.38 (2s, 6H, dimethylphenyl-CH₃); 2.69 (q, 4H, CH₂CH₃); 2.83 (tr, 2H, NCH₂); 4.42 (tr, 2H, CH₂Het); 7.16 (s, 1H, phenyl-H); 7.96 (d, 1H, quinoxalinone-H);

Example No. 546a (CDCl₃) 1.09 (tr, 6H, CH₂CH₃); 2.34, 2.37 (2s, 6H, dimethylphenyl-CH₃); 2.67 (q, 4H, CH₂CH₃); 2.80 (tr, 2H, NCH₂); 4.42 (tr, 2H, CH₂Het); 7.94 (d, 1H, quinoxalinone-H); 8.07 (d, 1H, phenyl-H); 8.09 (s, 1H, phenyl-H);

Example No. 547 (CDCl₃) 1.09 (tr, 6H, CH₂CH₃); 2.40 (s, 6H, dimethylphenyl-CH₃); 2.69 (q, 4H, CH₂CH₃); 2.81 (tr, 2H, NCH₂); 4.42 (tr, 2H, CH₂Het); 7.11 (s, 1H, phenyl-H); 7.88 (s, 2H, phenyl-H); 7.93 (d, 1H, quinoxalinone-H);

Example No. 548 (CDCl₃) 1.07 (tr, 6H, CH₂CH₃); 2.07 (s, 6H, trimethylphenyl-CH₃); 2.33 (s, 3H, trimethylphenyl-CH₃); 2.68 (q, 4H, CH₂CH₃); 2.83 (tr, 2H, NCH₂); 4.42 (tr, 2H, CH₂Het); 6.94 (s, 2H, phenyl-H); 7.97 (d, 1H, quinoxalinone-H);

Example No. 549 (CDCl₃) 1.11 (tr, 6H, CH₂CH₃); 2.70 (q, 4H, CH₂CH₃); 2.81 (tr, 2H, NCH₂); 3.96, 4.01 (2s, 6H, 20CH₃); 4.42 (tr, 2H, CH₂Het); 6.97 (d, 1H, phenyl-H); 7.95 (d, 1H, quinoxalinone-H); 8.02 (s, 1H, phenyl-H); 8.17 (d, 1H, phenyl-H);

Example No. 560 (CDCl₃) 1.09 (tr, 6H, CH₂CH₃); 2.69 (q, 4H, CH₂CH₃); 2.80 (tr, 2H, NCH₂); 4.30 (m, 4H, OCH₂); 4.41 (tr, 2H, CH₂Het); 6.96 (d, 1H, phenyl-H); 7.93 (d, 1H, quinoxalinone-H); 7.95 (m, 2H, phenyl-H);

Example No. 561 (CDCl₃) 1.08 (tr, 6H, CH₂CH₃); 2.67 (q, 4H, CH₂CH₃); 2.80 (tr, 2H, NCH₂); 4.40 (tr, 2H, CH₂Het); 7.55 (d, 1H, phenyl-H); 7.93 (d, 1H, quinoxalinone-H); 7.95 (m, 2H, phenyl-H);

Example No. 562 (CDCl₃) 1.08 (tr, 6H, CH₂CH₃); 2.68 (q, 4H, CH₂CH₃); 2.82 (tr, 2H, NCH₂); 4.40 (tr, 2H, CH₂Het); 7.28 (s, 1H, phenyl-H); 7.93 (d, 1H, quinoxalinone-H);

Example No. 569: (CDCl₃) 1.10 (tr, 6H, CH₃); 2.69 (q, 4H, CH₃CH₂); 2.80 (tr, 2H, CH₂NEt₂); 4.43 80 (tr, 2H, CH₂Het); 8.90 (d, 1H, thienyl)

Example No. 570 (CDCl₃) 1.08 (tr, 6H, CH₂CH₃); 2.68 (q, 4H, CH₂CH₃); 2.81 (tr, 2H, NCH₂); 4.42 (tr, 2H, CH₂Het); 6.62 (dd, 1H, furyl-H); 7.72 (d, 1H, furyl-H); 7.93 (d, 1H, furyl-H); 8.02 (d, 1H, quinoxalinone-H);

Example No. 575a (CDCl₃) 1.23 (tr, 6H, CH₂CH₃); 3.31 (m, 6H, CH₂CH₃, NCH₂); 4.82 (tr, 2H, CH₂Het); 8.09, 8.18 (2 dd, 2H, pyridyl-H, quinoxalinone-H); 8.88 (dd, 1H, pyridyl-H);

Example No. 578 (CDCl₃) 1.08 (tr, 6H, CH₂CH₃); 2.35 (s, 3H, CH₃Pyr); 2.68 (q, 4H, CH₂CH₃); 2.81 (tr, 2H, NCH₂); 4.41 (tr, 2H, CH₂Het); 7.97 (d, 1H, quinoxalinone-H); 8.60 (d, 1H, pyridyl-H)

Example No. 579 (CDCl₃) 1.10 (tr, 6H, CH₂CH₃); 2.48 (s, 3H, CH₃Pyr); 2.68 (q, 4H, CH₂CH₃); 2.82 (tr, 2H, NCH₂); 4.41 (tr, 2H, CH₂Het); 7.21 (d, 1H, pyridyl-H); 8.09 (d, 1H, quinoxalinone-H); 8.10 (s, 1H, pyridyl-H); 8.71 (d, 1H, pyridyl-H)

Example No. 581a (CDCl₃) 1.43 (tr, 6H, CH₂CH₃); 2.71 (s, 3H, CH₃Pyr); 3.31 (m, 6H, CH₂CH₃, NCH₂); 4.83 (tr, 2H, CH₂Het); 7.31 (d, 1H, pyridyl-H); 7.90 (d, 1H, pyridyl-H); 8.07 (d, 1H, quinoxalinone-H);

Example No. 588a (CDCl₃) 1.09 (tr, 6H, CH₂CH₃); 2.68 (q, 4H, CH₂CH₃); 2.78 (s, 3H, CH₃Th); 2.82 (tr, 2H, NCH₂); 4.43 (tr, 2H, CH₂Het); 7.02 (d, thienyl-H); 7.92 (d, 1H, quinoxalinone-H);

Example No. 595 (CDCl₃) 1.09 (tr, 6H, CH₂CH₃); 2.45, 2.63 (2s, 6H, 2 CH₃Th); 2.70 (q, 4H, CH₂CH₃); 2.78 (tr, 2H, NCH₂); 4.43 (tr, 2H, CH₂Het); 7.29 (s, thienyl-H); 7.89 (d, 1H, quinoxalinone-H);

Example No. 612: (CDCl₃) 1.32 (d, 6H, 2CH₃); 3.61 (sept, 1H, methyne-H); 3.78 (s, 3H, OCH₃); 2.80 (tr, 2H, NCH₂); 5.05 (s, 2H, CH₂Het); 7.98 (d, 1H, quinoxaline-H)

Example No. 638: (CDCl₃) 3.80 (s, 3H, OCH₃); 5.12 (s, 2H, CH₂Het); 8.32 (m, 2H, phenyl-H)

Example No. 687: (CDCl₃) 3.79 (s, 3H, OCH₃); 5.15 (s, 2H, CH₂Het); 8.48 (d, 1H, thienyl-H)

Example No. 688: (CDCl₃) 2.76 (s, 3H, CH₃); 3.78 (s, 3H, OCH₃); 5.14 (s, 2H, CH₂Het); 8.47 (d, 1H, thienyl-H)

Example No. 689: (CDCl₃) 2.52 (s, 3H, CH₃); 3.80 (s, 3H, OCH₃); 5.12 (s, 2H, CH₂Het); 8.47 (d, 1H, thienyl-H)

Example No. 691: (CDCl₃) 2.62 (s, 3H, CH₃); 3.80 (s, 3H, OCH₃); 5.17 (s, 2H, CH₂Het); 8.34 (d, 1H, thienyl-H)

Example No. 708: (CDCl₃) 2.35, 2.41 (2s, 6H, 2CH₃); 3.78 (s, 3H, OCH₃); 5.12 (s, 2H, CH₂Het); 8.45 (d, 1H, thienyl-H)

Example No. 855: (CDCl₃) 3.49 (s, 3H, CH₃); 5.80 (s, 2H, CH₂); 8.30 (m, 2H, Ph)

Example No. 856: (CDCl₃) 1.21 (tr, 3H, CH₃); 3.71 (q, 2H, CH₂Et); 5.82 (s, 2H, CH₂Het); 8.28 (m, 2H, Ph)

Example No. 857: (CDCl₃) 3.37 (s, 3H, CH₃); 3.80 (tr, 2H, OCH₂); 4.53 (tr, 2H, CH₂Het); 8.30 (m, 2H, Ph)

Example No. 858: (CDCl₃) 1.15 (tr, 3H, CH₃); 3.51 (q, 2H, CH₂Et); 3.82 (s, 2H, OCH₂); 8.30 (m, 2H, Ph)

Example No. 861 (CDCl₃) 1.09 (s, 9H, C(CH₃)₃); 3.78 (tr, 3H, CH₂O); 4.49 (tr, 2H, CH₂Het); 7.92 (d, 1H, quinoxalinone-H); 8.30 (m, 2H, phenyl-H)

Example No. 862 (CDCl₃) 3.32 (s, 3H, OCH₃); 3.50, 3.65 (2m, 4H, OCH₂CH₂O); 3.90 (tr, 2H, OCH₂); 4.57 (tr, 2H, CH₂Het); 7.92 (d, 1H, quinoxalinone-H); 8.30 (m, 2H, phenyl-H)

Example No. 883: (CDCl₃) 3.30 (tr, 2H, SCH₂); 4.50 (tr, 2H, CH₂Het); 7.92 (d, 1H, quinoxaline-H) 8.47 (m, 2H, Ph)

Example No. 907: (CDCl₃) 1.31 (tr, 6H, 2CH₃); 2.29 (sext, 2H, CH2) 3.05-3.30 (m, 6H, 3CH₂N);): 4.41 (tr, 2H, CH₂Het); 7.95 (d, 1H, quinox.-H) 8.38 (m, 2H, Ph)

Example No. 913: (CDCl₃) 2.00 (quintet, 2H, CH₂CH₂CH₂); 2.50 (m, 6H, 3CH₂N) 3.71 (tr, 4H, CH₂O;): 4.42 (tr, 2H, CH₂Het); 7.95 (d, 1H, quinox.-H) 8.40 (m, 2H, Ph)

Example No. 969 (CDCl₃) 1.72 (d, 6H, 2 CH₃); 5.35 (broad s, 1H, methyne-H); 7.95 (d, 1H, quinoxalinone-H); 8.28 (m, 2H, phenyl-H)

Example No. 1010: (CDCl₃) 1.32, 1.42 (2s, 6H, 2CH₃); 3.91, 4.17 (2dd, 2H, OCH₂); 4.34, 4.68 (2dd, 2H, CH₂Het); 4.56 (m, 1H, methyne-H), 8.30 (m, 2H, phenyl-H)

Example No. 1011: (CDCl₃) 1.32, 1.42 (2s, 6H, 2CH₃); 3.91, 4.17 (2dd, 2H, OCH₂); 4.34, 4.68 (2dd, 2H, CH₂Het); 4.56 (m, 1H, methyne-H), 8.30 (m, 2H, phenyl-H)

Example No. 1040: (DMSO) 5.10 (s, 2H, CH₂Het); 8.39 (d, 1H, thienyl-H)

Example No. 1087 (CDCl₃) 1.09 (s, 9H, C(CH₃)₃); 3.78 (tr, 3H, CH₂O); 4.50 (tr, 2H, CH₂Het); 7.89 (d, 1H, quinoxalinone-H); 8.48 (dd, 1H, thienyl-H)

Example No. 1088 (CDCl₃) 3.32 (s, 3H, OCH₃); 3.48, 3.65 (2m, 4H, OCH₂CH₂O); 3.90 (tr, 2H, OCH₂); 4.58 (tr, 2H, CH₂Het); 7.88 (d, 1H, quinoxalinone-H); 8.48 (dd, 1H, thienyl-H)

Example No. 1103: (CDCl₃) 3.34, 3.42 (2s 6H, 2CH₃); 3.48, 3.68 (2dd, 2H, OCH₂); 3.88 (m, 1H, methyne-H); 4.53 (dd, 2H, CH₂Het); 8.48 (d, 1H, thienyl)

Example No. 1106: (CDCl₃) 1.34 (tr, 3H, CH₃); 2.74 (q, 2H, CH₂CH₃); 2.90 (tr, 2H, SCH₂CH₂); 4.52 (tr, 2H, CH₂Het); 8.48 (d, 1H, thienyl)

Example No. 1108: (CDCl₃) 1.35 (d, 6H, 2CH₃); 2.91 (tr, 2H, SCH₂); 3.13 (sept, 1H, methyne-H); 4.52 (tr, 2H, CH₂Het); 8.48 (d, 1H, thienyl)

Example No. 1110: (CDCl₃) 3.29 (tr, 2H, SCH₂); 4.50 (tr, 2H, CH₂Het); 7.88 (d, 1H, quinoxaline-H); 8.48 (d, 1H, thienyl)

Example No. 1111: (CDCl₃) 3.07 (tr, 2H, SCH₂); 3.29 (q, 2H, SCH₂CF₃); 4.58 (tr, 2H, CH₂Het); 8.48 (d, 1H, thienyl)

Example No. 1112: (CDCl₃) 1.50-1.70, 1.70-1.80, 2.05-2.15 (3m, 8H, cyclopentyl); 2.91 (tr, 2H, SCH₂); 3.31 (quint, 1H, methyne-H); 4.53 (tr, 2H, CH₂Het); 8.48 (d, 1H, thienyl)

Example No. 1113: (CDCl₃) 2.90 (tr, 2H, SCH₂); 3.89 (s, 2H, SCH₂furyl); 4.51 (tr, 2H, CH₂Het); 6.31 (s, 2H, furyl-H); 8.48 (d, 1H, thienyl)

Example No. 1129: (CDCl₃) 1.02 (d, 12H, 4CH₃) 2.79 (tr, 2H, NCH₂); 3.09 (sept, 2H, methyne-H); 4.32 (tr, 2H, CH₂Het); 8.48 (d, 1H, thienyl)

Example No. 1134: (CDCl₃) 1.48 (m, 2H, CH₂) 1.62 (m, 4H, 2CH₂); 2.50-2.95 (m, 6H, 3NCH₂); 4.49 (tr, 2H, CH₂Het); 8.48 (d, 1H, thienyl)

Example No. 1145: (CDCl₃) 2.20 (sext, 2H, CH₂) 2.42-2.55 (m, 6H, 3CH₂N); 3.70 (tr, 4H, OCH₂); 4.44 (tr, 2H, CH₂Het); 7.92 (d, 1H, quinox.-H) 8.47 (d, 1H, thienyl)

Example No. 1146: (DMSO) 0.87 (d, 3H, CH₃) 2.12 (s, 6H, CH₃N); 2.10-2.40 (m, 3H, methyne-H, NCH₂); 4.33 (m, 2H, CH₂Het); 7.92 (d, 2H, quinox.-H) 8.40 (d, 1H, thienyl)

Example No. 1147: (CDCl₃) 2.33 (d, 3H, CH₃) 2.92 (s, 6H, CH₃N); 3.90 (m, 1H, methyne-H); 4.68, 4.95 (2dd, 2H, CH₂Het); 7.92 (d, 2H, quinox.-H) 8.45 (d, 1H, thienyl)

Example No. 1149: (CDCl₃) 1.65-1.90 (m, 6H, 3CH₂) 2.05-2.20 (m, 2H, CH₂N); 2.38 (s, 3H, CH₂); 3.12 (tr, 1H, methyne-H); 4.32, 4.49 (2m, 2H, CH₂Het); 7.92 (d, 2H, quinox.-H) 8.48 (d, 1H, thienyl)

Example No. 1151: (CDCl₃) 1.43 (m, 1H); 1.85-2.10 (m, 3H); 2.38 (s, 3H, CH₂); 2.55-2.90 (m, 3H); 3.78 (s, 3H, CH₃); 3.60 (dd, 2H); 4.32 (dd, 2H, CH₂Het); 7.92 (d, 2H, quinox.-H) 8.42 (d, 1H, thienyl)

Example No. 1152: (CDCl₃) 2.87 (s, 3H, CH₃); 3.60 (dd, 2H); 4.32, 4.60 (2m, 2H, CH₂Het); 7.92 (d, 2H, quinox.-H) 8.45 (d, 1H, thienyl)

Example No. 1196: (CDCl₃) 1.41 (d, 3H, CH₃); 4.30-4.40 (m, 2H); 4.45-4.60 (m, 1H); 7.92 (d, 2H, quinox.-H) 8.47 (d, 1H, thienyl)

Example No. 1197: (DMSO) 3.51 (m, 2H, OCH₂); 3.98 (m, 1H, methyne-H); 4.39 (d, 2H, CH₂Het); 4.80 (tr, 1H, CH₂OH), 4.93 (d, 1H, CHOH) 8.40 (d, 1H, thienyl).

Example No. 1243: (CDCl₃) 1.32, 1.41 (2s, 6H, 2CH₃); 3.92, 4.18 (2dd, 2H, OCH₂); 4.35, 4.70 (2dd, 2H, CH₂Het); 4.58 (m, 1H, methyne-H), 8.48 (d, 1H, thienyl)

Example No. 1244: (CDCl₃) 1.32, 1.41 (2s, 6H, 2CH₃); 3.92, 4.18 (2dd, 2H, OCH₂); 4.35, 4.70 (2dd, 2H, CH₂Het); 4.58 (m, 1H, methyne-H), 8.48 (d, 1H, thienyl)

Example No. 1268: (CDCl₃) 1.31 (d, 6H, iPrCH₃); 1.69 (d, 2H, crotyl-CH₃); 3.67 (sept, 1H, methyne-H); 4.83 (m, 2H, CH₂Het); 5.59, 5.82 (2m, 2H, olefin-H); 7.93 (d, 2H, quinox.-H)

Example No. 1273: (CDCl₃) 1.52 (tr, 6H, CH₂CH₃); 2.52 (s, 3H CH₃); 3.49 (m, 6H, CH₂CH₃, NCH₂); 5.03 (tr, 2H, CH₂Het); 6.32, 7.97 (2d, 2H, furyl-H); 8.32 (d, 1H, quinoxalinone-H);

Example No. 1282: (CDCl₃) 1.08 (tr, 6H, CH₂CH₃); 2.10 (m, 2H, 2-indanyl-CH₂); 2.48 (s, 3H CH₃); 2.80 (tr, 4H, CH₂CH₃); 2.96 (m, 4H, 1,3-indanyl-CH₂); 4.42 (tr, 2H, CH₂Het); 7.92 (d, 1H, quinoxalinone-H); 8.08 (d, 1H, indan-6-yl-H); 8.13 (d, 1H, indan-4-yl-H)

Example No. 1283: (CDCl₃) 1.08 (tr, 6H, CH₂CH₃); 2.10 (m, 2H, 2-indanyl-CH₂); 2.68 (q, 4H, CH₂CH₃), 2.80 (tr, 2H, NCH₂); 2.96 (m, 4H, 1,3-indanyl-CH₂); 4.42 (tr, 2H, CH₂Het); 7.92 (d, 1H, quinoxalinone-H); 8.08 (d, 1H, indan-6-yl-H); 8.13 (d, 1H, indan-4-yl-H)

Example No. 1287: (CDCl₃) 1.08 (tr, 6H, CH₂CH₃); 1.38 (d, 6H, isopropyl-CH₃); 2.68 (q, 4H, CH₂CH₃), 2.80 (tr, 4H, CH₂CH₃); 2.97 (sept, 1H, methyne-H); 4.42 (tr, 2H, CH₂Het); 7.33, 8.22 (2d, 4H, phenyl-H); 7.92 (d, 1H, quinoxalinone-H);

Example No. 1290: (CDCl₃) 1.52 (tr, 6H, CH₂CH₃); 2.34, 2.38 (2s, 6H, 2CH₃) 3.29 (m, 6H, CH₂CH₃, NCH₂); 5.00 (tr, 2H, CH₂Het); 8.0-8.2 (m, 5H, phenyl-H, quinoxalinone-H);

Example No. 1296: (CDCl₃) 1.32, 1.42 (2d, 6H, CH₃); 3.91, 4.18, 4.33, 4.69 (4dd, 4H, 2CH₂); 4.57 (quintet, 1H, methyne-H); 7.95 (d, 1H, quinoxalinone-H); 8.30 (m, 2H, phenyl-H)

Example No. 1297: (CDCl₃) 1.32, 1.42 (2d, 6H, CH₃); 3.91, 4.18, 4.33, 4.69 (4dd, 4H, 2CH₂); 4.57 (quintet, 1H, methyne-H); 7.90 (d, 1H, quinoxalinone-H); 8.48 (d, 1H, thienyl-H)

Example No. 1298: (CDCl₃) 2.2, 2.43-2.70 (2m, 1H, 3H CH₂CH₂CO); 4.43, 4.78 (2dd, 2H, CH₂N); 4.97 (m, 1H, methyne-H); 7.96 (d, 1H, quinoxalinone-H); 8.30 (m, 2H, phenyl-H)

Example No. 1299: (CDCl₃) 2.2, 2.43-2.70 (2m, 1H, 3H CH₂CH₂CO); 4.43, 4.78 (2dd, 2H, CH₂N); 4.97 (m, 1H, methyne-H); 7.90 (d, 1H, quinoxalinone-H); 8.45 (d, 1H, quinoxalinone-H)

Example No. 1300: (CDCl₃) 2.2, 2.43-2.70 (2m, 1H, 3H CH₂CH₂CO); 4.43, 4.78 (2dd, 2H, CH₂N); 4.97 (m, 1H, methyne-H); 7.96 (d, 1H, quinoxalinone-H); 8.30 (m, 2H, phenyl-H)

Example No. 1290: (CDCl₃) 1.52 (tr, 6H, CH₂CH₃); 2.34, 2.38 (2s, 6H, 2CH₃) 3.29 (m, 6H, CH₂CH₃, NCH₂); 5.00 (tr, 2H, CH₂Het); 8.0-8.2 (m, 5H, phenyl-H, quinoxalinone-H);

Example No. 1301: (CDCl₃) 2.2, 2.43-2.70 (2m, 1H, 3H CH₂CH₂CO); 4.43, 4.78 (2dd, 2H, CH₂N); 4.97 (m, 1H, methyne-H); 7.90 (d, 1H, quinoxalinone-H); 8.45 (d, 1H, quinoxalinone-H)

Example No. 1307: (CDCl₃) 1.08 (tr, 6H, CH₂CH₃); 1.80 (m, 4H, 2,3-bismethylene); 2.68 (q, 4H, CH₂CH₃); 2.77-2.90 (m, 4H, 1,4-bismethylene) 4.41 (tr, 2H, CH₂Het); 7.92 (2, 1H, quinoxalinone-H); 8.00 (s, 1H, tetralin-H); 8.02 (d, 1H, tetralin-H)

Example No. 1308: (CDCl₃) 1.02 (tr, 6H, CH₂CH₃); 1.29 (d, 6H, CH(CH₃)₂); 2.61 (q, 4H, CH₂CH₃); 2.74 (tr, 2H, CH₂N); 3.58 (sept, 1H, methyne-H); 4.25 (tr, 2H, CH₂Het); 7.50, 7.95 (2s, 2H, quinoxalinone-H);

Example No. 1309: (CDCl₃) 1.07 (tr, 6H, CH₂CH₃); 2.65 (q, 4H, CH₂CH₃); 2.80 (tr, 2H, CH₂N); 4.40 (tr, 2H, CH₂Het); 7.40, 7.57 (2d 2H, phenyl-H); 7.93 (d, 1H, quinoxalinone-H)

Example No. 1310: (CDCl₃) 1.09 (tr, 6H, CH₂CH₃); 2.68 (q, 4H, CH₂CH₃); 2.81 (tr, 2H, CH₂N); 4.42 (tr, 2H, CH₂Het); 7.40, 7.57 (2d 2H, phenyl-H); 7.92 (d, 1H, quinoxalinone-H); 8.36 (d 2H, phenyl-H)

Example No. 1314: (CDCl₃) 1.08 (tr, 6H, CH₂CH₃); 2.53 (s, 3H, SCH₃); 2.68 (q, 4H, CH₂CH₃); 2.80 (tr, 2H, CH₂N); 4.42 (tr, 2H, CH₂Het); 7.93 (d, 1H, quinoxalinone-H); 8.33 (d, 2H, phenyl-H)

Example No. 1315: (CDCl₃) 1.07 (tr, 6H, CH₂CH₃); 2.68 (q, 4H, CH₂CH₃); 2.82 (tr, 2H, CH₂N); 4.41 (tr, 2H, CH₂Het); 7.93 (d, 1H, quinoxalinone-H)

Example No. 1322: (CDCl₃) 1.09 (tr, 6H, CH₂CH₃); 1.20-1.60, 1.70-1.95 (2m, 10H, cyclohexyl-CH₂): 2.59 (m, 1H, methyne-H); 2.68 (q, 4H, CH₂CH₃); 2.83 (tr, 2H, CH₂N); 4.42 (tr, 2H, CH₂Het); 7.95 (d, 1H, quinoxalinone-H); 8.24 (d, 2H, phenyl-H)

Example No. 1326: (CDCl₃) 1.38, 1.50 (2s, 6H, CH₃); 1.9-2.2 (m, 2H, CH₂); 3.63, 4.11 (2dd, 2H, CH₂O); 4.27 (m, 1H, methyne-H); 4.49 (tr, 2H, CH₂Het); 7.95 (d, 1H, quinoxalinone-H); 8.29 (d, 2H, phenyl-H)

Example No. 1328: (CDCl₃) 1.38, 1.50 (2s, 6H, CH₃); 1.9-2.2 (m, 2H, CH₂); 3.63, 4.11 (2dd, 2H, CH₂O); 4.27 (m, 1H, methyne-H); 4.49 (tr, 2H, CH₂Het); 7.95 (d, 1H, quinoxalinone-H); 8.29 (d, 2H, phenyl-H)

Example No. 1330: (CDCl₃) 1.04 (d, 3H, CH(CH₃)); 1.29 (tr, 3H, CH₂CH₃); 2.42 (m, 1H, methyne-H), 3.38 (d, 2H, CHCH₂O); 3.43 (q, 2H, OCH₂CH₃), 4.37 (d, 2H, CH₂Het); 7.93 (d, 1H, quinoxalinone-H); 8.29 (d, 2H, phenyl-H)

Example No. 1331: (CDCl₃) 1.05 (d, 3H, CH(CH₃)); 1.28 (tr, 3H, CH₂CH₃); 2.42 (m, 1H, methyne-H), 3.37 (d, 2H, CHCH₂O); 3.43 (q, 2H, OCH₂CH₃), 4.38 (d, 2H, CH₂Het); 7.90 (d, 1H, quinoxalinone-H); 8.48 (d, 1H, thienyl-H)

Example No. 1332: (CDCl₃) 1.08 (tr, 6H, NCH₂CH₃); 1.25 (tr, 6H, C₆H₄CH₂CH₃); 2.68 (q, 6H, NCH₂CH₃, C₆H₄CH₂CH₃); 2.79 (tr, 2H, CH₂N); 4.41 (tr, 2H, CH₂Het); 7.93 (d, 1H, quinoxalinone-H), 8.23 (d, 2H, phenyl-H)

Example No. 1333: (CDCl₃) 1.03 (s, 6H, C(CH₃)₂); 3.10 (3, 2H, OCH₂); 3.35 (s, 3H, OCH₃); 4.39 (tr, 2H, CH₂Het); 7.92 (d, 1H, quinoxalinone-H); 8.28 (d, 2H, phenyl-H)

Example No. 1335: (CDCl₃) 1.09 (tr, 6H, CH₂CH₃); 2.68 (q, 4H, CH₂CH₃); 2.80 (tr, 2H, CH₂N); 4.42 (tr, 2H, CH₂Het); 5.15 (s, 2H, benzyl-CH₂); 7.08, 8.38 (2d, 4H, C₆H₄); 7.93 (d, 1H, quinoxalinone-H)

Example No. 1342: (CDCl₃) 1.32, 1.42 (2d, 6H, CH₃); 3.89 (s, 3H OCH₃); 3.91, 4.16, 4.33, 4.69 (4dd, 4H, 2CH₂); 4.57 (quintet, 1H, methyne-H); 7.00, 8.38 (2d, 4H, phenyl-H); 7.93 (d, 1H, quinoxalinone-H)

Example No. 1343: (CDCl₃) 1.32, 1.42 (2d, 6H, CH₃); 3.42 (s, 3H tolyl-CH₃); 3.91, 4.16, 4.33, 4.69 (4dd, 4H, 2CH₂); 4.57 (quintet, 1H, methyne-H); 7.28, 8.22 (2d, 4H, phenyl-H); 7.93 (d, 1H, quinoxalinone-H);

Example No. 1355: (CDCl₃) 3.68 (2tr, 4H, 2CH₂N); 4.29 (tr, 2H, CH₂Het); 4.58 (tr, 2H CH₂O); 7.93 (d, 1H, quinoxalinone-H); 8.45 (d. 1H, thienyl-H)

Example No. 1358: (CDCl₃) 1.09 (tr, 6H, CH₂CH₃); 2.36 (s, 3H, CH₃aryl); 2.68 (q, 4H, CH₂CH₃); 2.80 (tr, 2H, CH₂N); 4.42 (tr, 2H, CH₂Het); 5.15 (s, 2H, benzyl-CH₂); 7.93 (d, 1H, quinoxalinone-H); 8.20 (m, 2H, phenyl-H)

Example No. 1360: (CDCl₃) 1.32, 1.42 (2d, 6H, CH₃); 2.37 (s, 3H CH₃aryl); 3.91, 4.16, 4.33, 4.69 (4dd, 4H, 2CH₂); 4.57. (quintet, 1H, methyne-H); 7.92 (d, 1H, quinoxalinone-H); 8.20 (m, 2H, phenyl-H);

Example No. 1381: (CDCl₃) 2.25 (s, 3H, tolyl-CH₃); 2.92 (s, 3H, NCH₃); 3.73 (tr, 2H, CH₂N); 4.52 (tr, 2H, CH₂Het); 6.75, 7.09 (2d, 4H, phenyl-H); 7.91 (d, 1H, quinoxalinone-H); 8.49 (d, 1H, thienyl-H)

Example No. 1382: (CDCl₃) 2.27 (s, 3H, tolyl-CH₃); 2.97 (s, 3H, NCH₃); 3.75 (tr, 2H, CH₂N); 4.50 (tr, 2H, CH₂Het); 6.75, 7.09 (2d, 4H, phenyl-H); 7.93 (d, 1H, quinoxalinone-H); 8.29 (m, 2H, phenyl-H)

Example No. 1385: (CDCl₃) 3.00 (s, 3H, NCH₃); 3.78 (tr, 2H, CH₂N); 4.52 (tr, 2H, CH₂Het); 7.93 (d, 1H, quinoxalinone-H); 8.30 (m, 2H, phenyl-H)

Example No. 1386: (CDCl₃) 2.98 (s, 3H, NCH₃); 3.80 (tr, 2H, CH₂N); 4.57 (tr, 2H, CH₂Het); 7.91 (d, 1H, quinoxalinone-H); 8.49 (d, 1H, thienyl-H)

Example No. 1395: (CDCl₃) 1.28 (tr, 3H, CH₂CH₃); 2.36, 2.40 (2s, 6H, 6,7-Me₂); 4.25 (q, 2H, CH₂CH₃); 5.10 (s, 2H, CH₂N); 6.88, 7.69 (2s, 2H, quinoxalinone-H); 7.28, 7.55, 8.45 (tr, d, d, 3H, thienyl-H)

Example No. 1410: (CDCl₃) 1.69, 1.87 (2s, 6H, 2CH₃); 4.43, 4.63 (2tr, 4H, 2CH₂); 7.93 (d, 1H, quinoxalinone-H); 8.33 (m, 2H, phenyl-H)

Example No. 1411: (CDCl₃) 1.65, 1.85 (2s, 6H, 2CH₃); 4.44, 4.65 (2tr, 4H, 2CH₂); 7.89 (d, 1H, quinoxalinone-H); 8.49 (m, 2H, phenyl-H)

Example No. 1413: (CDCl₃) 1.09 (tr, 6H, 2CH₃); 2.70 (tr, 4H, 2CH₂CH₃); 2.83 (2H, tr, CH₂N); 4.49 (tr, 2H, CH₂Het); 7.91 (d, 1H, quinoxalinone-H); 8.03, 9.09, 9.22 (2d, s, 3H, benzothiophene-H)

TABLE 2
Compounds of the formula (I-2)

| | |
|---|---|
| | (I-2) |
| 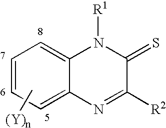 | |

| Ex. | Y | R¹ | R¹ | m.p. [° C.] |
|---|---|---|---|---|
| 2-1 | H | Ph | Me | 187-188 |
| 2-2 | H | 2-Th | Me | 113 |
| 2-3 | H | Ph | (CH₂)₂NEt₂ | |
| 2-4 | H | 2-Th | (CH₂)₂NEt₂ | |
| 2-5 | H | Ph | H | |
| 2-6 | 5-Me | Ph | H | |
| 2-7 | 6-Me | Ph | H | |
| 2-8 | 7-Me | Ph | H | |
| 2-9 | 8-Me | Ph | H | |
| 2-10 | 5-Cl | Ph | H | |
| 2-11 | 6-Cl | Ph | H | |
| 2-12 | 7-Cl | Ph | H | |
| 2-13 | 8-Cl | Ph | H | |
| 2-14 | 5-F | Ph | H | |
| 2-15 | 15 | Ph | H | |
| 2-16 | 16 | Ph | H | |
| 2-17 | 8-F | Ph | H | |
| 2-18 | 5-OMe | Ph | H | |
| 2-19 | 6-OMe | Ph | H | |
| 2-20 | 7-OMe | Ph | H | |
| 2-21 | 8-OMe | Ph | H | |
| 2-22 | 5-CF₃ | Ph | H | |
| 2-23 | 6-CF₃ | Ph | H | |
| 2-24 | 7-CF₃ | Ph | H | |
| 2-25 | 8-CF₃ | Ph | H | |
| 2-26 | 6,7-Me₂ | Ph | H | |
| 2-27 | 5,7-Me₂ | Ph | H | |
| 2-28 | 5,6-Me₂ | Ph | H | |
| 2-29 | 7,8-Me₂ | Ph | H | |
| 2-30 | 5,7-Me₂ | Ph | H | |
| 2-31 | 6,8-Me₂ | Ph | H | |
| 2-32 | 5,8-Me₂ | Ph | H | |
| 2-33 | 6,7-Cl₂ | Ph | H | |
| 2-34 | 5,6-Cl₂ | Ph | H | |
| 2-35 | 5,7-Cl₂ | Ph | H | |
| 2-36 | 7,8-Cl₂ | Ph | H | |
| 2-37 | 6,8-Cl₂ | Ph | H | |
| 2-38 | 5,8-Cl₂ | Ph | H | |
| 2-39 | 6,7-(OMe)₂ | Ph | H | |
| 2-40 | 6,7-O-CF₂-O- | Ph | H | |
| 2-41 | 6,7-F₂ | Ph | H | |
| 2-42 | 5,7-F₂ | Ph | H | |
| 2-43 | 5,6-F₂ | Ph | H | |
| 2-44 | 7,8-F₂ | Ph | H | |
| 2-45 | 6,8-F₂ | Ph | H | |
| 2-46 | 5,8-F₂ | Ph | H | |
| 2-47 | 6,7-(CF₃)₂ | Ph | H | |
| 2-48 | 5,7-(CF₃)₂ | Ph | H | |
| 2-49 | 5,6-(CF₃)₂ | Ph | H | |
| 2-50 | 7,8-(CF₃)₂ | Ph | H | |
| 2-51 | 6,8-(CF₃)₂ | Ph | H | |
| 2-52 | 5,8-(CF₃)₂ | Ph | H | |
| 2-53 | 6-Cl, 7-F | Ph | H | |
| 2-54 | H | 2-Th | H | |
| 2-55 | 5-Me | 2-Th | H | |
| 2-56 | 6-Me | 2-Th | H | |
| 2-57 | 7-Me | 2-Th | H | |
| 2-58 | 8-Me | 2-Th | H | |
| 2-59 | 5-Cl | 2-Th | H | |
| 2-60 | 6-Cl | 2-Th | H | |
| 2-61 | 7-Cl | 2-Th | H | |
| 2-62 | 8-Cl | 2-Th | H | |
| 2-63 | 5-F | 2-Th | H | |
| 2-64 | 6-F | 2-Th | H | |
| 2-65 | 7-F | 2-Th | H | |
| 2-66 | 8-F | 2-Th | H | |
| 2-67 | 5-OMe | 2-Th | H | |
| 2-68 | 6-OMe | 2-Th | H | |
| 2-69 | 7-OMe | 2-Th | H | |
| 2-70 | 8-OMe | 2-Th | H | |
| 2-71 | 5-CF₃ | 2-Th | H | |
| 2-72 | 6-CF₃ | 2-Th | H | |
| 2-73 | 7-CF₃ | 2-Th | H | |
| 2-74 | 8-CF₃ | 2-Th | H | |
| 2-75 | 6,7-Me₂ | 2-Th | H | |
| 2-76 | 5,7-Me₂ | 2-Th | H | |
| 2-77 | 5,6-Me₂ | 2-Th | H | |
| 2-78 | 7,8-Me₂ | 2-Th | H | |
| 2-79 | 5,7-Me₂ | 2-Th | H | |
| 2-80 | 6,8-Me₂ | 2-Th | H | |
| 2-81 | 5,8-Me₂ | 2-Th | H | |
| 2-82 | 6,7-Cl₂ | 2-Th | H | |
| 2-83 | 5,6-Cl₂ | 2-Th | H | |
| 2-84 | 5,7-Cl₂ | 2-Th | H | |
| 2-85 | 7,8-Cl₂ | 2-Th | H | |
| 2-86 | 6,8-Cl₂ | 2-Th | H | |
| 2-87 | 5,8-Cl₂ | 2-Th | H | |
| 2-88 | 6,7-(OMe)₂ | 2-Th | H | |
| 2-89 | 6,7-O-CF₂-O- | 2-Th | H | |
| 2-90 | 6,7-F₂ | 2-Th | H | |
| 2-91 | 5,7-F₂ | 2-Th | H | |
| 2-92 | 5,6-F₂ | 2-Th | H | |
| 2-93 | 7,8-F₂ | 2-Th | H | |
| 2-94 | 6,8-F₂ | 2-Th | H | |
| 2-95 | 5,8-F₂ | 2-Th | H | |
| 2-96 | 6,7-(CF₃)₂ | 2-Th | H | |
| 2-97 | 5,7-(CF₃)₂ | 2-Th | H | |
| 2-98 | 5,6-(CF₃)₂ | 2-Th | H | |
| 2-99 | 7,8-(CF₃)₂ | 2-Th | H | |
| 2-100 | 6,8-(CF₃)₂ | 2-Th | H | |
| 2-101 | 5,8-(CF₃)₂ | 2-Th | H | |
| 2-102 | 6-Cl, 7-F | 2-Th | H | |
| 2-103 | H | 2-Th | Me | |
| 2-104 | H | 2-Th | Me | |
| 2-105 | H | 2-Th | (CH₂)₂NEt₂ | |
| 2-106 | H | 2-Th | (CH₂)₂NEt₂ | |
| 2-107 | H | Ph | CH₂COOEt | |
| 2-108 | H | Ph | CH₂COOPr | |
| 2-109 | H | Ph | CH₂CH₂COOiPr | |
| 2-110 | H | Ph | CH₂COOBu | |
| 2-111 | H | Ph | CH₂COOiBu | |
| 2-112 | H | Ph | CH₂COOsBu | |
| 2-113 | H | Ph | CH₂COOtBu | |
| 2-114 | H | Ph | CH₂COOH | |
| 2-115 | H | Ph | CH(Me)COOMe | |
| 2-116 | H | Ph | CH(Me)COOEt | |
| 2-117 | H | Ph | (CH₂)₂COOMe | |
| 2-118 | H | Ph | (CH₂)₂COOEt | |
| 2-119 | H | Ph | (CH₂)₃COOMe | |
| 2-120 | H | Ph | (CH₂)₃COOEt | |
| 2-121 | H | Ph | (CH₂)₃COOH | |
| 2-122 | H | Ph | CH₂CONMe₂ | |
| 2-123 | H | Ph | CH₂CONEt₂ | |
| 2-124 | H | Ph | CH₂CONPr₂ | |
| 2-125 | H | Ph | CH₂CONiPr₂ | |
| 2-126 | H | Ph | CH₂CONMeEt | |
| 2-127 | H | Ph | 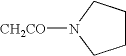 | |
| 2-128 | H | Ph | 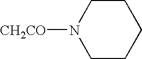 | |
| 2-129 | H | Ph | 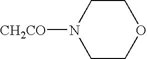 | |
| 2-130 | H | Ph | 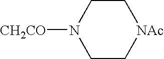 | |
| 2-131 | H | Ph | CH₂CONMe(OMe) | |
| 2-132 | H | Ph | CH₂CONMePh | |
| 2-133 | H | Ph | CH₂CONHMe | |
| 2-134 | H | Ph | CH₂CONHEt | |
| 2-135 | H | Ph | CH₂CONHPr | |
| 2-136 | H | Ph | CH₂CONHiPr | |
| 2-137 | H | Ph | CH₂CONHBu | |
| 2-138 | H | Ph | CH₂CONHiBu | |
| 2-139 | H | Ph | CH₂CONHsBu | |
| 2-140 | H | Ph | CH₂CONHtBu | |
| 2-141 | H | Ph | CH₂CONHPh | |
| 2-142 | H | Ph | CH₂COMe | |
| 2-143 | H | Ph | CH(CH₃)COMe | |
| 2-144 | H | Ph | CH₂COCF₃ | |
| 2-145 | H | Ph | CH₂COEt | |
| 2-146 | H | Ph | CH₂COPr | |
| 2-147 | H | Ph | CH₂COiPr | |
| 2-148 | H | Ph | CH₂COtBu | |
| 2-149 | H | Ph | CH₂OMe | |
| 2-150 | H | Ph | CH₂OEt | |
| 2-151 | H | Ph | (CH₂)₂OMe | |
| 2-152 | H | Ph | (CH₂)₂OE1 | |
| 2-153 | H | Ph | (CH₂)₂OPr | |
| 2-154 | H | Ph | (CH₂)₂OiPr | |
| 2-155 | H | Ph | (CH₂)₂OtBu | |
| 2-156 | H | Ph | (CH₂)₂O(CH₂)₂OMe | |
| 2-157 | H | Ph | (CH₂)₂O(CH₂)₂OEt | |
| 2-158 | H | Ph | (CH₂)₂COH₂CF₃ | |
| 2-159 | H | Ph | (CH₂)₂OPh | |
| 2-160 | H | Ph | (CH₂)₃CH | |
| 2-161 | H | Ph | (CH₂)₃OMe | |
| 2-162 | H | Ph | (CH₂)₃OEt | |
| 2-163 | H | Ph | (CH₂)₃OPr | |
| 2-164 | H | Ph | (CH₂)₃OiPr | |
| 2-165 | H | Ph | (CH₂)₃OtBu | |
| 2-166 | H | Ph | (CH₂)₂(OMe)₂ | |
| 2-167 | H | Ph | (CH₂)₂(OEt)₂ | |
| 2-168 | H | Ph | CH₂(OMe)₂ | |
| 2-169 | H | Ph | CH₂(OEt)₂ | |
| 2-170 | H | Ph | CH₂CH(OMe)CH₂OMe | |
| 2-171 | H | Ph | CH₂SMe | |
| 2-172 | H | Ph | (CH₂)₂SMe | |
| 2-173 | H | Ph | (CH₂)₂SEt | |
| 2-174 | H | Ph | (CH₂)₂SPr | |
| 2-175 | H | Ph | (CH₂)₂SiPr | |
| 2-176 | H | Ph | (CH₂)₂StBu | |
| 2-177 | H | Ph | (CH₂)₂SPh | |
| 2-178 | H | Ph | (CH₂)₂SCH₂CF₃ | |
| 2-179 | H | Ph | (CH₂)₂S-cyclopentyl | |
| 2-180 | H | Ph | 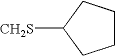 | |
| 2-181 | H | Ph | (CH₂)₂S(O)Me | |
| 2-182 | H | Ph | (CH₂)₂S(O)₂Me | |
| 2-183 | H | Ph | (CH₂)₂S(O)Et | |
| 2-184 | H | Ph | (CH₂)₂S(O)₂Et | |
| 2-185 | H | Ph | (CH₂)₂S(O)Ph | |
| 2-186 | H | Ph | (CH₂)₂S(O)₂Ph | |
| 2-187 | H | Ph | (CH₂)₂NH₂ | |
| 2-188 | H | Ph | (CH₂)₂NHMe • HCl | |
| 2-189 | H | Ph | (CH₂)₂NHEt | |
| 2-190 | H | Ph | (CH₂)₂NHPr | |
| 2-191 | H | Ph | (CH₂)₂NHiPr | |
| 2-192 | H | Ph | (CH₂)₃NH₂ | |
| 2-193 | H | Ph | (CH₂)₂NMe₂ | |
| 2-194 | H | Ph | (CH₂)₂NMe₂ • HCl | |
| 2-195 | H | Ph | (CH₂)₂NPr₂ | |
| 2-196 | H | Ph | (CH₂)₂NiPr₂ | |
| 2-197 | H | Ph | 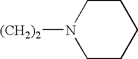 | |
| 2-198 | H | Ph | 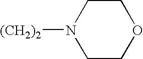 | |
| 2-199 | H | Ph | 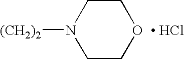 | |
| 2-200 | H | Ph | 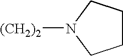 | |
| 2-201 | H | Ph | (CH₂)₃NMe₂ | |
| 2-202 | H | Ph | (CH₂)₃NMe₂ • HCl | |
| 2-203 | H | Ph | (CH₂)₃NEt₂ | |
| 2-204 | H | Ph | (CH₂)₃NEt₂ • HCl | |
| 2-205 | H | Ph | 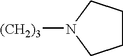 | |
| 2-206 | H | Ph | 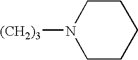 | |
| 2-207 | H | Ph | 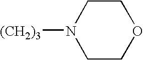 | |
| 2-208 | H | Ph | CH₂CH(CH₃)CH₂NMe₂ | |
| 2-209 | H | Ph | CH(CH₃)CH₂NMe₂ | |
| 2-210 | H | Ph | CH₂CH(CH₃)NMe₂ | |
| 2-211 | H | Ph | CH₂CH(CH₃)NMe₂ • HCl | |
| 2-212 | H | Ph | 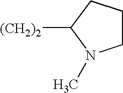 | |
| 2-213 | H | Ph | 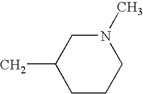 | |
| 2-214 | H | Ph | 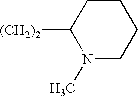 | |
| 2-215 | H | Ph | (CH₂)₂NHAc | |
| 2-216 | H | Ph | (CH₂)₂NHCOEt | |
| 2-217 | H | Ph | (CH₂)₂NHCOPr | |
| 2-218 | H | Ph | (CH₂)₂NHCOiPr | |
| 2-219 | H | Ph | (CH₂)₂NHCOBu | |
| 2-220 | H | Ph | (CH₂)₂NHCOtBu | |
| 2-221 | H | Ph | (CH₂)₂NHCOPh | |
| 2-222 | H | Ph | (CH₂)₂NHCOOMe | |
| 2-223 | H | Ph | (CH₂)₂NHCOOEt | |
| 2-224 | H | Ph | (CH₂)₂NHCOOPr | |
| 2-225 | H | Ph | (CH₂)₂NHCOOiPr | |
| 2-226 | H | Ph | (CH₂)₂NHCOOtBu | |
| 2-227 | 7-CF₃ | Ph | (CH₂)₂NHCOOtBu | |
| 2-228 | H | Ph | (CH₂)₂NHCONHMe | |
| 2-229 | H | Ph | (CH₂)₂NHCONMe₂ | |
| 2-230 | H | Ph | (CH₂)₂NHCONHEt | |
| 2-231 | H | Ph | (CH₂)₂NHCONEt₂ | |
| 2-232 | H | Ph | 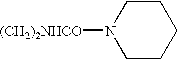 | |
| 2-233 | H | Ph | 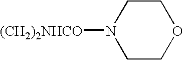 | |
| 2-234 | H | Ph | 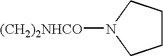 | |
| 2-235 | H | Ph | (CH₂)₂NMeCONMe₂ | |
| 2-236 | H | Ph | (CH₂)₂NMeCONEt₂ | |
| 2-237 | H | Ph | (CH₂)₂NHSO₂Me | |
| 2-238 | H | Ph | (CH₂)₂NMeSO₂Me | |
| 2-239 | H | Ph | (CH₂)₂NHSO₂Ph | |
| 2-240 | H | Ph | (CH₂)₂NMeSO₂Ph | |
| 2-241 | H | Ph | (CH₂)₂CH | |
| 2-242 | 7-NO₂ | Ph | (CH₂)₂CH | |
| 2-243 | H | Ph | (CH₂)₂OAc | |
| 2-244 | H | Ph | (CH₂)₂OCOPr | |
| 2-245 | H | Ph | (CH₂)₂OCOiPr | |
| 2-246 | H | Ph | (CH₂)₂OCOBu | |
| 2-247 | H | Ph | (CH₂)₂OCOtBu | |
| 2-248 | H | Ph | (CH₂)₂OCOOMe | |
| 2-249 | H | Ph | (CH₂)₂OCOOEt | |
| 2-250 | H | Ph | (CH₂)₂OCOOPr | |
| 2-251 | H | Ph | (CH₂)₂OCOOiPr | |
| 2-252 | H | Ph | (CH₂)₂OCOOBu | |
| 2-253 | H | Ph | (CH₂)₂OCOOtBu | |
| 2-254 | H | Ph | (CH₂)₂OCONMe₂ | |
| 2-255 | H | Ph | (CH₂)₂OCONEt₂ | |
| 2-256 | H | Ph | 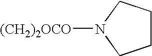 | |
| 2-257 | H | Ph | 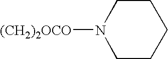 | |
| 2-258 | H | Ph | 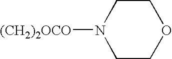 | |
| 2-259 | H | Ph | CH₂CH(OH)Me | |
| 2-260 | H | Ph | CH₂CH(OH)CH₂OH | |
| 2-261 | H | Ph | Et | |
| 2-262 | H | Ph | Pr | |
| 2-263 | H | Ph | iPr | |
| 2-264 | H | Ph | Bu | |
| 2-265 | H | Ph | iBu | |
| 2-266 | H | Ph | sBu | |
| 2-267 | H | Ph | tBu | |
| 2-268 | H | Ph | CH₂CH₂F | |
| 2-269 | H | Ph | CH₂CHF₂ | |
| 2-270 | H | Ph | CH₂CF₃ | |
| 2-271 | H | Ph | (CH₂)₂CF₃ | |
| 2-272 | H | Ph | CH₂-cyclopropyl | |
| 2-273 | H | Ph | CH₂-cyclobutyl | |
| 2-274 | H | Ph | CH₂-cyclopentyl | |
| 2-275 | H | Ph | CH₂-cyclohexyl | |
| 2-276 | H | Ph | benzyl | |
| 2-277 | H | Ph | 2-furfuryl | |
| 2-278 | H | Ph | 3-furfuryl | |
| 2-279 | H | Ph | 2-thienylmethyl | |
| 2-280 | H | Ph | 3-thienylmethyl | |
| 2-281 | H | Ph | 2-(5-chlorothienyl)- | |
| | | | methyl | |
| 2-282 | H | Ph | 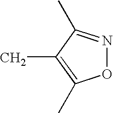 | |
| 2-283 | H | Ph | 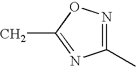 | |
| 2-284 | H | Ph | 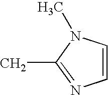 | |
| 2-285 | H | Ph | 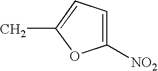 | |
| 2-286 | H | Ph | 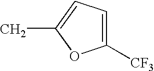 | |
| 2-287 | H | Ph | 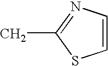 | |
| 2-288 | H | Ph | 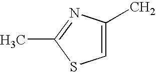 | |
| 2-289 | H | Ph | 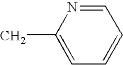 | |
| 2-290 | H | Ph | 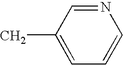 | |
| 2-291 | H | Ph | 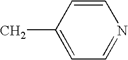 | |
| 2-292 | H | Ph | 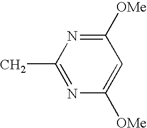 | |
| 2-293 | H | Ph | 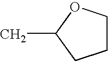 | |
| 2-294 | H | Ph | 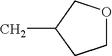 | |
| 2-295 | H | Ph | 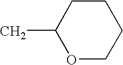 | |
| 2-296 | H | Ph | 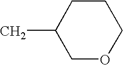 | |
| 2-297 | H | Ph | 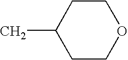 | |
| 2-298 | H | Ph | 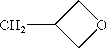 | |
| 2-299 | H | Ph | 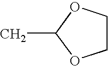 | |
| 2-300 | H | Ph | 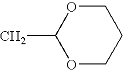 | |
| 2-301 | H | Ph | 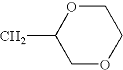 | |
| 2-302 | H | Ph | 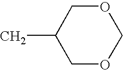 | |
| 2-303 | H | Ph | 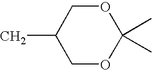 | |
| 2-304 | H | Ph | 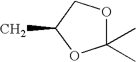 | |
| 2-305 | H | Ph | 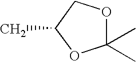 | |
| 2-306 | H | Ph | 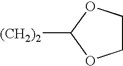 | |
| 2-307 | H | Ph | 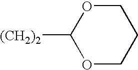 | |
| 2-308 | H | 2-Th | CH₂COOEt | |
| 2-309 | H | 2-Th | CH₂COOPr | |
| 2-310 | H | 2-Th | CH₂CH₂COOiPr | |
| 2-311 | H | 2-Th | CH₂COOBu | |
| 2-312 | H | 2-Th | CH₂COOiBu | |
| 2-313 | H | 2-Th | CH₂COOsBu | |
| 2-314 | H | 2-Th | CH₂COOtBu | |
| 2-315 | H | 2-Th | CH₂COOH | |
| 2-316 | H | 2-Th | CH(Me)COOMe | |
| 2-317 | H | 2-Th | CH(Me)COOEt | |
| 2-318 | H | 2-Th | (CH₂)₂COOMe | |
| 2-319 | H | 2-Th | (CH₂)₂COOEt | |
| 2-320 | H | 2-Th | (CH₂)₃COOMe | |
| 2-321 | H | 2-Th | (CH₂)₃COOEt | |
| 2-322 | H | 2-Th | (CH₂)₃COOH | |
| 2-323 | H | 2-Th | CH₂CONMe₂ | |
| 2-324 | H | 2-Th | CH₂CONEt₂ | |
| 2-325 | H | 2-Th | CH₂CONPr₂ | |
| 2-326 | H | 2-Th | CH₂CONiPr₂ | |
| 2-327 | H | 2-Th | CH₂CONMeEt | |
| 2-328 | H | 2-Th | 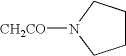 | |
| 2-329 | H | 2-Th | 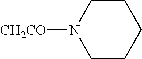 | |
| 2-330 | H | 2-Th | 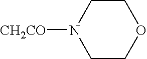 | |
| 2-331 | H | 2-Th | 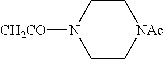 | |
| 2-332 | H | 2-Th | CH₂CONMe(OMe) | |
| 2-333 | H | 2-Th | CH₂CONMePh | |
| 2-334 | H | 2-Th | CH₂CONHMe | |
| 2-335 | H | 2-Th | CH₂CONHEt | |
| 2-336 | H | 2-Th | CH₂CONHPr | |
| 2-337 | H | 2-Th | CH₂CONHiPr | |
| 2-338 | H | 2-Th | CH₂CONHBu | |
| 2-339 | H | 2-Th | CH₂CONHiBu | |
| 2-340 | H | 2-Th | CH₂CONHsBu | |
| 2-341 | H | 2-Th | CH₂CONHtBu | |
| 2-342 | H | 2-Th | CH₂CONHPh | |
| 2-343 | H | 2-Th | CH₂COMe | |
| 2-344 | H | 2-Th | CH(CH₃)COMe | |
| 2-345 | H | 2-Th | CH₂COCF₃ | |
| 2-346 | H | 2-Th | CH₂COEt | |
| 2-347 | H | 2-Th | CH₂COPr | |
| 2-348 | H | 2-Th | CH₂COiPr | |
| 2-349 | H | 2-Th | CH₂COtBu | |
| 2-350 | H | 2-Th | CH₂OMe | |
| 2-351 | H | 2-Th | CH₂OEt | |
| 2-352 | H | 2-Th | (CH₂)₂OMe | |
| 2-353 | H | 2-Th | (CH₂)₂OEt | |
| 2-354 | H | 2-Th | (CH₂)₂OPr | |
| 2-355 | H | 2-Th | (CH₂)₂OiPr | |
| 2-356 | H | 2-Th | (CH₂)₂OtBu | |
| 2-357 | H | 2-Th | (CH₂)₂O(CH₂)₂OMe | |
| 2-358 | H | 2-Th | (CH₂)₂O(CH₂)₂OEt | |
| 2-359 | H | 2-Th | (CH₂)₂COH₂CF₃ | |
| 2-360 | H | 2-Th | (CH₂)₂OPh | |
| 2-361 | H | 2-Th | (CH₂)₃OH | |
| 2-362 | H | 2-Th | (CH₂)₃OMe | |
| 2-363 | H | 2-Th | (CH₂)₃OEt | |
| 2-364 | H | 2-Th | (CH₂)₃OPr | |
| 2-365 | H | 2-Th | (CH₂)₃OiPr | |
| 2-366 | H | 2-Th | (CH₂)₃OtBu | |
| 2-367 | H | 2-Th | (CH₂)₂(OMe)₂ | |
| 2-368 | H | 2-Th | (CH₂)₂(OEt)₂ | |
| 2-369 | H | 2-Th | CH₂(OMe)₂ | |
| 2-370 | H | 2-Th | CH₂(OEt)₂ | |
| 2-371 | H | 2-Th | CH₂CH(OMe)CH₂OMe | |
| 2-372 | H | 2-Th | CH₂SMe | |
| 2-373 | H | 2-Th | (CH₂)₂SMe | |
| 2-374 | H | 2-Th | (CH₂)₂SEt | |
| 2-375 | H | 2-Th | (CH₂)₂SPr | |
| 2-376 | H | 2-Th | (CH₂)₂SiPr | |
| 2-377 | H | 2-Th | (CH₂)₂StBu | |
| 2-378 | H | 2-Th | (CH₂)₂SPh | |
| 2-379 | H | 2-Th | (CH₂)₂SCH₂CF₃ | |
| 2-380 | H | 2-Th | (CH₂)₂S-cyclopentyl | |
| 2-381 | H | 2-Th | 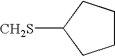 | |
| 2-382 | H | 2-Th | (CH₂)₂S(O)Me | |
| 2-383 | H | 2-Th | (CH₂)₂S(O)₂Me | |
| 2-384 | H | 2-Th | (CH₂)₂S(O)Et | |
| 2-385 | H | 2-Th | (CH₂)₂S(O)₂Et | |
| 2-386 | H | 2-Th | (CH₂)₂S(O)Ph | |
| 2-387 | H | 2-Th | (CH₂)₂S(O)₂Ph | |
| 2-388 | H | 2-Th | (CH₂)₂NH₂ | |
| 2-389 | H | 2-Th | (CH₂)₂NHMe • HCl | |
| 2-390 | H | 2-Th | (CH₂)₂NHEt | |
| 2-391 | H | 2-Th | (CH₂)₂NHPr | |
| 2-392 | H | 2-Th | (CH₂)₂NHiPr | |
| 2-393 | H | 2-Th | (CH₂)₃NH₂ | |
| 2-394 | H | 2-Th | (CH₂)₂NMe₂ | |
| 2-395 | H | 2-Th | (CH₂)₂NMe₂ | |
| 2-396 | H | 2-Th | (CH₂)₂NPr₂ | |
| 2-397 | H | 2-Th | (CH₂)₂NiPr₂ | |
| 2-398 | H | 2-Th | 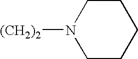 | |
| 2-399 | H | 2-Th | 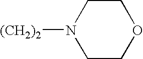 | |
| 2-400 | H | 2-Th | 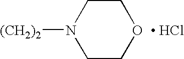 | |
| 2-401 | H | 2-Th | 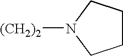 | |
| 2-402 | H | 2-Th | (CH₂)₃NMe₂ | |
| 2-403 | H | 2-Th | (CH₂)₃NMe₂ • HCl | |
| 2-404 | H | 2-Th | (CH₂)₃NEt₂ | |
| 2-405 | H | 2-Th | (CH₂)₃NEt₂ • HCl | |
| 2-406 | H | 2-Th | 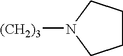 | |
| 2-407 | H | 2-Th | 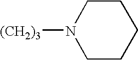 | |
| 2-408 | H | 2-Th | 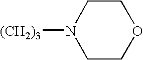 | |
| 2-409 | H | 2-Th | CH₂CH(CH₃)CH₂NMe₂ | |
| 2-410 | H | 2-Th | CH(CH_{3)CH}₂NMe₂ | |
| 2-411 | H | 2-Th | CH₂CH(CH₃)NMe₂ | |
| 2-412 | H | 2-Th | CH₂CH(CH₃)NMe₂ • HCl | |
| 2-413 | H | 2-Th | 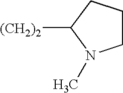 | |
| 2-414 | H | 2-Th | 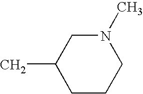 | |
| 2-415 | H | 2-Th | 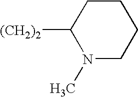 | |
| 2-416 | H | 2-Th | (CH₂)₂NHAc | |
| 2-417 | H | 2-Th | (CH₂)₂NHCOEt | |
| 2-418 | H | 2-Th | (CH₂)₂NHCOPr | |
| 2-419 | H | 2-Th | (CH₂)₂NHCOiPr | |
| 2-420 | H | 2-Th | (CH₂)₂NHCOBu | |
| 2-421 | H | 2-Th | (CH₂)₂NHCOtBu | |
| 2-422 | H | 2-Th | (CH₂)₂NHCOPh | |
| 2-423 | H | 2-Th | (CH₂)₂NHCOOMe | |
| 2-424 | H | 2-Th | (CH₂)₂NHOOOEt | |
| 2-425 | H | 2-Th | (CH₂)₂NHCOOPr | |
| 2-426 | H | 2-Th | (CH₂)₂NHCOOiPr | |
| 2-427 | H | 2-Th | (CH₂)₂NHCOOtBu | |
| 2-428 | 7-CF₃ | 2-Th | (CH₂)₂NHCOOtBu | |
| 2-429 | H | 2-Th | (CH₂)₂NHCONHMe | |
| 2-430 | H | 2-Th | (CH₂)₂NHCONMe₂ | |
| 2-431 | H | 2-Th | (CH₂)₂NHCONHEt | |
| 2-432 | H | 2-Th | (CH₂)₂NHCONEt₂ | |
| 2-433 | H | 2-Th | 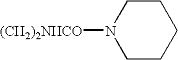 | |
| 2-434 | H | 2-Th | 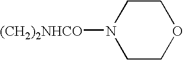 | |
| 2-435 | H | 2-Th | 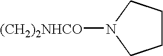 | |
| 2-436 | H | 2-Th | (CH₂)₂NMeCONMe₂ | |
| 2-437 | H | 2-Th | (CH₂)₂NMeCONEt₂ | |
| 2-438 | H | 2-Th | (CH₂)₂NHSO₂Me | |
| 2-439 | H | 2-Th | (CH₂)₂NMeSO₂Me | |
| 2-440 | H | 2-Th | (CH₂)₂NHSO₂Ph | |
| 2-441 | H | 2-Th | (CH₂)₂NMeSO₂Ph | |
| 2-442 | H | 2-Th | (CH₂)₂OH | |
| 2-443 | 7-NO₂ | 2-Th | (CH₂)₂OH | |
| 2-444 | H | 2-Th | (CH₂)₂OAc | |
| 2-445 | H | 2-Th | (CH₂)₂OCOPr | |
| 2-446 | H | 2-Th | (CH₂)₂OCOiPr | |
| 2-447 | H | 2-Th | (CH₂)₂OCOBu | |
| 2-448 | H | 2-Th | (CH₂)₂OCOtBu | |
| 2-449 | H | 2-Th | (CH₂)₂OCOOMe | |
| 2-450 | H | 2-Th | (CH₂)₂OCOOEt | |
| 2-451 | H | 2-Th | (CH₂)₂OCOOPr | |
| 2-452 | H | 2-Th | (CH₂)₂OCOOiPr | |
| 2-453 | H | 2-Th | (CH₂)₂OCOOBu | |
| 2-454 | H | 2-Th | (CH₂)₂OCOOtBu | |
| 2-455 | H | 2-Th | (CH₂)₂OCONMe₂ | |
| 2-456 | H | 2-Th | (CH₂)₂OCONEt₂ | |
| 2-457 | H | 2-Th | 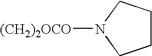 | |
| 2-458 | H | 2-Th | 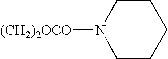 | |
| 2-459 | H | 2-Th | 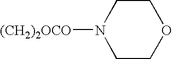 | |
| 2-460 | H | 2-Th | CH₂CH(OH)Me | |
| 2-461 | H | 2-Th | CH₂CH(OH)CH₂OH | |
| 2-462 | H | 2-Th | Et | |
| 2-463 | H | 2-Th | Pr | |
| 2-464 | H | 2-Th | iPr | |
| 2-465 | H | 2-Th | Bu | |
| 2-466 | H | 2-Th | iBu | |
| 2-467 | H | 2-Th | sBu | |
| 2-468 | H | 2-Th | tBu | |
| 2-469 | H | 2-Th | CH₂CH₂F | |
| 2-470 | H | 2-Th | CH₂CHF₂ | |
| 2-471 | H | 2-Th | CH₂CF₃ | |
| 2-472 | H | 2-Th | (CH₂)₂CF₃ | |
| 2-473 | H | 2-Th | CH₂-cyclopropyl | |
| 2-474 | H | 2-Th | CH₂-cyclobutyl | |
| 2-475 | H | 2-Th | CH₂-cyclopentyl | |
| 2-476 | H | 2-Th | CH₂-cyclohexyl | |
| 2-477 | H | 2-Th | benzyl | |
| 2-478 | H | 2-Th | 2-furfuryl | |
| 2-479 | H | 2-Th | 3-furfuryl | |
| 2-480 | H | 2-Th | 2-thienylmethyl | |
| 2-481 | H | 2-Th | 3-thienylmethyl | |
| 2-482 | H | 2-Th | 2-(5-chlorothienyl)- | |
| | | | methyl | |
| 2-483 | H | 2-Th | 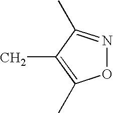 | |
| 2-484 | H | 2-Th | 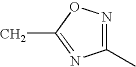 | |
| 2-485 | H | 2-Th | 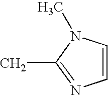 | |
| 2-486 | H | 2-Th | 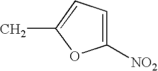 | |
| 2-487 | H | 2-Th | 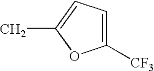 | |
| 2-488 | H | 2-Th | 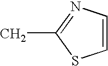 | |
| 2-489 | H | 2-Th | 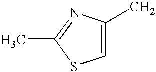 | |
| 2-490 | H | 2-Th | 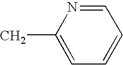 | |
| 2-491 | H | 2-Th | 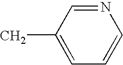 | |
| 2-492 | H | 2-Th | 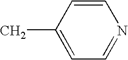 | |
| 2-493 | H | 2-Th | 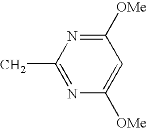 | |
| 2-494 | H | 2-Th | 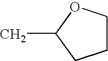 | |
| 2-495 | H | 2-Th | 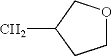 | |
| 2-496 | H | 2-Th | 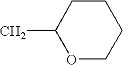 | |
| 2-497 | H | 2-Th | 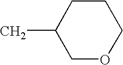 | |
| 2-498 | H | 2-Th | 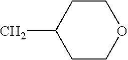 | |
| 2-499 | H | 2-Th | 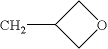 | |
| 2-500 | H | 2-Th | 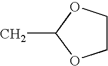 | |
| 2-501 | H | 2-Th | 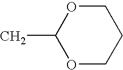 | |
| 2-502 | H | 2-Th | 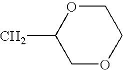 | |
| 2-503 | H | 2-Th | 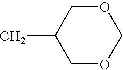 | |
| 2-504 | H | 2-Th | 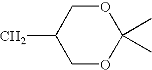 | |
| 2-505 | H | 2-Th | 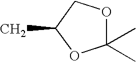 | |
| 2-506 | H | 2-Th | 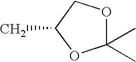 | |
| 2-507 | H | 2-Th | 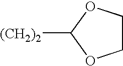 | |
| 2-508 | H | 2-Th | 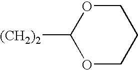 | |
| 2-509 | 5-Me | 2-Th | H | |
| 2-510 | 6-Me | 2-Th | H | |
| 2-511 | 7-Me | 2-Th | H | |
| 2-512 | 8-Me | 2-Th | H | |
| 2-513 | 5-Cl | 2-Th | H | |
| 2-514 | 6-Cl | 2-Th | H | |
| 2-515 | 7-Cl | 2-Th | H | |
| 2-516 | 8-Cl | 2-Th | H | |
| 2-517 | 5-F | 2-Th | H | |
| 2-518 | 6-F | 2-Th | H | |
| 2-519 | 7-F | 2-Th | H | |
| 2-520 | H | p-tolyl | H | |
| 2-521 | H | m-tolyl | H | |
| 2-522 | H | o-tolyl | H | |
| 2-523 | H | 4-tBu | H | |
| 2-524 | H | 4-C₆H₄Cl | H | |
| 2-525 | H | 3-C₆H₄Cl | H | |
| 2-526 | H | 2-C₆H₄Cl | H | |
| 2-527 | H | 4-C₆H₄F | H | |
| 2-528 | H | 3-C₆H₄F | H | |
| 2-529 | H | 2-C₆H₄F | H | |
| 2-530 | H | 4-C₆H₄OMe | H | |
| 2-531 | H | 3-C₆H₄OMe | H | |
| 2-532 | H | 2-C₆H₄OMe | H | |
| 2-533 | H | 4-C₆H₄CF₃ | H | |
| 2-534 | H | 3-C₆H₄CF₃ | H | |
| 2-535 | H | 2-C₆H₄CF₃ | H | |
| 2-536 | H | 2,3-C₆H₃Me₂ | H | |
| 2-537 | H | 2,4-C₆H₃Me₂ | H | |
| 2-538 | H | 2,5-C₆H₃Me₂ | H | |
| 2-539 | H | 2,6-C₆H₃Me₂ | H | |
| 2-540 | H | 3,4-C₆H₃Me₂ | H | |
| 2-541 | H | 3,5-C₆H₃Me₂ | H | |
| 2-542 | H | 2,3-C₆H₃Cl₂ | H | |
| 2-543 | H | 2,4-C₆H₃Cl₂ | H | |
| 2-544 | H | 2,5-C₆H₃Cl₂ | H | |
| 2-545 | H | 2,6-C₆H₃Cl₂ | H | |
| 2-546 | H | 3,4-C₆H₃Cl₂ | H | |
| 2-547 | H | 3,5-C₆H₃Cl₂ | H | |
| 2-548 | H | 2,4,6-C₆H₂Me₃ | H | |
| 2-549 | H | 3,4-C₆H₃(OMe)₂ | H | |
| 2-550 | 6,7-(CF₃)₂ | p-tolyl | H | |
| 2-551 | 5,7-(CF₃)₂ | m-tolyl | H | |
| 2-552 | 5,6-(CF₃)₂ | o-tolyl | H | |
| 2-553 | 7,8-(CF₃)₂ | 4-tBu | H | |
| 2-554 | 6,8-(CF₃)₂ | 4-C₆H₄Cl | H | |
| 2-555 | 5,8-(CF₃)₂ | 3-C₆H₄Cl | H | |

3. Biological Examples
3.1 Scoring of the Damage

The damage to the plants is assessed visually on a scale of 0-100% in comparison with control plants: 0%=no noticeable effect in comparison with the untreated plant 100%=the treated plant dies off.

3.2 Effect of the Herbicide and Effect of the Safener when Applied Post-Emergence

Seeds or rhizome pieces of mono- and dicotyledonous harmful plants and crop plants are placed in sandy loam soil in plastic pots, covered with soil and cultivated in a greenhouse under good growth conditions. Alternatively, harmful plants encountered in the cultivation of paddy rice are cultivated in pots where the water level is up to 2 cm above the soil surface. Three weeks after sowing, the test plants are treated at the three-leaf stage. The herbicide/safener active compound combinations according to the invention, formulated as emulsion concentrates, and, in parallel tests, the correspondingly formulated individual active compounds are sprayed onto the green parts of the plants in various dosages using an amount of water of 300 l/ha (converted) and, after the test plants have been kept in the greenhouse for 2-3 weeks under optimum growth conditions, the effect of the preparations is scored visually in comparison to untreated controls. In the case of rice or of harmful plants encountered in the cultivation of rice, the active compounds are also added directly to the irrigation water (application similar to granules application) or sprayed onto plants and into the irrigation water.

The tests show that safeners according to the invention, for example the compounds from table 1 of example numbers 5, 10, 15, 17, 29, 30, 45, 48, 65, 79, 81, 94, 97, 100, 114, 115, 121, 127, 128, 129, 131, 144, 151, 156, 158, 162, 163, 171, 172, 173, 174, 181, 191, 250, 273, 331, 334, 343, 351, 372, 374, 395, 405, 408, 412, 415, 416, 421, 431, 432, 434, 435, 436, 460, 465, 466, 478, 481, 482, 483, 486, 487, 501, 507, 513, 526, 530, 536, 539, 543, 546, 546a, 547, 548, 549, 560, 561, 562, 569, 570, 575a, 578, 579, 581a, 588, 595, 612, 638, 659, 687, 688, 689, 708, 813, 814, 815, 829, 835, 837, 847, 848, 855, 856, 857, 858, 861, 862, 876, 877, 883, 892, 893a, 904, 908, 909, 913, 921, 928, 932, 933, 943, 947, 948, 949, 969, 987, 988, 989, 999, 1000, 1001, 1010, 1011, 1014, 1027, 1028, 1033, 1034, 1035, 1036, 1039, 1040, 1041, 1042, 1049, 1050, 1051, 1052, 1053, 1054, 1055, 1061, 1063, 1073, 1075, 1081, 1082, 1083, 1084, 1087, 1088, 1102, 1103, 1104, 1105, 1106, 1108, 1110, 1111, 1112, 1113, 1114, 1115, 1119, 1120, 1126, 1129, 1130, 1131, 1134, 1137, 1138, 1139, 1140, 1141, 1145, 1146, 1148, 1150, 1151, 1152, 1153, 1165, 1175, 1179, 1196, 1197, 1199, 1207, 1208, 1209, 1210, 1218, 1221, 1229, 1233, 1234, 1238, 1243, 1244, 1245, 1250, 1251, 1252, 1259, 1261, 1262, 1268, 1269, 1272, 1275, 1276, 1277, 1278, 1279, 1280, 1283, 1284, 1287, 1288, 1289, 1294, 1295, 1296, 1297, 1298, 1299, 1300, 1301, 1303, 1304, 1305, 1306, 1307, 1308, 1309, 1310, 1311, 1312, 1314, 1315, 1316, 1317, 1318, 1319, 1320, 1321, 1322, 1323, 1324, 1325, 1327, 1328 1330, 1331, 1332, 1335, 1336, 1342, 1344, 1345, 1346, 1347, 1353, 1355, 1356, 1357, 1358, 1359, 1360, 1361, 1362, 1363, 1364, 1365, 1366, 1367, 1368, 1369, 1370, 1371, 1372, 1373, 1375, 1376, 1377, 1378, 1379, 1380, 1381, 1382, 1383, 1384, 1398, 1400, 1401, 1406, 1409, 1014, 1410, 1411, 1415, 1419, 2-1 and 2-2 in combination with herbicides, for example herbicides from the class of the HPPD inhibitors (for example compounds such as 2-{[5,8-dimethyl-1,1-dioxido-4-(pyrazin-2-yloxy)-3,4-dihydro-2H-thiochromen-6-yl]carbonyl}cyclohexane-1,3-dione from the class of the 2-aroylcyclohexanediones) in a ratio of herbicide:safener of 2:1 to 1:20 reduce herbicide damage to crop plants such as corn, rice, wheat or barley or other cereals considerably compared to the individual herbicides used without safener, i.e. the observed damage to the crop plant is reduced by 30 up to 100%. At the same time, the activity of the herbicide against economically important harmful plants is, if at all, not adversely affected to any significant extent, so that it is possible to achieve good herbicidal post-emergence action against a broad spectrum of weed grasses and broad-leaved weeds.

1. A method for protecting useful plants or crop plants against phytotoxic side effects of agrochemicals, which comprises applying an effective amount of a compound of the formula (I) or a salt thereof 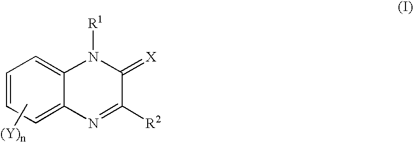
in which
X is oxygen or sulfur;
(Y)ₙ
are n substituents Y, where each Y independently of the others is a halogen, cyano, nitro, (C₁-C₆)-alkyl, (C₂-C₆)-alkenyl, (C₂-C₆)-alkynyl, (C₁-C₆)-alkoxy, (C₁-C₆)-alkylthio, (C₁-C₆)-alkylsulfinyl, (C₁-C₆)-alkylsulfonyl, (C₁-C₆)-alkoxycarbonyl, (C₁-C₄)-alkylamino or di-[(C₁-C₄)-alkyl]amino radical,
where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
(C₃-C₆)-cycloalkyl, (C₄-C₆)-cycloalkenyl, aryl or heterocyclyl,
where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkoxy-(C₁-C₄)-alkoxy and (C₁-C₄)-alkylthio, or
two adjacent groups Y together with the carbon atoms which are directly attached are a four- to eight-membered fused-on ring which is carbocyclic or heterocyclic, has one or more hetero ring atoms from the group consisting of N, O and S and is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio,
n is 0, 1, 2, 3 or 4,
R¹
is hydrogen, hydroxyl, amino, (C₁-C₄)-alkylamino, di-[(C₁-C₄)-alkyl]amino, (C₁-C₁₀)-alkyl, (C₃-C₁₀)-alkenyl, (C₃-C₁₀)-alkynyl or (C₁-C₁₀)-alkoxy,
where each of the 4 last-mentioned radicals is unsubstituted or substituted by one or more identical or different radicals R^{a} and, including substituents, has 1 to 30 carbon atoms, or
(C₃-C₁₀)-cycloalkyl, (C₄-C₁₀)-cycloalkenyl, aryl or heterocyclyl,
where each of the 4 last-mentioned radicals is unsubstituted or substituted by one or more identical or different radicals R^{b} and, including substituents, has 3 to 30 carbon atoms, and
R²
is hydrogen, (C₁-C₁₀)-alkyl, (C₃-C₁₀)-alkenyl or (C₃-C₁₀)-alkynyl,
where each of the 3 last-mentioned radicals is unsubstituted or substituted by one or more identical or different radicals R^{c} and, including substituents, has 1 to 30 carbon atoms, or
(C₃-C₁₀)-cycloalkyl, (C₄-C₁₀)-cycloalkenyl, aryl or heterocyclyl,
where each of the 4 last-mentioned radicals is unsubstituted or substituted by one or more identical or different radicals R^{d} and, including substituents, has 3 to 30 carbon atoms,
where in the radicals R¹ and R²
R^{a}, R^{b}, R^{c} and R^{d} are each an inorganic or organic radical,
as safener for preventing or reducing the phytotoxic actions of the agrochemicals on the useful plants or crop plants.

2. The method as claimed in claim 1, wherein
(Y)ₙ
are n substituents Y, where each Y independently of the others is a halogen, cyano, nitro, (C₁-C₄)-alkyl, (C₂-C₄)-alkenyl, (C₂-C₄)-alkynyl, (C₁-C₄)-alkoxy, (C₁-C₄)-alkylthio, (C₁-C₄)-alkylsulfinyl, (C₁-C₄)-alkylsulfonyl, (C₁-C₄)-alkoxycarbonyl, (C₁-C₄)-alkylamino or di-[(C₁-C₄)-alkyl]amino radical,
where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
(C₃-C₆)-cycloalkyl, (C₄-C₆)-cycloalkenyl, aryl or heterocyclyl,
where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio and, in the case of non-aromatic radicals, also oxo, or
two adjacent groups Y together with the carbon atoms which are directly attached are a four- to six-membered fused-on ring which is carbocyclic or heterocyclic, has one or more hetero ring atoms from the group consisting of N, O and S and is unsubstituted or substituted by one or more radicals from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio and oxo, and
n is 0, 1, 2, 3 or 4,
R¹
is hydrogen, hydroxyl, amino, (C₁-C₄)-alkylamino, di-[(C₁-C₄)-alkyl]amino, (C₁-C₆)-alkyl, (C₃-C₆)-alkenyl, (C₃-C₆)-alkynyl or (C₁-C₆)-alkoxy,
where each of the 4 last-mentioned radicals is unsubstituted or substituted by one or more identical or different radicals R^{a} and, including substituents, has 1 to 30 carbon atoms, or
(C₃-C₆)-cycloalkyl, (C₄-C₆)-cycloalkenyl, aryl or heterocyclyl,
where each of the 4 last-mentioned radicals is unsubstituted or substituted by one or more identical or different radicals R^{b} and, including substituents, has 3 to 30 carbon atoms, and
R²
is hydrogen, (C₁-C₁₀)-alkyl, (C₃-C₁₀)-alkenyl or (C₃-C₁₀)-alkynyl,
where each of the 3 last-mentioned radicals is unsubstituted or substituted by one or more identical or different radicals R^{c} and, including substituents, has 1 to 30 carbon atoms, or
(C₃-C₆)-cycloalkyl, (C₄-C₆)-cycloalkenyl, aryl or heterocyclyl,
where each of the 4 last-mentioned radicals is unsubstituted or substituted by one or more identical or different radicals R^{d} and, including substituents, has 3 to 30 carbon atoms,
where in the radicals R¹ and R² the substituent
R^{a} in each case independently of other radicals R^{a} is a radical from the group consisting of halogen, cyano, nitro and radicals of the formulae -Z^{a}-R^{a}* and R^{cyc-a},
R^{b} in each case independently of other radicals R^{b} is a radical from the group consisting of halogen, cyano, nitro and radicals of the formulae -Z^{b}-R^{b}* and R^{b}**,
R^{c} in each case independently of other radicals R^{c} is a radical from the group consisting of halogen, cyano, nitro and radicals of the formulae -Z^{c}-R^{c}* and R^{cyc-c},
R^{d} in each case independently of other radicals R^{d} is a radical from the group consisting of halogen, cyano, nitro and radicals of the formulae -Z^{d}-R^{d}* and R^{d}**
where in the radicals R^{a}, R^{b}, R^{c} and R^{d}
Z^{a}, Z^{b}, Z^{c} and Z^{d} in each case independently of one another are divalent groups of the formulae —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —S—CO—, —CO—S—, —S—CS—, —CS—S—, —O—CO—O—, —NR^{O}—, —O—NR^{O}—, —NR^{O}—O—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— and —SiR′R″—. where in each case p is the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)alkyl, (C₃-C₆)-cycloalkyl or acyl and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
R^{cyc-a} and R^{cyc-c} are an optionally substituted cyclic hydrocarbon radical having a total of 3 to 24 carbon atoms or an optionally substituted heterocyclic radical having a total of 1 to 24 carbon atoms and
R^{a}*, R^{b}*, R^{c}*, R^{d}*, R^{b}** and R^{c}** are each independently of one another an optionally substituted hydrocarbon radical having a total of 1 to 24 carbon atoms or an optionally substituted heterocyclic radical having a total of 1 to 24 carbon atoms or
R^{a}*, R^{b}*, R^{c}*, R^{d}* are each independently of one another hydrogen.

3. The method as claimed in claim 1, wherein
R¹
is hydrogen, (C₁-C₆)-alkyl, (C₃-C₆)-alkenyl or (C₃-C₆)-alkynyl,
where each of the 3 last-mentioned radicals is unsubstituted or substituted by one or more identical or different radicals R^{a} and, including substituents, has 1 to 24 carbon atoms, or
(C₃-C₆)-cycloalkyl or saturated heterocyclyl,
where each of the 2 last-mentioned radicals is unsubstituted or substituted by one or more identical or different radicals R and, including substituents, has 3 to 24 carbon atoms,
where
R^{a} is a radical from the group consisting of halogen, cyano, nitro, -Z^{a}-R^{a}* and R^{cyc-a} and
R^{b} is a radical from the group consisting of halogen, cyano, nitro, -Z^{b}-R^{b}* and R^{b}**,
where in the radicals R^{a} and R^{b}
Z^{a}, Z^{b} independently of one another are —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— and —SiR′R″—, where in each case p is the integer 0, 1 or 2 and the radicals R^{O} independently of one another are each hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl and R′ and R″ independently of one another are (C₁-C₄)-alkyl, phenyl, phenyl-(C₁-C₄)-alkyl or (C₃-C₆)-cycloalkyl,
R^{cyc-a} is (C₃-C₆)-cycloalkyl, (C₄-C₆)-cycloalkenyl, phenyl, saturated heterocyclyl, unsaturated non-aromatic heterocyclyl or heteroaryl, where each of the 6 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of saturated or unsaturated non-aromatic heterocyclyl, also oxo and
R²
is (C₁-C₁₀)-alkyl, (C₃-C₁₀)-alkenyl or (C₃-C₁₀)-alkynyl,
where each of the 3 last-mentioned radicals is unsubstituted or substituted by one or more identical or different radicals R^{c} and, including substituents, has 1 to 24 carbon atoms, or
(C₃-C₆)-cycloalkyl, aryl or heterocyclyl,
where each of the 3 last-mentioned radicals is unsubstituted or substituted by one or more identical or different radicals R^{d} and, including substituents, has 3 to 24 carbon atoms,
where
R^{c} in each case independently of the others is a radical from the group consisting of halogen, cyano, nitro and radicals of the formulae -Z^{c}-R^{c}* and R^{cyc-c},
R^{d} in each case independently of the others is a radical from the group consisting of halogen, cyano, nitro and radicals of the formulae -Z^{d}-R^{d}* and R^{d}**,
where in the radicals R^{c} and R^{d}
Z^{c} and Z^{d} each independently of one another are —O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —NR^{O}—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— and —SiR′R″—, where p is in each case the integer 0, 1 or 2 and the radicals R^{O} independently of one another are each hydrogen, (C₁-C₄)-alkyl, phenyl, phenyl-(C₁-C₄)-alkyl, (C₃-C₆)-cycloalkyl, (C₁-C₄)-alkanoyl, [(C₁-C₄)-alkoxy]carbonyl or (C₁-C₄)-alkylsulfonyl and R′ and R″ independently of one another are (C₁-C₄)-alkyl, phenyl, phenyl-(C₁-C₄)-alkyl or (C₃-C₆)-cycloalkyl,
R^{cyc-c} is (C₃-C₆)-cycloalkyl, (C₄-C₆)-cycloalkenyl, phenyl, saturated heterocyclyl, unsaturated non-aromatic heterocyclyl or heteroaryl, where each of the 6 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₁-C₄)-alkoxy-(C₁-C₄)-alkyl, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoyl and, in the case of saturated or unsaturated non-aromatic heterocyclyl, also oxo and
R^{c}*, R^{d}* and R^{d}** are each independently of one another (C₁-C₁₀)-alkyl, (C₃-C₁₀)-alkenyl, (C₃-C₁₀)-alkynyl, (C₃-C₆)-cycloalkyl, (C₄-C₆)-cycloalkenyl, aryl or heterocyclyl, where each of the 7 last-mentioned radicals is unsubstituted or substituted by one or more radicals from the group consisting of halogen, cyano, nitro, amino, hydroxyl, thio, (C₃-C₆)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylamino, di-[(C₁-C₄)-alkyl]amino, trimethylsilyl, (C₁-C₄)-alkanoyl, [(C₁-C₄)-alkoxy]carbonyl, di-[(C₁-C₄)-alkyl]carbamoylamino and, in the case of cyclic radicals, also (C₁-C₄)-alkyl, (C₁-C₄)-haloalkyl, (C₁-C₄)-alkoxy-(C₁-C₄)-alkyl and, in the case of heterocyclyl, also oxo or
R^{c}* and R^{d}* are each independently of one another hydrogen.

4. The method as claimed in claim 1, wherein the compound of the formula (I) or its salt is used in combination with a herbicide.

5. The method as claimed in claim 1, wherein the compound of the formula (I) or a salt thereof is used by the post-emergence method.

6. A compound of the formula (I) or a salt thereof 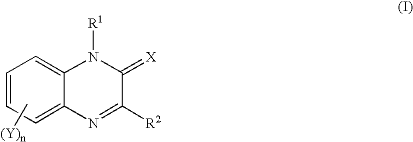
in which
X is oxygen or sulfur;
(Y)ₙ
are n substituents Y,
where each Y independently of the others is a halogen, cyano, nitro, (C₁-C₆)-alkyl, (C₂-C₆)-alkenyl, (C₂-C₆)-alkynyl, (C₁-C₆)-alkoxy, (C₁-C₆)-alkylthio, (C₁-C₆)-alkylsulfinyl, (C₁-C₆)-alkylsulfonyl, (C₁-C₆)-alkoxycarbonyl, (C₁-C₄)-alkylamino or di-[(C₁-C₄)-alkyl]amino radical,
where each of the 10 last-mentioned radicals is unsubstituted or substituted by one or more radicals from the group consisting of halogen, (C₁-C₄)-alkoxy, (C₁-C₄)-haloalkoxy and (C₁-C₄)-alkylthio, or
(C₃-C₆)-cycloalkyl, (C₄-C₆)-cycloalkenyl, aryl or heterocyclyl,
where each of the 4 last-mentioned radicals is unsubstituted or substituted by one or more radicals from the group consisting of halogen, cyano, nitro, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkoxy-(C₁-C₄)-alkoxy and (C₁-C₄)-alkylthio, or
two adjacent groups Y together with the carbon atoms which are directly attached are a four- to eight-membered fused-on ring which is carbocyclic or heterocyclic, has one to three hetero ring atoms from the group consisting of N, O and S and is unsubstituted or substituted by one or more radicals from the group consisting of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio,
n is 0, 1, 2, 3 or 4,
R¹
is (C₁-C₄)-alkyl, (C₃-C₁₀)-alkenyl or (C₃-C₁₀)-alkynyl,
where each of the two (2) last-mentioned radicals is unsubstituted or each of the three (3) last-mentioned radicals is substituted by one or more identical or different radicals R^{a} and, including substituents, has 1 to 30 carbon atoms, or
(C₃-C₁₀)-cycloalkyl, (C₄-C₁₀)-cycloalkenyl or saturated heterocyclyl,
where each of the 3 last-mentioned radicals is unsubstituted or substituted by one or more identical or different radicals R^{b} and, including substituents, has 3 to 30 carbon atoms, and
R²
is aryl or heterocyclyl,
where each of the 2 last-mentioned radicals is unsubstituted or substituted by one or more identical or different radicals R^{d} and, including substituents, has 3 to 30 carbon atoms,
where in the radicals R¹ and R² the substituent
R^{a} is in each case independently of the others a radical from the group consisting of halogen, cyano, nitro and radicals of the formulae -Z^{a}-R^{a}* and R^{cyc-a},
R^{b} is in each case independently of the others a radical from the group consisting of halogen, cyano, nitro and radicals of the formulae -Z^{b}-R^{b}* and R^{b}**,
R^{d} is in each case independently of the others a radical from the group consisting of halogen, cyano, nitro and radicals of the formulae -Z^{d}-R^{d}* and R^{d}**,
where in the radicals R^{a} and R^{b}
Z^{a} and Z^{b} are each independently of one another a divalent group of the formula
—O—, —S(O)ₚ—, —S(O)ₚ—O—, —O—S(O)ₚ—, —NR^{O}—S(O)ₚ—, —S(O)ₚNR^{O}—, —CO—, —O—CO—, —CO—O—, —S—CO—, —CO—S—, —S—CS—, —CS—S—, —O—CO—O—, —NR^{O}—, —O—NR^{O}—, —NR^{O}—O—, —NR^{O}—CO—, —CO—NR^{O}—, —O—CO—NR^{O}— or —NR^{O}—CO—O—, —NR^{O}—CO—NR^{O}—, —NR^{O}—CO—NR^{O}— and —SiR′R″—, where in each case p is the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms (and in this case preferably acyl from the group consisting of [(C₁-C₆)-alkyl]carbonyl, [(C₁-C₆)-alkoxy]carbonyl or [(C₁-C₆)-alkylsulfonyl) and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
R^{cyc-a} is an optionally substituted cyclic hydrocarbon radical having a total of 1 to 24 carbon atoms or an optionally substituted heterocyclic radical having a total of 1 to 24 carbon atoms and
R^{a}*, R^{b}* and R^{b}** are each independently of one another an optionally substituted hydrocarbon radical having a total of 1 to 24 carbon atoms or an optionally substituted heterocyclic radical having a total of 1 to 24 carbon atoms or
R^{a}* and R^{b}* are each independently of one another hydrogen, and
where in the radical R^{d}
Z^{d} is a divalent group of the formula —O—, —S(O)ₚ—, —S(O)ₚ—O—, —O—S(O)ₚ—, —S(O)ₚNR^{O}—, —CO—, —O—CO—, —CO—O—, —S—CO—, —CO—S—, —S—CS—, —CS—S—, —O—CO—O—, —CO—NR^{O}—, —O—CO—NR^{O}— or —SiR′R″— in which p is in each case the integer 0, 1 or 2 and the radicals R^{O} independently of one another are each hydrogen, (C₁-C₆)-alkyl, (C₂-C₆)-alkenyl, (C₂-C₆)-alkynyl, phenyl, phenyl-(C₁-C₆)-alkyl, (C₃-C₆)-cycloalkyl or acyl having preferably 1 to 10 carbon atoms and R′ and R″ independently of one another are (C₁-C₆)-alkyl, (C₂-C₆)-alkenyl, (C₂-C₆)-alkynyl, phenyl, phenyl-(C₁-C₆)-alkyl or (C₃-C₆)-cycloalkyl, and
R^{d}* and R^{d}** are each independently of one another an optionally substituted hydrocarbon radical having a total of 1 to 24 carbon atoms or an optionally substituted heterocyclic radical having a total of 1 to 24 carbon atoms or
R^{d}* is hydrogen,
except for compounds of the formula (I) and salts thereof in which
(a) R¹ is (C₁-C₄)-alkyl which is substituted by a cyclohexylcarbamoyl radical, and R² is a bicyclic heteroaryl radical,
(b) R¹ is (C₁-C₄)-alkyl which is substituted by a N-substituted carbamoyl radical and at the same time by optionally substituted cycloalkyl, heteroaryl or phenyl, and R² is phenyl,
(c) R¹ is (C₁-C₄)-alkyl which is substituted by 2-(trimethylsilyl)ethoxy, and R² is optionally substituted phenyl,
(d) R² is optionally substituted phenyl or heteroaryl, where one substituent contains more than one cyclic group or where two or more substituents are cyclic,
(e) R¹ is (C₁-C₄)-alkyl, which is substituted, and R² is phenyl which is substituted by iminocarbamoyl (amidine group),
(f) R¹ is (C₁-C₄)-alkyl which is substituted by an optionally substituted aryl radical, and R² is an optionally substituted aryl radical,
(g) R² is an optionally substituted indolyl radical or a N-(4-bromophenyl)- or N-phenyl-5-(hydroxymethyl)pyrazol-3-yl radical and
also except for the following compounds:
(h) 1-(2-hydroxyethyl)-3-phenylquinoxaline-2 (1H)-one,
(i) 1-[2-(diethylamino)ethyl]-3-phenylquinoxaline-2 (1H)-one,
(j) 1′-[3-(diethylamino)propyl]-3-phenylquinoxaline-2 (1H)-one,
(k) 7-chloro-1-[3-(dimethylamino)propyl]-3-phenylquinoxaline-2 (1H)-one,
(l) 1-{3-[2-(pyrrolidinyl-1-carbonyl)pyrrolidinyl-1-carbonyl]propyl}-3-phenylquinoxaline-2 (1H)-one,
(m) 1-{2-[2-(pyrrolidinyl-1-carbonyl)pyrrolidinyl-1-carbonyl]ethyl}-3-phenylquinoxaline-2 (1H)-one,
(n) 1-{2-[4-(pyrrolidinyl-1-carbonyl)thiazolidinyl-3-carbonyl]ethyl}-3-phenylquinoxaline-2 (1H)-one,
(o) 1-{2-[4-(thiazolidinyl-1-carbonyl)thiazolidinyl-3-carbonyl]ethyl}-3-phenylquinoxaline-2 (1H)-one,
(p) 1-{2-[4-(pyrrolidinyl-1-carbonyl)-1,1-dioxothiazolidinyl-3-carbonyl]ethyl}-3-phenylquinoxaline-2 (1H)-one,
(q) 1-[3-(amino)propyl]-3-phenylquinoxaline-2 (1H)-one,
(r) 1-(octahydro-2H-quinolizin-1-ylmethyl)-3-phenylquinoxaline-2 (1H)-one,
(s) 6-methoxy- or 6-methyl- or 6-trifluoromethyl- or 6-chloro-1-(octahydro-2H-quinolizin-1-ylmethyl)-3-phenylquinoxaline-2 (1H)-one (4 compounds),
(t) 1-(methylthiomethyl)-3-phenylquinoxaline-2 (1H)-one,
(u) 1-(methylaminocarbonylmethyl)-3-(2-ethoxyphenyl)quinoxaline-2 (1H)-one,
(v) 1-(dimethylaminomethyl)-3-(4-ethoxycarbonylphenyl)-6-bromoquinoxaline-2 (1H)-one,
(w) 1-(morpholin-4-ylmethyl)-3-(4-ethoxycarbonylphenyl)-6-bromoquinoxaline-2 (1H)-one,
(x) 1-(4-benzylpiperid-1-ylmethyl)-3-(4-ethylphenyl)quinoxaline-2 (1H)-one,
(y) 1-(4-benzylpiperazin-1-ylmethyl)-3-(3-chlorophenyl)quinoxaline-2 (1H)-one,
(z) 1-{3-[4-(4,5-dihydropyridazin-3 (2H)-on-6-yl)phenoxy]propyl}-3-phenylquinoxaline-2 (1H)-one.

7. A process for preparing a compound of the formula (I) or a salt thereof as defined in claim 6, which comprises
(a) reacting a compound of the formula (II) 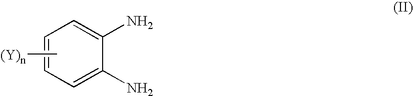
in which (Y)ₙ is as defined in formula (I)
with an α-keto acid derivative of the formula (III) 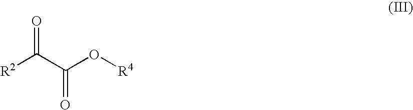
in which R² is as defined in formula (I) and R⁴ is hydrogen, optionally substituted alkyl or optionally substituted aryl
to give a compound of the formula (Ia) 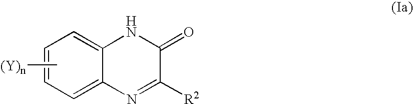
in which (Y)ₙ and R² are as defined in formula (I),
and converting this compound of the formula (Ia) by reaction with an alkylating agent of the formula (IV)
R¹-L (IV)
in which R¹ is as defined in formula (I) and L is a leaving group, or,
in the specific case where R¹ is a methyl group, using the alkylating agent dimethylformamide dimethyl acetal,
into the compound of the formula (I) or a salt thereof,
(b) reacting a compound of the formula (V) 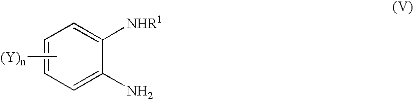
in which R¹ and (Y)ₙ are as defined in formula (I)
with an α-keto acid derivative of the formula (II) mentioned under (a) or
(c) derivatizing a compound of the formula (I′) 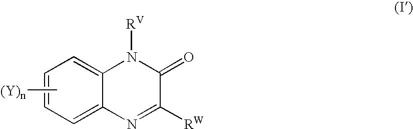
in which (Y)ₙ is as defined in formula (I),
the radical R^{v} is different from R¹ but a precursor of R¹ and the radical R^{w} is identical to R² or
the radical R^{w} is different from R² but a precursor of R² and the radical R^{v} is identical to R¹,
at the radical referred to as “precursor” by known or customary methods using one or more process steps, to give the compound of the formula (I).

8. A crop protection composition which comprises a compound of the formula (I) or a salt thereof as defined in claim 1 and a formulation auxiliary.

9. The crop protection composition as claimed in claim 8 which comprises a compound of the formula (I) or a salt thereof and one or more pesticides and, if appropriate, formulation auxiliaries.

10. A method as claimed in claim 1, which comprises applying an effective amount of one or more compounds of the formula (I) or salts thereof as defined in claim 1 before, after or simultaneously with the agrochemicals to the plants, parts of plants, plant seeds or seed.

11. The method as claimed in claim 10, wherein the application is by the post-emergence method.

12. The method as claimed in claim 10, wherein the application of the compound of the formula (I) is by treating the plant seeds or seed.

13. The method as claimed in claim 10, wherein the application is by the pre-emergence method.

14. A method for the selective control of harmful plants in crops of useful plants, which comprises applying an effective amount of one or more compounds of the formula (I) or salts thereof as defined in claim 1 before, after or simultaneously with one or more herbicides to the plants, parts of plants, plant seeds or seed.

15. The method as claimed in claim 14, wherein the seed is treated with one or more compounds of the formula (I) or salts thereof and the herbicide is applied after sowing by the pre-emergence method or by the post-emergence method.
